SEQUENCE LISTING (13 sequences)

SEQ ID NO: 1 - DNA, 426 bp, Psammomys obesus
  misc_feature (336)..(336): n = any nucleotide
ggacatcttttcagccatgaggagctttctggaaactcggagttgatacagaaatataggaatataatcacgcaggctcctaacctggagaacattgagctgtactggaacagctacaacaaccgccgagacctgaacttcgagcgaggtggtgagatgaccctcaagtgccctgtgatgctggtggtaggagaccaagcgcctcatgaggatgccgtggtggagtgtaactcaaaactggaccccacacagacctcgttcctcaagatggctgattctggaggtcagccacagctgacccagccaggcaagctgactgaggctttcaagtacttnctgcaaggcatgggctacatggcctcctcctgcatgactcgcctatcgaggtctcgcacggcatctttgaccagcgcagcatccattgat

SEQ ID NO: 2 - DNA, 451 bp, Psammomys obesus
gctggtaccggtccggaattcccgggatatcgtcgacccacgcgtccggtggtggagaagatcgctcctgccgtggttcacattgaactgtatcgcaaacttcctttctcgaagagggaggtgccagtggcgagtgggtccggatttatcgtgtctgaggatggactgattgtgaccaatgctcacgtggtgaccaacaaaaacagggtcaaggttgagctgaagaatggagcaacctatgaagctaaaatcaaggatgtggatgaaaaggcagacatcgcacttatcaaaattgaccaccagggaaagctgccagtcttgctgctgggccgctcctcagagcttcgaccaggagagtttgtggtcgccatcggaagccccttttcccttcaaaacacagtcaccactgggatcgtcagtaccacccagcgaggcggcaaagagctggggc

SEQ ID NO: 3 - DNA, 478 bp, Psammomys obesus
tgacattttctttccacctcttatgatagctgatatatactaaatctttatacagaaatgtcagtacttgaacaaattcaaaacacattggtttattaacttttggctcatgcatggtttattaggttcaaattatacctgattcatctatatttacttttaaaatgtgtggtttcctcattttaaaagtaaaactaaacagtgcttttggaatttctaagctactaattgttgatagatacagcctgtgtctagtaaaatagttttgtgggtgtgggttctatctttccatgaaaaagtgggaggtgtaagttagtttggttagtgcctaatagttaaatttatataaaataagaatgagcatttggtatctgtatgaaagggccctaaatcaaaatgattatccataatcaatctttattcttgttttataaaaaccaaagggcactcattggttaagtgtgctgagatagaaaag

SEQ ID NO: 4 - DNA, 1884 bp, Psammomys obesus
  misc_feature (609)..(609): n = any nucleotide
  misc_feature (611)..(611): n = any nucleotide
  misc_feature (1388)..(1388): n = any nucleotide
  misc_feature (1390)..(1390): n = any nucleotide
  misc_feature (1413)..(1413): n = any nucleotide
  misc_feature (1512)..(1512): n = any nucleotide
  misc_feature (1568)..(1568): n = any nucleotide
cgcagatactgcagagctgacgcattctttgcctggcatctcagcttgctaggtgtctcatcttcgctctgcgctcgtggcctcctccaaggcctccagtctcctttaagaaaactcaagacctgggaagctacgatgcgagcttgatgccgctaccctagcaggctatggacttcctgagggtcctcggactgttgacacccattccgatccgcatccttccaagctgataagcccgggaccctagggcggggtgcccagactcatgtgtgacgccttgcagtgaaaccccattcccagtgtggttgcttctttgctgggctttggcccatttgataccacgaaggatgacgatgctagttatgcagcagccaacacacctccccccaactctccgctgtcactggtgggccccactgtccaggaagcaggtgtccggaactgacatcttggagcagaggggccatgagaggtgtgtgtatcctgccagaaagcagctggaccacgacgctcccaagatgaacccactgtatacagaggcatcatgggagttgttatgtcaggagcattctagacccacgtgtacttgagcgtggaaagacagaaganangcgcagagactggggcacttgatctgctcaccatgatcgcctgcacgggtctcatccagttcctgccttaggctacagtggcggtgtccacgggcttgccattcaacgtgctctcagacccagatcgggctcaccactgaggagaaccttttcactttggtgggtatgcagagggaagggtctcgactccagagacctggagtccaaagtttgtttttttttatttaccagtaatttatttattttttttattattagttatgagtccaaagtttaataccattcaagctactgctgtctgtctgcttagagccacagcatgcaatgtggcaccaaggcatccttgtcctcacagtttcacactgtgggaacaggcatccttgttcttacagattagcgcgagggaaaccagaaatattaaacacgcagggttgtctctccaaagggagaggcacataccctgttttcctcccgaaggctgggagcggaggtgtttgatatcctggctacctctgtgcaatctgtaggccatgtccttaagatgtagctgtcagtcggtagtggagccggagccgtcagtcagtagattggggttgtggcatgcgcctttaactccatttaattccagcactctagtggtttggtacagcagcagcagcagcggttgcagtggcccggggaagtcctgaagaccagctttcatcccagcactcaggcagcaaaggcgggtggattctctgagttcgaggccggcctggtctacagagtgagtccagncnagccagggctacaacagagaaancctctctattgaaaaataaataaattataaaaaaaaaaaggtgtcatgtgtcctgtgtactttacaaagaatgttgatgcttaagcttttttgtgcacncaagaaaattgtttaactggtgtcagactcctgaagtttgaaccagcacttagccnggcgtggtggcgcacgcctgtaatcccagccctcgggaggcagaggcaggtggatctctgagttagaggccagcctggtctacagagtgagtccaggacagccaggattacacagagaaaccccgtctcaaaaatgtaaaataaattaaaataaagtttgaaccaacagtgtttactgagtcgtgttgaaacagattacctttttgcttctctttgatcattattctactgtggtgtcagcagagacccctccagcaggtggccaacgtgagagtctcaagcccgagaaggtaagaatttaaaaaaaaaaaaaaaa

SEQ ID NO: 5 - DNA, 1358 bp, Psammomys obesus
  misc_feature (385)..(385): n = any nucleotide
  misc_feature (416)..(416): n = any nucleotide
  misc_feature (430)..(430): n = any nucleotide
  misc_feature (507)..(507): n = any nucleotide
  misc_feature (754)..(754): n = any nucleotide
  misc_feature (856)..(856): n = any nucleotide
  misc_feature (928)..(928): n = any nucleotide
  misc_feature (980)..(980): n = any nucleotide
  misc_feature (1165)..(1165): n = any nucleotide
  misc_feature (1200)..(1200): n = any nucleotide
  misc_feature (1273)..(1273): n = any nucleotide
  misc_feature (1284)..(1284): n = any nucleotide
  misc_feature (1303)..(1303): n = any nucleotide
  misc_feature (1305)..(1305): n = any nucleotide
  misc_feature (1326)..(1326): n = any nucleotide
  misc_feature (1330)..(1330): n = any nucleotide
gttgggaaagaatgaagaaacaacccgatgaatagaaatgaaaagcctaagccaaatggatttctgttgagatgttggatgaaaacaagtatccactgtttaccaacttgacgaaaaatctcaactgaggtttggctgttaaaaaaaaaaattcactgtggcctctgtgcttaattgtcgtaaaccattgtgactgttactgctcaaagtatcgtactgttcattagtaactacatcagaattgcaccgctgctgttggaaaagccaataaagaaacccccagactgctgctcagcaaatgttaataaagtgtgcgcaccgtaggcctgtccacccagtcaccaagcagcgtccctttgtctgcgagtggctgtgggtgtgattnaccacctcagaggtgcacagcacctgcttgngcccttaagtgtgngtcagaagacaagcagcttctcggtaaccaacaacctgcttttcggagctcagtgtttaggctgtttactgaatcanatatgtaactcagcacacataagcgaagagagattttggctgcactggcaagagtgaaccaaatttacttctattttttaaaggcagatcatatttaagcatataagtaatttatggatataaattgttggatatttattttagtctgaatatttgtttttaaattattacatgtgttctctatgtctttatctctggaataacgatgccattaaccacatggccatatgttttgaaagttgggtgnaacagaggaaaagtcatccttcttggttcttgactccctttcctcaactacatgataagtctatcaataaagcatttgacctcagcaggggcagaagcctgnaaagttagaaaactcattgaccacagtagacaattgatttcttagaaataagaagtgagaagcagctgctgngctgagcaggggatgtaaaccaagtccagatgcaccaacgtgaagaggcttntagcaaaaatatgtttgcctctcacccctgcacatgttctagatgcttaaaaacagccacatggccccgctgcgaggacctcgtaatgttgttgttgttgttgttgttgttaaaggagttctcacaagcgtacaagtgcagcactgaaagtggctgaggcccacagtcctcagcacccaagtctnttccgcagcacgccaagctggtgttgtccgggtgngtatgtctgtgctcagtgccaagctggtgtttggtcccgttgtatattatgtgccccaagtgttttggggcanagctgacccangctggacacacttcttttngncttcgagtttactggttgatncagntaaaaataaattaattaattaaagactt

SEQ ID NO: 6 - DNA, 390 bp, Psammomys obesus
  misc_feature (3)..(3): n = any nucleotide
  misc_feature (9)..(9): n = any nucleotide
gtngaagcntaggagttcgaggatgcgcccgatgtcgagccgctggaacccacgcttagcaatatcatcgagcagcgcagccttaagtggatcttcgtcgggggcaagggtggcgttggtaagaccacctgcagctgcagcctggcggtccagctgtctaagggacgtgagagtgttctaatcatttccacagacccagctcacaacatctcagatgcatttgaccagaagttctccaaggtgcctaccaaggtcaaaggctatgacaacctctttgctatggagatagacccgagcctgggcgtggcagagctccctgatgaagttcttcgaggaagacaacatgctgagcatgggcaagaagatgatgcaggaggccatgagcgcctt

SEQ ID NO: 7 - DNA, 927 bp, Psammomys obesus
  misc_feature (524)..(524): n = any nucleotide
  misc_feature (527)..(527): n = any nucleotide
aaaattttacaaatgagtgtgaattgcattctgatataataattatcaccccaccacacttttactgacactgttgatggcctatgctgtgttttcacatcacaattcttgtatggaaaaatttctgtggcctgtgtaacccctctggtcagtattatgaaaccaactatctttggtgataaataaggttccggtaagatgcccagggttcatgagtatggcacaaataacagaggacaggaggccttcacgacgaaggagcccgtaagtggcctggagggcacagatgcagttccaggtcaagaaaagagcagctttttcaacaggcagtctgtgggtatgatgggaactcagcctgtctctgtagttatggacagcgtggcaggtgactgtgcccacatcttcctatacagtgctttttttttactgactggaagtacgtgaatctcacttagtccccaactggacgttttctggaaaaacaaagcaaatgttaaagtatgtctttctggatataggccagnagnaaatacattaagaatgagaggccttgctttgatctcagccattggaggctagaaaaaaattgaaaggaaccttcctgttgatagactcaaagccgtgaacagaagcctcttggcctgtttcagacaatctctggtaatctactgacaatatccaacagtttcgatgtccttgtttaactaccctggtagctttcttgtggatttgaagttcatttttaaagctgtggaatttcaaactgaattcacgtgcattttgtaaaagttcagaaccagtgctgagtctgtgtggcaggtttttttcaccgcgtgatatactattacaaatgcatgtggtgccatgcttgtcttcaaatatataagtagtgctaaatggataagtcatatggagcttttgatttag

SEQ ID NO: 8 - DNA, 374 bp, Psammomys obesus
  misc_feature (60)..(60): n = any nucleotide
tgagatagctactccataagcctctgaagagcaatagctaatttattattactgtaattnttttaaaggctttaaagtgcctcgggggttccttgaaactaattttctacttctgggattccctggattctttataagagatggtgacatgactagggaaattctttttttagtatgaaaattgtcccttcaatacttttctcttactggcattgaattatcacagagacagaaaattggtaatttttttaatttctaactctcccagaaaactcctcttgcctagtatttatttgatgtgctttaaccatgggaggaggggtggggggggaactcattcaagctgccagtattttgatctacaacctgtagca

SEQ ID NO: 9 - DNA, 402 bp, Psammomys obesus
  misc_feature (71)..(71): n = any nucleotide
  misc_feature (91)..(91): n = any nucleotide
  misc_feature (113)..(113): n = any nucleotide
  misc_feature (388)..(388): n = any nucleotide
cccacgcagtccgggtggctctgcagcacaatttaggccttggaggagctgtggttgtcaccctctacaanatgggcttccccgaagcggncagctccttcagaacacaccanatttcggctgctcccaccagctctgcaagggatggattcaaggccaatcttgtctttaaggagatcgagaagaagcttgaagaggaaggggaacagttcgtgaagaagatcggtgggatttttgccttcaaagtgaaggacggccctggaggcaaagaagccacctgggtggtggatgtgaagaatggcaagggatccgtgcttcccaactcagataagaaggctgactgcacaatcaccatggccgactccgacttgctggctctgatgactgncaaaatgaaccctc

SEQ ID NO: 10 - DNA, 18 bp, artificial sequence
  primer SP6
atttaggtgacactatag

SEQ ID NO: 11 - DNA, 20 bp, artificial sequence
  primer T7
taatacgactcactataggg

SEQ ID NO: 12 - protein, 4 aa, peptide
EVSC

SEQ ID NO: 13 - protein, 10 aa, peptide
GTRSRSHTSE

(19) United States
(12) Patent Application Publication
(10) Pub. No.: US 2007/0021589 A1
(43) Pub. Date: Jan. 25, 2007

(54) Obesity-related genes

(76) Inventors: Greg Collier, Ocean Grove (AU); Ken Walder, Ocean Grove (AU); David Segal, Ocean Grove (AU); Victoria C. Foletta, Ocean Grove (AU)

Correspondence Address: SCULLY, SCOTT, MURPHY & PRESSER, 400 GARDEN CITY PLAZA, SUITE 300, GARDEN CITY, NY, 11530, US

(21) Appl. No.: 10/541,998

(22) PCT Filed: Jan. 13, 2004
(86) PCT No.: PCT/AU04/00043

§ 371 Date: Jan. 17, 2006

(30) Foreign Application Priority Data
Jan. 13, 2003 (US) 60439767

(51) Int. Cl. C07K 14/705; A61K 38/17; C07H 21/04; C12P 21/06

(52) U.S. Cl. 530/350; 514/12; 435/69.1; 435/320.1; 435/325; 536/23.5

(57) ABSTRACT

The present invention relates generally to a nucleic acid molecule which is expressed in at least red gastrocnemius muscle or its equivalent under particular physiological conditions. It is proposed that the nucleic acid molecule is differentially expressed under differing conditions of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels. The subject nucleic acid molecule and/or its expression product is proposed to be used in therapeutic and diagnostic protocols for conditions such as healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels or as targets for the design and/or identification of modulators of their activity and/or function.

### BACKGROUND OF THE INVENTION

1. Field of the Invention

The present invention relates generally to a nucleic acid molecule which is expressed in at least red gastrocnemius muscle or its equivalent under particular physiological conditions. It is proposed that the nucleic acid molecule is differentially expressed under differing conditions of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels. The subject nucleic acid molecule and/or its expression product is proposed to be used in therapeutic and diagnostic protocols for conditions such as healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels or as targets for the design and/or identification of modulators of their activity and/or function.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The increasing sophistication of recombinant DNA technology is greatly facilitating research and development in the medical, veterinary and allied human and animal health fields. This is particularly the case in the investigation of the genetic bases involved in the etiology of certain disease conditions. One particularly significant condition from the stand point of morbidity and mortality is obesity and its association with type 2 diabetes (formerly non-insulin-dependent diabetes mellitus or NIDDM) and cardiovascular disease.

Obesity is defined as a pathological excess of body fat and is the result of an imbalance between energy intake and energy expenditure for a sustained period of time. Obesity is the most common metabolic disease found in affluent societies. The prevalence of obesity in these nations is alarmingly high, ranging from 10% to upwards of 50% in some sub-populations (Bouchard, The genetics of Obesity, Boca Raton: CRC Press, 1994). Of particular concern is the fact that the prevalence of obesity appears to be rising consistently in affluent societies and is now increasing rapidly in less prosperous nations as they become more affluent and/or adopt cultural practices similar to those in more affluent countries (Zimmet, Diabetes Care 15: 232-252, 1992). The escalating rates of obesity globally have resulted in the World Health Organisation declaring an obesity epidemic worldwide (World Trade Organisation. Obesity. Preventing and managing the global epidemic. Report of a WHO Consultation on Obesity. Geneva: World Health Organisation, 1998).

In Australia, the recent AusDiab study estimated that 7.5 million Australians (60%) aged 25 years and over were overweight or obese. Of these, 2.6 million (21%) were obese (BMI>30) (Dunstan et al., Diabetes Res. Clin. Pract. 57: 119-129, 2002). Similarly, the prevalence of obesity in the U.S. increased substantially between 1991 and 1998, increasing from 12% to 18% in Americans during this period (Mokdad et al, JAMA 282(16): 1519-1522, 1999).

The high and increasing prevalence of obesity has serious health implications for both individuals and society as a whole. Obesity is a complex and heterogeneous disorder and has been identified as a key risk indicator of preventable morbidity and mortality as obesity increases the risk of a number of other metabolic conditions including type 2 diabetes mellitus and cardiovascular disease (Must et al., JAMA 282(16): 1523-1529, 1999; Kopelman, Nature 404: 635-643, 2000). Alongside obesity the prevalence of diabetes continues to increase rapidly. The AusDiab survey estimated that close to 1 million Australians aged 25 years and over have type 2 diabetes (Dunstan et al., 2002). This represents approximately 7.5% of the population. In the U.S., the number of adults with diabetes increased by 49% between 1991 and 2000 (Marx, Science 686-689, 2002). It has been estimated that about 17 million people in the U.S. have type 2 diabetes and an equal number are thought to be pre-diabetic (Marx, 2002). In Australia, the annual costs of obesity associated with diabetes and other disease conditions has been conservatively estimated to be AUS$810 million for 1992-93 (National Health and Medical Research Council, Acting on Australia's weight: A strategy for the prevention of overweight and obesity. Canberra: National Health and Medical Research Council, 1996). The direct costs of diabetes and its complications in Australia in 1993-94 were estimated at $681 million, or 2.2% of total health system costs in that year (Australian Institute of Health and Welfare (AIWH), Australia's Health, 2002, Canberra: AIWH).

A genetic basis for the etiology of obesity is indicated inter alia from studies in twins, adoption studies and population-based analyses which suggest that genetic effects account for 25-80% of the variation in body weight in the general population (Bouchard, 1994, supra; Kopelman et al., Int. J. Obesity 18: 188-191, 1994; Ravussin, Metabolism 44(3): 12-14, 1995). It is considered that genes determine the possible range of body weight in an individual and then the environment influences the point within this range where the individual is located at any given time (Bouchard, 1994). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

A number of tissues have been implicated in the pathophysiology of obesity and type 2 diabetes, and of particular interest is the muscle. Skeletal muscle is the principle site of insulin-stimulated glucose disposal, accounting for approximately 75% of total glucose uptake. Skeletal muscle is also the major site of peripheral insulin resistance. Skeletal muscle also oxidizes free fatty acids for fuel, to meet its energy requirements. In healthy individuals, the muscle has the capacity to utilize both carbohydrate and lipids for energy and to fluctuate between these fuels in response to a range of signals including insulin concentrations. This metabolic flexibility is central to the role the muscle plays in whole body fuel metabolism and with diseases such as obesity and type 2 diabetes, this flexibility may be lost.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 3. A sequence listing is provided after the claims. A summary of genes identified in accordance with the present invention is provided in Table 1. Gene abbreviations are provided in Table 2.

In accordance with the present invention, genetic sequences were sought which are expressed in at least red gastrocnemius muscle of Psanimomys obesus (Israeli Sand Rat) under particular physiological conditions. Novel genes were then identified which have human and/or murine equivalents or homologs. In accordance with the present invention, genes are isolated which are proposed to be associated with one or more biological functions associated with disease conditions such as but not limited to healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

Analysis of genetic material from red gastrocnemius muscle tissue were used to identify candidate genetic sequences associated with a healthy state or with physiological conditions such as healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels. The Psammomys obesus animal model comprises three groups of animals designated Groups A, B and C based on metabolic phenotype as follows:

Group A: lean animals (normoglycernic; normoinsulinemic);

Group B: obese, non-diabetic animals (normoglycemic; hyperinsulinemic); and

Group C: obese, diabetic animals (hyperglycemic; hyperinsulinemic).

Microarray analysis was used to identify genetic sequences in fed and fasted mammals or in exercise trained and control mammals. Psammomys obesus was found to be particularly useful for this analysis.

cDNA microarray technology provides a powerful technical means to generate a gene expression database of both known genes and unknown transcripts. Using cDNA microarrays, comparative estimates can be obtained of the level of gene expression of large numbers of genes (up to 20,000 per microarray) in each sample. cDNA microarrays generally involve a large number of DNA “spots” in an orderly array chemically coupled to the surface of a solid substrate, usually but not exclusively an optically flat glass microscope slide. Fluorescently labeled cDNAs are generated from experimental and reference RNA samples and then competitively hybridized to the gene chip. The experimental and reference cDNAs are labeled with a different fluorescent dye and the intensity of each fluor at each DNA spot gives an indication of the level of that particular RNA species in the experimental sample relative to the reference RNA. The ratio of fluorescence can be taken as a measure of the expression level of the gene corresponding to that spot in the experimental sample.

In a preferred embodiment, nine expressed sequences were identified designated herein AGT-701 [SEQ ID NO:1], AGT-702 [SEQ ID NO:2], AGT-704 [SEQ ID NO:3], AGT-705 [SEQ ID NO:4], AGT-706 [SEQ ID NO:5], AGT-707 [SEQ ID NO:6], AGT-708 [SEQ ID NO:7], AGT-709 [SEQ ID NO:8] and AGT-710 [SEQ ID NO:9]. The corresponding expression products are provided in non-itallicized form, i.e. AGT-701, AGT-702, AGT-704, AGT-705, AGT-706, AGT-707, AGT-708, AGT-709, AGT-710.

A summary of the AGT genes is provided in Table 1.

The present invention contemplates the use of these sequences or their expression products in the manufacture of medicaments and diagnostic agents for a range of conditions including healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

The present invention provides, therefore, a nucleic acid molecule comprising a sequence of nucleotides encoding or complementary to a sequence encoding an expression product or a derivative, homolog, analog or mimetic thereof wherein said nucleic acid molecule or its homolog is differentially expressed in red gastrocnemius of P. obesus under fed or fasted or in exercise trained and control conditions.

The present invention further provides mammalian homology of the subject nucleic acid molecules such as human homology.

The present invention still further provides a nucleic acid molecule comprising a nucleotide sequence encoding or complementary to a sequence encoding an expression product or a derivative, homolog, analog or mimetic thereof wherein the nucleotide sequence is as substantially set forth in SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or a nucleotide sequence having at least about 30% identity to all or part of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or their complementary forms under low stringency conditions at 42° C. and wherein the nucleic acid molecule is differentially expressed in red gastocnemius tissue of P. obesus under fed or fasted or in exercise trained and control conditions.

The present invention also provides an isolated expression product or a derivative, homolog, analog or mimetic thereof which expression product is encoded by a nucleotide sequence which is differentially expressed in red gastrocnemius tissue of P. obesus under fed or fasted or in exercise trained and control conditions.

More particularly, the present invention is directed to an isolated expression product or a derivative, homolog, analog or mimetic thereof wherein the expression product is encoded by a nucleotide sequence substantially as set forth in SEQ ID NO:1, SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or a nucleotide sequence having at least 30% identity to all or part of SEQ ID NO:1, SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 and/or is capable of hybridizing to SEQ ID NO:1, SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or their complementary forms under low stringency conditions at 42° C.

The preferred genetic sequence of the present invention are referred to herein as AGT-701, AGT-702, AGT-704, AGT-705, AGT-706, AGT-707, AGT-708, AGT-709 and AGT-710. The expression products encoded by AGT-701, AGT-702, AGT-704, AGT-705, AGT-706, AGT-707, AGT-708, AGT-709 and AGT-710 are referred to herein as AGT-701, AGT-702, AGT-704, AGT-705, AGT-706, AGT-707, AGT-708, AGT-709 and AGT-710, respectively. The expression product may be an RNA (e.g. mRNA) or a protein. Where the expression product is an RNA, the present invention extends to RNA-related molecules associated thereto such as RNAi or intron or exon sequences therefrom.

Even yet another aspect of the present invention relates to a composition comprising AGT-701, AGT-702, AGT-704, AGT-705, AGT-706, AGT-707, AGT-708, AGT-709 and/or AGT-710 or its derivatives, homologs, analogs or mimetics or agonists or antagonists of AGT-701, AGT-702, AGT-704, AGT-705, AGT-706, AGT-707, AGT-708, AGT-709 and/or AGT-710 together with one or more pharmaceutically acceptable carriers and/or diluents.

The present invention is particularly directed to mammalian and in particular human homologs of the genes identified in P. obesus and their use or the use of expression products in therapy and diagnosis.

Another aspect of the present invention contemplates, therefore, a method for treating a subject comprising administering to said subject a treatment effective amount of AGT-701, AGT-702, AGT-704, AGT-705, AGT-706, AGT-707, AGT-708, AGT-709 and/or AGT-710 or a derivative, homolog, analog or mimetic thereof or a genetic sequence encoding same or an agonist or antagonist of AGT-701, AGT-702, AGT-704, AGT-705, AGT-706, AGT-707, AGT-708, AGT-709 and/or AGT-710 activity or AGT-701, AGT-702, AGT-704, AGT-705, AGT-706, AGT-707, AGT-708, AGT-709 and/or AGT-710 gene expression for a time and under conditions sufficient to effect treatment.

In an alternative embodiment, a molecule which modifies accumulation of one or more of AGT-701, AGT-702, AGT-704, AGT-705, AGT-706, AGT-707, AGT-708, AGT-709 and/or AGT-710. Such an agent may promote degradation of the target molecule, or may inhibit degradation.

In accordance with this and other aspects of the present invention, treatments contemplated herein include but are not limited to healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels. Treatment may be by the administration of a pharmaceutical composition or genetic sequences via gene therapy. Treatment is contemplated for human subjects as well as animals such as animals important to livestock industry.

A further aspect of the present invention is directed to a diagnostic agent for use in monitoring or diagnosing conditions such as but not limited to healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels, said diagnostic agent selected from an antibody to AGT-701, AGT-702, AGT-704, AGT-705, AGT-706, AGT-707, AGT-708, AGT-709 and/or AGT-710 or its derivatives, homologs, analogs or mimetics and a genetic sequence comprising or capable of annealing to a nucleotide strand associated with AGT-701, AGT-702, AGT-704, AGT-705, AGT-706, AGT-707, AGT-708, AGT-709 or AGT-710 useful inter alia in PCR, hybridization, RFLP analysis or AFLP analysis.

TABLE 1
Summary of AGT Genes

| GENE | SEQ ID NO: | HOMOLOG | DESCRIPTION |
|---|---|---|---|
| AGT-701 | 1 | human, mouse and rat | lower expression in Group C, fed |
| | | NDRG2 | animals and higher expression in |
| | | | Group B fasted animals; expression |
| | | | negatively correlated with body fat, |
| | | | body weight and blood glucose in |
| | | | fed animals; increases with exercise |
| | | | training |
| AGT-702 | 2 | human, mouse and rat | elevated expression after training; |
| | | PRSS11 | negative correlation with body |
| | | | weight and blood glucose; positive |
| | | | correlation with energy expenditure |
| AGT-704 | 3 | human PAI-RBP1 | decreased expression in Group C, |
| | | | fed animals; negative correlation |
| | | | with blood glucose in fed animals; |
| | | | increases with exercise training |
| AGT-705 | 4 | murine BC030414 | increased expression in Group C |
| | | | animals and in Group B fasted |
| | | | animals; expression negatively |
| | | | correlated with blood glucose; |
| | | | increases with exercise training |
| AGT-706 | 5 | human FL520069 | elevated expression in Group B |
| | | murine Ahi-1 | fasted and Group C fasted animals; |
| | | | expression negatively correlated |
| | | | with blood glucose in fed animals |
| | | | and positively correlated in insulin |
| | | | in fasted animals; increases with |
| | | | exercise training |
| AGT-707 | 6 | human ASNA1 | elevated expression in Group A |
| | | | animals; expression negatively |
| | | | correlated with body weight in fed |
| | | | animals; increases with exercise |
| | | | training |
| AGT-708 | 7 | human PKIA | elevated expression in Group B and |
| | | | Group C fasted animals; expression |
| | | | positively correlated with blood |
| | | | glucose |
| AGT-709 | 8 | human KIAA0633, | lower expression in Group C, fed |
| | | Mus musculus similar | animals; expression negatively |
| | | to KIAA0633 | correlated with body weight and |
| | | | blood glucose |
| AGT-710 | 9 | human, mouse and rat | lower expression in Group C, fed |
| | | SCP2 | animals; expression negatively |
| | | | correlated with body weight and |
| | | | blood glucose in fed animals; |
| | | | increases with exercise training |

TABLE 2
Gene Abbreviations

| ABBREVIATION | DEFINITION |
|---|---|
| NDRG2 | N-myc downstream-regulated gene 2 |
| PRSS11 | protease, serine 11 |
| PAI-RBP1 | PAI-1 mRNA binding protein |
| ASNA1 | human homolog of bacterial arsA arsenite |
| | transporter ATP binding |
| PKIA | protein kinase inhibitor alpha |
| SCP2 | sterol carrier protein 2 |

A summary of sequence identifiers used throughout the subject specification is provided in Table 3.

TABLE 3
Summary of Sequence Identifiers

| SEQUENCE | | |
|---|---|---|
| ID NO: | DESCRIPTION | SOURCE |
| 1 | Nucleotide sequence of AGT-701 | Psammomys obesus |
| 2 | Nucleotide sequence of AGT-702 | Psammomys obesus |
| 3 | Nucleotide sequence of AGT-704 | Psammomys obesus |
| 4 | Nucleotide sequence of AGT-705 | Psammomys obesus |
| 5 | Nucleotide sequence of AGT-706 | Psammomys obesus |
| 6 | Nucleotide sequence of AGT-707 | Psammomys obesus |
| 7 | Nucleotide sequence of AGT-708 | Psammomys obesus |
| 8 | Nucleotide sequence of AGT-709 | Psammomys obesus |
| 9 | Nucleotide sequence of AGT-710 | Psammomys obesus |
| 10 | primer SP6 | synthetic |
| 11 | primer T7 | synthetic |
| 12 | peptide | synthetic |
| 13 | peptide | synthetic |

### DETAILED DESCRIPTION OF THE PREFERRED EMBODIMENTS

The present invention is predicated in part on the identification of genes associated inter alia with regulation of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

Conveniently, an animal model may be employed to study the differences in gene expression in animal tissues such as red gastrocnemius under different conditions. In particular, the present invention is exemplified using the P. obesus (the Israeli Sand Rat) animal model of dietary-induced obesity and type 2 diabetes. In their natural desert habitat, an active lifestyle and saltbush diet ensure that they remain lean and normoglycemic (Shafrir and Gutman, J. Basic Clin. Physiol. Pharm. 4: 83-99, 1993). However, in a laboratory setting on a diet of ad libitum chow (on which many other animal species remain healthy), a range of pathophysiological responses are seen (Barnett et al., Diabetologia 37: 671-676, 1994a; Barnett et al., Int. J. Obesity 18: 789-794, 1994b; Barnett et al., Diabete Nutr. Metab. 8: 42-47, 1995). By the age of 16 weeks, more than half of the animals become obese and approximately one third develop type 2 diabetes. Only hyperphagic animals go on to develop hyperglycemia, highlighting the importance of excessive energy intake in the pathophysiology of obesity and type 2 diabetes in P. obesus (Collier et al., Ann. New York Acad. Sci. 827: 50-63, 1997a; Walder et al., Obesity Res. 5: 193-200, 1997a). Other phenotypes found include hyperinsulinemia, dyslipidemia and impaired glucose tolerance (Collier et al., 1997; Collier et al, Exp. Clin. Endocrinol. Diabetes 105: 36-37, 1997b). P. obesus exhibit a range of bodyweight and blood glucose and insulin levels which form a continuous curve that closely resembles the patterns found in human populations, including the inverted U-shaped relationship between blood glucose and insulin levels known as “Starling's curve of the pancreas” (Barnett et al., 1994a). It is the heterogeneity of the phenotypic response of P. obesus which makes it an ideal model to study the etiology and pathophysiology of obesity and type 2 diabetes.

The animals are conveniently classified into three groups designated Groups A, B and C:

Group A: animals are lean;

Group B: animals are obese and non-diabetic; and

Group C: animals are obese and diabetic.

In accordance with the present invention, a number of differentially expressed genetic sequences were identified in red gastrocnemius tissue in P. obesus under different feeding regimes (i.e. fed and fasted) or under exercise trained and control conditions. These genetic sequences have human and other animal homologs and, hence, the identification of these genetic sequences permits identification of genes involved in healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

Accordingly, one aspect of the present invention provides a nucleic acid molecule comprising a sequence of nucleotides encoding or complementary to a sequence encoding an expression product or a derivative, homolog, analog or mimetic thereof wherein said nucleic acid molecule is differentially expressed in red gastrocnemius muscle tissue of P. obesus under fed and fasted or in exercise trained and control conditions or a homolog of said nucleic acid molecule.

More particularly, the present invention provides a molecular marker for a physiological condition selected from a healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels, wherein said molecular marker comprises a nucleic acid molecule or an expression product of the nucleic acid molecule which are differentially expressed in at least liver, mesenteric adipose tissue and/or muscle.

The term “differentially expressed” is used in its most general sense and includes elevated levels of an expression product such as MRNA or protein or a secondary product such as cDNA in one tissue compared to another tissue or in the same tissue but under different conditions. Examples of different conditions includes differential expression in tissue from fed and fasted animals or in exercise trained and control animals. Differential expression is conveniently determined by a range of techniques including polymerase chain reaction (PCR) such as real-time PCR. Other techniques include suppression subtractive hyridization (SSH) and amplified fragment length polymorphism (AFLP) analysis. Microarray analysis of cDNA is particularly preferred.

A homolog refers to a genetic sequence in another animal or organism which has at least about 20% identity to the reference sequence. A preferred homolog is a human homolog.

It must be noted that, as used in the subject specification, the singular forms “a”, “an” and “the” include plural aspects unless the context clearly dictates otherwise. Thus, for example, reference to a “gene” includes a single gene, as well as two or more genes; reference to “an active agent” includes a single active agent, as well as two or more active agents; “a condition” includes a single condition or two or more conditions and so forth.

The expression product may be a protein or MRNA or may be an exon or intron spliced, for example, from an RNA construct. The expression product may also be a hairpin structure which includes or is associated with RNAi.

The selection of gastrocnemius is not intended to imply that different expression does not occur in other tissue.

The present invention further extends to homologs in other mammals and in particular humans as well as in other animals or organisms.

Another aspect of the present invention provides a nucleic acid molecule comprising a nucleotide sequence encoding or complementary to a sequence encoding an expression product or a derivative, homolog, analog or mimetic thereof wherein said nucleotide sequence is as substantially set forth in SEQ ID NO:1 (AGT-701) or SEQ ID NO:2 (AGT-702) or SEQ ID NO:3 (AGT-704) or SEQ ID NO:4 (AGT-705) or SEQ ID NO:5 (AGT-706) or SEQ ID NO:6 (AGT-707) or SEQ ID NO:7 (AGT-708) or SEQ ID NO:8 (AGT-709) or SEQ ID NO:9 (AGT-710) or a nucleotide sequence having at least about 30% identity to all or part of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 and/or is capable of hybridizing to one or more of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or their complementary forms under low stringency conditions at 42° C. and wherein said nucleic acid molecule is differentially expressed in red gastrocnemius muscle tissue under fed or fasted or in exercise trained and control conditions.

As indicated above, the preferred homology are derived from human, mouse, or rat, and more preferably human.

Reference herein to “similarity” is generally at a level of comparison of at least 15 consecutive or substantially consecutive nucleotides or at least 5 consecutive or substantially consecutive amino acid residues. Preferred percentage similarities have at least about 40%, at least about 50%, at least about 60%, at least about 70%, at least about 80% and at least about 90% or above. Examples include 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Reference herein to similarity is generally at a level of comparison of at least 15 consecutive or substantially consecutive nucleotides. It is particularly convenient, however, to determine similarity by comparing a total or complete sequence, after optimal alignment.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, Chapter 15, 1994-1998). A range of other algorithms may be used to compare the nucleotide and amino acid sequences such as but not limited to PILEUP, CLUSTALW, SEQUENCHER or VectorNTI.

The terms “sequence similarity” and “sequence identity” as used herein refers to the extent that sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C., such as 25, 26, 27, 28, 29, 30, 31, 32, 33, 34, 35, 36, 37, 38, 39, 40, 41 and 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, such as 16, 17, 18, 19, 20, 21, 22, 23, 24, 25, 26, 27, 28, 29 and 30% and from at least about 0.5 M to at least about 0.9 M salt, such as 0.5, 0.6, 0.7, 0.8 or 0.9 M for hybridization, and at least about 0.5 M to at least about 0.9 M salt, such as 0.5, 0.6, 0.7, 0.8 or 0.9 M for washing conditions, or high stringency, which includes and encompasses from at least about 31% v/v to at least about 50% v/v formamide, such as 31, 32, 33, 34, 35, 36, 37, 38, 39, 40, 41, 42, 43, 44, 45, 46, 47, 48, 49 and 50% and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3+0.41 (G+C)% (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974. Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

An expression product includes an RNA molecule such as an mRNA transcript as well as a protein. Some genes are non-protein encoding genes and produce mRNA or other RNA molecules and are involved in regulation by RNA:DNA, RNA:RNA or RNA:protein interaction. The RNA (e.g. mRNA) may act directly or via the induction of other molecules such as RNAi or via products mediated from splicing events (e.g. exons or introns). Other genes encode mRNA transcripts which are then translated into proteins. A protein includes a polypeptide. The differentially expressed nucleic acid molecules, therefore, may encode mRNAs only or, in addition, proteins. Both mRNAs and proteins are forms of “expression products”.

The nucleotide sequence or amino acid sequence of the present invention may correspond to exactly the same sequence of the naturally occurring gene (or corresponding cDNA) or protein or other expression product or may carry one or more nucleotide or amino acid substitutions, additions and/or deletions. The nucleotide sequences set forth in SEQ ID NO:1 (AGT-701), SEQ ID NO:2 (AGT-702) and SEQ ID NO:3 (AGT-704) or SEQ ID NO:4 (AGT-705) or SEQ ID NO:5 (AGT-706) or SEQ ID NO:6 (AGT-707) or SEQ ID NO:7 (AGT-708) or SEQ ID NO:8 (AGT-709) or SEQ ID NO:9 (AGT-710) correspond to novel genes referred to in parenthesis. The corresponding expression products are AGT-701, AGT-702, AGT-704, AGT-705, AGT-706, AGT-707, AGT-708, AGT-709 and AGT-710. Reference herein to AGT-701, AGT-702, AGT-704, AGT-705, AGT-706, AGT-707, AGT-708, AGT-709 and AGT-710 includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to AGT-701, AGT-702, AGT-704, AGT-705, AGT-706, AGT-707, AGT-708, AGT-709 and AGT-710.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:1 (AGT-701) or a derivative, homolog or mimetic thereof or having at least about 30% identity to all or part of SEQ ID NO:1 or a nucleotide sequence capable of hybridizing to SEQ ID NO:1 or its complementary form under low stringency conditions.

Yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:2 (AGT-702) or a derivative, homolog or mimetic thereof or having at least about 30% identity to all or part of SEQ ID NO:2 or a nucleotide sequence capable of hybridizing to SEQ ID NO:2 or its complementary form under low stringency conditions.

Still yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:3 (AGT-704) or a derivative, homolog or mimetic thereof or having at least about 30% identity to all or part of SEQ ID NO:3 or a nucleotide sequence capable of hybridizing to SEQ ID NO:3 or their complementary forms under low stringency conditions.

Even yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:4 (AGT-705) or a derivative, homolog or mimetic thereof or having at least about 30% identity to all or part of SEQ ID NO:4 or a nucleotide sequence capable of hybridizing to SEQ ID NO:4 or its complementary form under low stringency conditions.

Even still another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:5 (AGT-706) or a derivative, homolog or mimetic thereof or having at least about 30% identity to all or part of SEQ ID NO:5 or a nucleotide sequence capable of hybridizing to SEQ ID NO:5 or its complementary form under low stringency conditions.

Another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:6 (AGT-707) or a derivative, homolog or mimetic thereof or having at least about 30% identity to all or part of SEQ ID NO:6 or a nucleotide sequence capable of hybridizing to SEQ ID NO:6 or its complementary form under low stringency conditions.

A further aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:7 (AGT-708) or a derivative, homolog or mimetic thereof or having at least about 30% identity to all or part of SEQ ID NO:7 or a nucleotide sequence capable of hybridizing to SEQ ID NO:7 or its complementary form under low stringency conditions.

Yet another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:8 (AGT-709) or a derivative, homolog or mimetic thereof or having at least about 30% identity to all or part of SEQ ID NO:8 or a nucleotide sequence capable of hybridizing to SEQ ID NO:8 or its complementary form under low stringency conditions.

Still another aspect of the present invention provides a nucleic acid molecule or derivative, homolog or analog thereof comprising a nucleotide sequence encoding, or a nucleotide sequence complementary to a sequence encoding an expression product wherein said nucleotide sequence is substantially as set forth in SEQ ID NO:9 (AGT-710) or a derivative, homolog or mimetic thereof or having at least about 30% identity to all or part of SEQ ID NO:9 or a nucleotide sequence capable of hybridizing to SEQ ID NO:9 or its complementary form under low stringency conditions.

The expression pattern of AGT-701, AGT-702, AGT-704, AGT-705, AGT-706, AGT-707, AGT-708, AGT-709 and AGT-710 has been determined, inter alia, to indicate an involvement in the regulation of one or more of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels. In addition to the differential expression of AGT-701, AGT-702, AGT-704, AGT-705, AGT-706, AGT-707, AGT-708, AGT-709 and AGT-710 in red gastrocnemius muscle of fed versus fasted or exercise trained versus control animals, these genes may also be expressed in other tissues including but in no way limited to brain, muscle, adipose tissue, pancreas and gastrointestinal trait. The nucleic acid molecule corresponding to each of AGT-701, AGT-702, AGT-704, AGT-705, AGT-706, AGT-707, AGT-708, AGT-709 or AGT-710 is preferably a DNA such as a cDNA sequence or a genomic DNA. A genomic sequence may also comprise exons and introns. A genomic sequence may also include a promoter region or other regulatory regions.

A homolog is considered to be a gene from another animal species which has the same or greater than 30% similarity to one of AGT-701, AGT-702, AGT-704, AGT-705, AGT-706, AGT-707, AGT-708, AGT-709 and AGT-710 and/or which has a similar function. The above-mentioned genes are exemplified herein from P. obesus red gastrocnemius muscle. The present invention extends, however, to the homologous gene, as determined by nucleotide sequence and/or function, from humans, primates, livestock animals (e.g. cows, sheep, pigs, horses, donkeys), laboratory test animals (e.g. mice, guinea pigs, hamsters, rabbits), companion animals (e.g. cats, dogs) and captured wild animals (e.g. rodents, foxes, deer, kangaroos). Homologs may also be present in microorganisms and C. elegans.

The nucleic acids of the present invention and in particular AGT-701, AGT-702, AGT-704, AGT-705, AGT-706, AGT-707, AGT-708, AGT-709 and AGT-710 and their derivatives and homologs may be in isolated or purified form and/or may be ligated to a vector such as an expression vector. Expression may be in a eukaryotic cell line (e.g. mammalian, insect or yeast cells) or in prokaryote cells (e.g. E. coli) or in both. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure. The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector.

The derivatives of the nucleic acid molecule of the present invention include oligonucleotides, PCR primers, antisense molecules, molecules suitable for use in co-suppression and fusion nucleic acid molecules. Ribozymes and DNAzymes are also contemplated by the present invention directed to AGT-701, AGT-702, AGT-704, AGT-705, AGT-706, AGT-707, AGT-708, AGT-709 and AGT-710 or their mRNAs. Derivatives and homologs of AGT-701, AGT-702, AGT-704, AGT-705, AGT-706, AGT-707, AGT-708, AGT-709 and AGT-710 are conveniently encompassed by those nucleotide sequences capable of hybridizing to one or more of SEQ ID NO:1, SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or their complementary forms under low stringency conditions.

Derivatives include fragments, parts, portions, mutants, variants and mimetics from natural, synthetic or recombinant sources including fusion nucleic acid molecules. Derivatives may be derived from insertion, deletion or substitution of nucleotides.

Another aspect of the present invention provides an isolated expression product or a derivative, homolog, analog or mimetic thereof which is produced in larger or lesser amounts in red gastrocnemius muscle in obese animals compared to lean animals or in fed (including re-fed) compared to fasted animals or in animals under exercise trained compared to control conditions.

An expression product, as indicated above, may be RNA or protein. Insofar as the product is a protein, derivatives include amino acid insertional derivatives such as amino and/or carboxylic terminal fusions as well as intra-sequence insertions of single or multiple amino acids. Insertional amino acid sequence variants are those in which one or more amino acid residues are introduced into a predetermined site in a protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of protein forms of the expression products AGT-701, AGT-702, AGT-704, AGT-705, AGT-706, AGT-707, AGT-708, AGT-709 or AGT-710 should be understood as molecules exhibiting any one or more of the functional activities of these molecules and may be derived from any source such as being chemically synthesized or identified via screening processes such as natural product screening or screening of chemical libraries.

The derivatives include fragments having particular epitopes or parts of the entire protein fused to peptides, polypeptides or other proteinaceous or non-proteinaceous molecules.

Derivatives include fragments, parts, portions, mutants, polymorphisms, variants and mimetics from natural, synthetic or recombinant sources including fusion nucleic acid molecules. Derivatives may be derived from insertion, deletion or substitution of nucleotides.

Reference herein to AGT-701, AGT-702, AGT-704, AGT-705, AGT-706, AGT-707, AGT-708, AGT-709 or AGT-710 includes reference to isolated or purified naturally occurring AGT-701,AGT-702, AGT-704, AGT-705, AGT-706, AGT-707, AGT-708, AGT-709 or AGT-710 as well as any derivatives, homologs, analogs and mirnmetics thereof: Derivatives include parts, fragments and portions AGT-701, AGT-702, AGT-704, AGT-705, AGT-706, AGT-707, AGT-708, AGT-709 or AGT-710 as well as single and multiple amino acid substitutions, deletions and/or additions to AGT-701, AGT-702, AGT-704, AGT-705, AGT-706, AGT-707, AGT-708, AGT-709 and AGT-710 when the expression products are proteins. A derivative of AGT-701, AGT-702, AGT-704, AGT-705, AGT-706, AGT-707, AGT-708, AGT-709 or AGT-710 is conveniently encompassed by molecules encoded by a nucleotide sequence capable of hybridizing to SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 under low stringency conditions.

Other derivatives of AGT-701, AGT-702, AGT-704, AGT-705, AGT-706, AGT-707, AGT-708, AGT-709 and AGT-710 include chemical analogs. Analogs of AGT-701, AGT-702, AGT-704, AGT-705, AGT-706, AGT-707, AGT-708, AGT-709 and AGT-710 contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulfonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 4.

TABLE 4
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

All such modifications may also be useful in stabilizing the AGT-701, AGT-702, AGT-704, AGT-705, AGT-706, AGT-707, AGT-708, AGT-709 and AGT-710 molecule for use in in vivo administration protocols or for diagnostic purposes.

As stated above, the expression product may be a RNA or protein.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the expression product is encoded by a sequence of nucleotides comprising SEQ ID NO:1 or a derivative, homolog or analog thereof including a nucleotide sequence having at least about 30% identity to SEQ ID NO:1 or a nucleotide sequence capable of hybridizing to SEQ ID NO:1 or its complementary form under low stringency conditions.

In another particularly preferred embodiment, the expression product is encoded by a sequence of nucleotides comprising SEQ ID NO:2 or a derivative, homolog or analog thereof including a nucleotide sequence having at least about 30% identity to SEQ ID NO:2 or a nucleotide sequence capable of hybridizing to SEQ ID NO:2 or its complementary form under low stringency conditions.

In still another particularly preferred embodiment, the expression product is encoded by a sequence of nucleotides comprising SEQ ID NO:3 or a derivative homolog or analog thereof including a nucleotide sequence having at least about 30% identity to SEQ ID NO:3 or a nucleotide sequence capable of hybridizing to SEQ ID NO:3 or their complementary form under low stringency conditions.

In yet another particularly preferred embodiment, the expression product is encoded by a sequence of nucleotides comprising SEQ ID NO:4 or a derivative homolog or analog thereof including a nucleotide sequence having at least about 30% identity to SEQ ID NO:4 or a nucleotide sequence capable of hybridizing to SEQ ID NO:4 or their complementary form under low stringency conditions.

In another particularly preferred embodiment, the expression product is encoded by a sequence of nucleotides comprising SEQ ID NO:5 or a derivative homolog or analog thereof including a nucleotide sequence having at least about 30% identity to SEQ ID NO:5 or a nucleotide sequence capable of hybridizing to SEQ ID NO:5 or its complementary form under low stringency conditions.

In still another particularly preferred embodiment, the expression product is encoded by a sequence of nucleotides comprising SEQ ID NO:6 or a derivative homolog or analog thereof including a nucleotide sequence having at least about 30% identity to SEQ ID NO:6 or a nucleotide sequence capable of hybridizing to SEQ ID NO:6 or its complementary form under low stringency conditions.

In a further particularly preferred embodiment, the expression product is encoded by a sequence of nucleotides comprising SEQ ID NO:7 or a derivative homolog or analog thereof including a nucleotide sequence having at least about 30% identity to SEQ ID NO:7 or a nucleotide sequence capable of hybridizing to SEQ ID NO:7 or its complementary form under low stringency conditions.

In still yet another particularly preferred embodiment, the expression product is encoded by a sequence of nucleotides comprising SEQ ID NO:8 or a derivative homolog or analog thereof including a nucleotide sequence having at least about 30% identity to SEQ ID NO:8 or a nucleotide sequence capable of hybridizing to SEQ ID NO:8 or its complementary form under low stringency conditions.

In yet another particularly preferred embodiment, the expression product is encoded by a sequence of nucleotides comprising SEQ ID NO:9 or a derivative homolog or analog thereof including a nucleotide sequence having at least about 30% identity to SEQ ID NO:9 or a nucleotide sequence capable of hybridizing to SEQ ID NO:9 or its complementary form under low stringency conditions.

Higher similarities are also contemplated by the present invention such as greater than 40% or 50% or 60% or 70% or 80% or 90% or 95% or 96% or 97% or 98% or 99% or above. Further examples include 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

Another aspect of the present invention is directed to an isolated expression product selected from the list consisting of:
(i) an MRNA or protein encoded by a novel nucleic acid molecule which molecule is differentially expressed in red gastrocnemius muscle from P. obesus animals under fed or fasting conditions or in exercise trained and control animals or a derivative, homolog, analog, chemical equivalent or mimetic thereof;
(ii) an MRNA or protein encoded by a novel nucleic acid molecule which molecule is differentially expressed in red gastrocnemius muscle from P. obesus animals under fed or fasting conditions or in exercise trained and control animals or a derivative, homolog, analog, chemical equivalent or mimetic thereof;
(iii) AGT-701, AGT-702, AGT-704, AGT-705, AGT-706, AGT-707, AGT-708, AGT-709 or AGT-710 or a derivative, homolog, analog, chenical equivalent or mimetic thereof;
(iv) a protein encoded by a nucleotide sequence comprising SEQ ID NO:1 or a derivative, homolog or analog thereof or a sequence encoding an amino acid sequence having at least about 30% similarity to this sequence or a derivative, homolog, analog, chemical equivalent or mimetic of said protein;
(vi) a protein encoded by a nucleotide sequence substantially comprising SEQ ID NO:2 or a derivative, homolog or analog thereof or a sequence encoding an amino acid sequence having at least about 30% similarity to this sequence or a derivative, homolog, analog, chemical equivalent or mimetic of said protein;
(vii) a protein encoded by a nucleotide sequence substantially comprising SEQ ID NO:3 or a derivative, homolog or analog thereof or a sequence encoding an amino acid sequence having at least about 30% similarity to these sequences or a derivative, homolog, analog, chemical equivalent or mimetic of said protein;
(viii) a protein comprising an amino acid sequence substantially as set forth in SEQ ID NO:4 or a derivative, homolog or analog thereof or a sequence encoding an amino acid sequence having at least about 30% similarity to these sequences or a derivative, homolog, analog, chemical equivalent or mimetic of said protein;
(ix) a protein encoded by a nucleotide sequence substantially comprising SEQ ID NO:5 or a derivative, homolog or analog thereof or a sequence encoding an amino acid sequence having at least about 30% similarity to this sequence or a derivative, homolog, analog, chemical equivalent or mimetic of said protein;
(x) a protein encoded by a nucleotide sequence substantially comprising SEQ ID NO:6 or a derivative, homolog or analog thereof or a sequence encoding an amino acid sequence having at least about 30% similarity to this sequence or a derivative, homolog, analog, chemical equivalent or mimetic of said protein;
(xi) a protein encoded by a nucleotide sequence substantially comprising SEQ ID NO:7 or a derivative, homolog or analog thereof or a sequence encoding an amino acid sequence having at least about 30% similarity to this sequence or a derivative, homolog, analog, chemical equivalent or mimetic of said protein;
(xii) a protein encoded by a nucleotide sequence substantially comprising SEQ ID NO:8 or a derivative, homolog or analog thereof or a sequence encoding an amino acid sequence having at least about 30% similarity to this sequence or a derivative, homolog, analog, chemical equivalent or mimetic of said protein;
(xiii) a protein encoded by a nucleotide sequence substantially comprising SEQ ID NO:9 or a derivative, homolog or analog thereof or a sequence encoding an amino acid sequence having at least about 30% similarity to this sequence or a derivative, homolog, analog, chemical equivalent or mimetic of said protein;
(xiv) a protein encoded by a nucleic acid molecule capable of hybridizing to a nucleotide sequence comprising SEQ ID NO:1 or its complementary form or a derivative, homolog or analog thereof under low stringency conditions;
(xv) a protein encoded by a nucleic acid molecule capable of hybridizing to a nucleotide sequence comprising SEQ ID NO:2 or its complementary form or a derivative, homolog or analog thereof under low stringency conditions;
(xvi) a protein encoded by a nucleic acid molecule capable of hybridizing to a nucleotide sequence comprising SEQ ID NO:3 or their complementary forms or a derivative, homolog or analog thereof under low stringency conditions;
(xvii) protein encoded by a nucleic acid molecule capable of hybridizing to a nucleotide sequence comprising SEQ ID NO:4 or their complementary forms or a derivative, homolog or analog thereof under low stringency conditions;
(xviii) a protein encoded by a nucleic acid molecule capable of hybridizing to a nucleotide sequence comprising SEQ ID NO:5 or its complementary form or a derivative, homolog or analog thereof under low stringency conditions;
(xix) a protein encoded by a nucleic acid molecule capable of hybridizing to a nucleotide sequence comprising SEQ ID NO:6 or its complementary form or a derivative, homolog or analog thereof under low stringency conditions;
(xx) a protein encoded by a nucleic acid molecule capable of hybridizing to a nucleotide sequence comprising SEQ ID NO:7 or its complementary form or a derivative, homolog or analog thereof under low stringency conditions;
(xxi) a protein encoded by a nucleic acid molecule capable of hybridizing to a nucleotide sequence comprising SEQ ID NO:8 or its complementary form or a derivative, homolog or analog thereof under low stringency conditions; and
(xxii) a protein encoded by a nucleic acid molecule capable of hybridizing to a nucleotide sequence comprising SEQ ID NO:9 or its complementary form or a derivative, homolog or analog thereof under low stringency conditions.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% of subject protein relative to other components as determined by molecular weight, amino acid sequence or other convenient means. The protein of the present invention may also be considered, in a preferred embodiment, to be biologically pure.

Without limiting the theory or mode of action of the present invention, the expression of AGT-701, AGT-702, AGT-704, AGT-705, AGT-706, AGT-707, AGT-708, AGT-709 and AGT-710 is thought to relate to regulation of body weight and glucose homeostasis. Modulation of expression of these genes is thought inter alia to regulate energy balance via effects on energy intake and also effects on carbohydrate/fat metabolism. The energy intake effects are likely to be mediated via the central nervous system but peripheral effects on the metabolism of both carbohydrate and fat are possible. The expression of these genes may also be regulated by fasting and feeding. Accordingly, regulating the expression and/or activity of these genes or their expression products provides a mechanism for regulating both body weight and energy metabolism, including carbohydrate and fat metabolism.

The identification of AGT-701, AGT-702, AGT-704, AGT-705, AGT-706, AGT-707, AGT-708, AGT-709 and AGT-710 permits the generation of a range of therapeutic molecules capable of modulating expression of AGT-701, AGT-702, AGT-704, AGT-705, AGT-706, AGT-707, AGT-708, AGT-709 and AGT-710 or modulating the activity of AGT-701, AGT-702, AGT-704, AGT-705, AGT-706, AGT-707, AGT-708, AGT-709 and AGT-710 and/or which modulate levels of the expression products (i.e. agents which affect the accumulation of the products). Modulators contemplated by the present invention include agonists and antagonists of AGT-701, AGT-702, AGT-704, AGT-705, AGT-706, AGT-707, AGT-708, AGT-709 and AGT-710 expression. Antagonists of AGT-701, AGT-702, AGT-704, AGT-705, AGT-706, AGT-707, AGT-708, AGT-709 and AGT-710 expression include antisense molecules, ribozymes and co-suppression molecules (including any molecules which induce RNAi). Agonists include molecules which increase promoter activity or which interfere with negative regulatory mechanisms. Antagonists of AGT-701, AGT-702, AGT-704, AGT-705, AGT-706, AGT-707, AGT-708, AGT-709 and AGT-710 include antibodies and inhibitor peptide fragments. All such molecules may first need to be modified to enable such molecules to penetrate cell membranes. Alternatively, viral agents may be employed to introduce genetic elements to modulate expression of AGT-701, AGT-702, AGT-704, AGT-705, AGT-706, AGT-707, AGT-708, AGT-709 and AGT-710. In so far as AGT-701, AGT-702, AGT-704, AGT-705, AGT-706, AGT-707, AGT-708, AGT-709 and AGT-710 act in association with other genes such as the ob gene which encodes leptin, the therapeutic molecules may target AGT-701, AGT-702, AGT-704, AGT-705, AGT-706, AGT-707, AGT-708, AGT-709 and AGT-710 and ob genes or their translation products.

The present invention contemplates, therefore, a method for modulating expression of AGT-701, AGT-702, AGT-704, AGT-705, AGT-706, AGT-707, AGT-708, AGT-709 and AGT-710 in a mammal, said method comprising contacting the AGT-701, AGT-702, AGT-704, AGT-705, AGT-706, AGT-707, AGT-708, AGT-709 and AGT-710 gene with an effective amount of a modulator of AGT-701, AGT-702, AGT-704, AGT-705, AGT-706, AGT-707, AGT-708, AGT-709 and AGT-710 expression for a time and under conditions sufficient to up-regulate or down-regulate or otherwise modulate expression of AGT-701, AGT-702, AGT-704, AGT-705, AGT-706, AGT-707, AGT-708, AGT-709 and AGT-710.

For example, a nucleic acid molecule encoding AGT-701, AGT-702, AGT-704, AGT-705, AGT-706, AGT-707, AGT-708, AGT-709 and AGT-710 or a derivative or homolog thereof may be introduced into a cell to enhance the ability of that cell to produce AGT-701, AGT-702, AGT-704, AGT-705, AGT-706, AGT-707, AGT-708, AGT-709 and AGT-710, conversely, AGT-701, AGT-702, AGT-704, AGT-705, AGT-706, AGT-707, AGT-708, AGT-709 and AGT-710 sense and/or antisense sequences such as oligonucleotides may be introduced to decrease expression of the genes at the level of transcription, post-transcription or translation. Sense sequences preferably encode hair pin RNA molecules or double-stranded RNA molecules.

Another aspect of the present invention contemplates a method of modulating activity of AGT-701, AGT-702, AGT-704, AGT-705, AGT-706, AGT-707, AGT-708, AGT-709 and AGT-710 in a mammal, said method comprising administering to said mammal a modulating effective amount of a molecule for a time and under conditions sufficient to increase or decrease AGT-701, AGT-702, AGT-704, AGT-705, AGT-706, AGT-707, AGT-708, AGT-709 and AGT-710 activity. The molecule may be a proteinaceous molecule or a chemical entity and may also be a derivative of AGT-701, AGT-702, AGT-704, AGT-705, AGT-706, AGT-707, AGT-708, AGT-709 and AGT-710 or its ligand.

Still another aspect of the present invention contemplates a method of modulating the accumulation of AGT-701, AGT-702, AGT-704, AGT-705, AGT-706, AGT-707, AGT-708, AGT-709 and AGT-710 in a mammal, said method comprising administering to said mammal a modulating effective amount of a molecule for a time and under conditions sufficient to increase or decrease AGT-701, AGT-702, AGT-704, AGT-705, AGT-706, AGT-707, AGT-708, AGT-709 and AGT-710 levels.

Modulating levels of AGT-701, AGT-702, AGT-704, AGT-705, AGT-706, AGT-707, AGT-708, AGT-709 and AGT-710 expression or AGT-701, AGT-702, AGT-704, AGT-705, AGT-706, AGT-707, AGT-708, AGT-709 and/or AGT-710 activity or function is important in the treatment of a range of conditions such as healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels. It may also be useful in the agricultural industry to assist in the generation of leaner animals, or where required, more obese animals. Accordingly, mammals contemplated by the present invention include but are not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates therapeutic and prophylactic use of AGT-701, AGT-702, AGT-704, AGT-705, AGT-706, AGT-707, AGT-708, AGT-709 and/or AGT-710 expression products or AGT-701, AGT-702, AGT-704, AGT-705, AGT-706, AGT-707, AGT-708, AGT-709 and AGT-710 genetic mutants and/or agonists or antagonists agents thereof.

The present invention contemplates, therefore, a method of modulating expression of AGT-701, AGT-702, AGT-704, AGT-705, AGT-706, AGT-707, AGT-708, AGT-709 and/or AGT-710 in a mammal, said method comprising contacting the AGT-701, AGT-702, AGT-704, AGT-705, AGT-706, AGT-707, AGT-708, AGT-709 and/or AGT-710 genes with an effective amount of an agent for a time and under conditions sufficient to up-regulate, down-regulate or otherwise module expression of AGT-701, AGT-702, AGT-704, AGT-705, AGT-706, AGT-707, AGT-708, AGT-709 and AGT-710.

Another aspect of the present invention contemplates a method of modulating activity of AGT-701, AGT-702, AGT-704, AGT-705, AGT-706, AGT-707, AGT-708, AGT-709 and/or AGT-710 in a subject, said method comprising administering to said subject a modulating effective amount of an agent for a time and under conditions sufficient to increase or decrease AGT-701, AGT-702, AGT-704, AGT-705, AGT-706, AGT-707, AGT-708, AGT-709 and/or AGT-710 activity or function.

Modulation of activity by the administration of an agent to a mammal can be achieved by one of several techniques, including, but in no way limited to, introducing into a mammal a proteinaceous or non-proteinaceous molecule which:
(i) modulates expression of AGT-701, AGT-702, AGT-704, AGT-705, AGT-706, AGT-707, AGT-708, AGT-709 and/or AGT-710;
(ii) functions as an antagonist of AGT-701, AGT-702, AGT-704, AGT-705, AGT-706, AGT-707, AGT-708, AGT-709 and/or AGT-710; and/or
(iii) functions as an agonist of AGT-701, AGT-702, AGT-704, AGT-705, AGT-706, AGT-707, AGT-708, AGT-709 and/or AGT-710.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful in a therapeutic or prophylactic treatment of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

Accordingly, another aspect of the present invention relates to a method of treating a mammal suffering from a condition characterized by one or more symptoms of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels, said method comprising administering to said mammal an effective amount of an agent for a time and under conditions sufficient to modulate the expression of AGT-701, AGT-762, AGT-704, AGT-705, AGT-706, AGT-707, AGT-708, AGT-709 and/or AGT-710 or sufficient to modulate the activity of AGT-701, AGT-702, AGT-704, AGT-705, AGT-706, AGT-707, AGT-708, AGT-709 and/or AGT-710.

In another aspect, the present invention relates to a method of treating a mammal suffering from a disease condition characterized by one or more symptoms of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels, said method comprising administering to said mammal an effective amount of AGT-701, AGT-702, AGT-704, AGT-705, AGT-706, AGT-707, AGT-708, AGT-709 and/or AGT-710 or AGT-701, AGT-702 AGT-704, AGT-705, AGT-706, AGT-707, AGT-708, AGT-709 and/or AGT-710.

As used herein “myopathy” refers to any abnormal conditions or disease of the muscle tissues, which include the muscles over our bones (skeletal muscle) and the heart (cardiac muscle).

Obesity, inter alia myopathy, anorexia, diabetes and disorders associated with imbalances in metabolic energy levels, including any condition associated with varying levels of selenoproteins are disease and disorders associated with mitochondrial dysfunction, and genetic disorders. Mitochondria are part of the cell (organelle) that is responsible for energy production. The organelle consists of two sets of membranes, a smooth continuous outer coat and an inner membrane arranged in tubules or in folds that form plate-like double membranes (cristae). Mitochondria are the principal energy source of the cell, containing the cytochrome enzymes of terminal electron transport and the enzymes of the citric acid cycle, fatty acid oxidation, and oxidative phosphorylation. They are responsible for converting nutrients into energy as well as many other specialized tasks. Mitochondria are complex organelles located in virtually all cells of the body. A large degree of their complexity is due to the fact that over 1000 proteins are located in the mitochondria. Thirteen of these proteins are encoded by the mitochondrial DNA (mtDNA), while the remainder are nuclear-encoded, and imported into the mitochondria.

As used herein a “mitochondrial disease or disorder” refers to any illness resulting from a deficiency of any mitochondrial-located protein which is involved in energy metabolism. Therefore, deficiencies of the respiratory (electron transport) chain, either resulting from a deficiency in none or more of the mitochondrial or nuclear-encoded proteins, are mitochondrial disorders. Also, by definition, disorders of the fatty acid (beta) oxidation, Krebs cycle and pyruvate dehydrogenase complex deficiency are mitochondrial disorders. Although theses disorders may be genetically dissimilar, all disorders contemplated by the present invention are similar in that they result in an energy deficient state.

There is no one identifying feature of mitochondrial disease. Subjects can have combinations of problems whose onset may occur from before birth to late adult life. Mitochondrial diseases should be considered in the differential diagnosis when there are these unexplained features, especially when these occur in combination. Mitochondria disease and disorders can affect multiple organs, resulting in a vast array of symptoms. Symptoms which may affect the brain include, developmental delays, mental retardation, dementia, seizures, neuro-psychiatric disturbances, atypical cerebral palsy, migraines, strokes.

Symptoms which affect the nervous system may include, weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems.

Symptoms which affect muscle may include, weakness, hypotonia, cramping and muscle pain.

Symptoms which affect the kidneys include proximal renal tubular wasting resulting in loss of protein, magnesium, phosphorous, calcium and other electrolytes.

Symptoms which affect the heart include cardiac conduction defects heart blocks) and cardio myopathy.

Symptoms which affect the liver include hypoglycemia (low blood sugar) and liver failure.

Symptoms which affect the eyes include visual loss and blindness.

Symptoms which affect the ears include hearing loss and deafness.

Symptoms which affect the pancreas include diabetes and exocrine pancreatic failure (inability to make digestive enzymes).

There may also be systemic problems associated with mitochondrial dysfunction, including failure to gain weight, short stature, fatigue, respiratory problems

Mitochondrial defects have been linked to Alzheimer's, Parkinson's, diabetes, autism, and the aging process. Other disease associated with mitochondrial dysfunction include, LIC (Lethal Infantile Cardio myopathy), Beta-oxidation Defects, COX Deficiency, Mitochondrial Cytopathy, Alpers Disease, Barth syndrome, Carnitine-Acyl-Carnitine Deficiency, Carnitine Deficiency, Co-Enzyme Q10 Deficiency, Complex I Deficiency, Complex II Deficiency, Complex III Deficiency, Complex IV Deficiency, Complex V Deficiency, CPEO, CPT I Deficiency, Glutaric Aciduria Type II, KSS, lactic acidosis, LCAD, LCHAD, Leigh Disease, LHON, Luft Disease, MAD, MCA, MELAS, MERRF, mitochondrial DNA depletion, Mitochondrial Encephalopath, MNGIE, NARP, Pearson Syndrome, Pyruvate Carboxylase Deficiency, Pyruvate Dehydrogenase Deficiency, SCAD, SCHAD and VLCAD.

Alpers Disease, or Progressive Infantile Poliodystrophy, includes symptoms such as seizures, dementia, spasticity, blindness, liver dysfunction, and cerebral degeneration. (Luft; The development of mitochondrial medicine. Proceedings of the National Academy of Sciences of the United States of America; 1994; 91(19); 8731-8).

Barth syndrome or LIC (Lethal Infantile Cardio myopathy) is an X-linked recessive disorder the symptoms of which include skeletal myopathy, cardio myopathy, short stature, and neutropenia. (Christodoulou; Barth syndrome: clinical observations and genetic linkage studies. American Journal of Medical Genetics; 1994; 50(3); 255-64).

Carnitine-Acyl-Carnitine Deficiency is an autosomal recessive disorder, the symptoms of which are seizures, apnea, bradycardia, vomiting, lethargy, coma, enlarged liver, limb weakness, myoglobin in the urine, Reye-like symptoms triggered by fasting.

Carnitine Deficiency is an autosomal recessive disease, the symptoms of which include Cardio myopathy, failure to thrive, and altered consciousness or coma, sometimes hypotonia.

Co-Enzyme Q10 Deficiency is most likely an autosomal recessive disease, the symptoms of which include Encephalo myopathy, mental retardation, exercise intolerance, ragged-red fibers, and recurrent myoglobin in the urine.

Complex I Deficiency or NADH dehydrogenase (NADH-CoQ reductase) deficiency is an autosomal disease, the symptoms of which are classified by three major forms: (1) fatal infantile multisystem disorder, characterized by developmental delay, muscle weakness, heart disease, congenital lactic acidosis, and respiratory failure; (2) myopathy beginning in childhood or in adult life, manifesting as exercise intolerance or weakness. Elevated lactic acid common; and (3) mitochondrial encephalo myopathy (including MELAS), which may begin in childhood or adult life and consists of variable combinations of symptoms and signs, including ophthalmoplegia, seizures, dementia, ataxia, hearing loss, pigmentary retinopathy, sensory neuropathy, and uncontrollable movements. In addition, this disorder may cause Leigh Syndrome.

Complex II Deficiency or Succinate dehydrogenase deficiency, the symptoms of which include encephalo myopathy and various manifestations, including failure to thrive, developmental delay, hyoptonia, lethargy, respiratory failure, ataxia, myoclonus and lactic acidosis. May also cause Leigh Syndrome.

Complex III Deficiency or Ubiquinone-cytochrome c oxidoreductase deficiency, symptoms of which are categorized in four major forms: (1) fatal infantile encephal myopathy, congenital lactic acidosis, hypotonia, dystrophic posturing, seizures, and coma. Ragged-red fibers common; (2) encephalomyopathies of later onset (childhood to adult life): various combinations of weakness, short stature, ataxia, dementia, hearing loss, sensory neuropathy, pigmentary retinopathy, and pyramidal signs. Ragged-red fibers common. Possible lactic acidosis; (3) myopathy, with exercise intolerance evolving into fixed weakness. Ragged-red fibers common. Possible lactic acidosis; and (4) infantile histiocytoid cardio myopathy.

Complex IV Deficiency or Cytochrome c oxidase deficiency is caused by a defect in Complex IV of the respiratory chain, the symptoms of which can be categorized in two major forms: (1) encephalo myopathy, which is typically normal for the first 6 to 12 months of life and then show developmental regression, ataxia, lactic acidosis, optic atrophy, ophthalmoplegia, nystagmus, dystonia, pyramidal signs, respiratory problems and frequent seizures; and (2) myopathy: Two main variants: (a) Fatal infantile myopathy: may begin soon after birth and accompanied by hypotonia, weakness, lactic acidosis, ragged-red fibers, respiratory failure, and kidney problems: and b) Benign infantile myopathy: may begin soon after birth and accompanied by hypotonia, weakness, lactic acidosis, ragged-red fibers, respiratory problems, but (if the child survives) followed by spontaneous improvement.

Complex V Deficiency or ATP synthase deficiency includes symptoms such as slow, progressive myopathy.

CPEO or Chronic Progressive External Ophthalmoplegia Syndrome includes symptoms such as visual myopathy, retinitis pigmentosa, dysfunction of the central nervous system. It is caused by single mitochondrial DNA deletions, with Mitochondrial DNA point mutation, A3243G being the most common (Luft; The development of mitochondrial medicine. [Review]; Proceedings of the National Academy of Sciences of the United States of America; 1994; 91(19); 8731-8).

CPT I Deficiency is an autosomal recessive disease and includes symptoms such as enlarged liver and recurrent Reye-like episodes triggered by fasting or illnesses.

CPT II Deficiency is an autosomal recessive disease, the symptoms of which include exercise intolerance, fasting intolerance, muscle pain, muscle stiffness, and myoglobin in the urine and in infants, Reye-like syndrome, enlarged liver, hypoglycemia, enlarged heart and cardiac arrhythmia.

KSS or Kearns-Sayre Syndrome, in most cases is caused by large mitochondria DNA deletions. Symptoms associated with KSS include progressive external ophthalmoplegia, pigmentary retinopathy, heart block, and high cerebrospinal protein.

Lactic Acidosis is associated with the accumulation of lactic acid due to its production exceeding its use. Chronic lactic acidosis is a common symptom of mitochondrial disease.

LCAD or Long-Chain Acyl-CoA Dehydrongenase Deficiency, is an autosomal recessive disorder, which causes a fatal syndrome, in infants, typified by failure to thrive, enlarged liver, enlarged heart, metabolic encephalopathy and hypotonia.

LCHAD is an autosomal recessive disorder, characterized by symptoms such as encephalopathy, liver dysfunction, cardio myopathy, and myopathy. Also pigmentary retinopathy and peripheral neuropathy.

Leigh Disease or Syndrome or Subacute Necrotizing Encephalomyelopathy is characterized by symptoms such as Seizures, hypotonia, fatigue, nystagmus, poor reflexes, eating and swallowing difficulties, breathing problem and poor motor function,

LHON or Leber Hereditary Optic Neuropathy is caused by mitochondrial DNA point mutations, including G14459A, among others. Symptoms associated with LHON include primarily blindness in young men. Less common symptoms include mild dementia, ataxia, spasticity, peripheral neuropathy and heart conduction defects.

Luft Disease is characterized by symptoms such as hypermetabolism, with fever, heat intolerance, profuse perspiration, polyphagia, polydipsia, ragged-red fibers, and resting tachycardia. In addition to exercise intolerance with mild weakness.

MAD or Glutaric Aciduria Type II or multiple Acyl-CoA Dehydrogenase Deficiency is caused by defects of the flavoproteins responsible for transferring electrons (ETF or ETF-dehydrogenase) therefor affecting the function of all six ETF-funneling acyl-CoA dehydrogenases.

MCAD or Medium-Chain Acyl-CoA Dehydrongenase Deficiency is an autosomal recessive disorder, which afflicts infants or young children with episodes of encephalopathy, enlarged and fatty degeneration of the liver, and low carnitine in the blood.

MELAS or Mitochondrial Encephalo myopathy Lactic Acidosis and Strokelike Episodes is caused by mitochondrial DNA point mutations, the most common of which is A3243G. It is characterized by symptoms: Short statue, seizures, stroke-like episodes with focused neurological deficits, recurrent headaches, cognitive regression, disease progression ragged-red fibers (Koo, et. al.; Mitochondrial encephalo myopathy, lactic acidosis, stroke-like episodes (MELAS): clinical, radiological, pathological, and genetic observations. Annals of Neurology; 1993 ; 34(1); 25-32).

MERRF or Myoclonic Epilepsy and Ragged-Red Fiber Disease is caused by the mitochondrial DNA point mutations A8344G and T8356C. Its symptoms include myoclonus, epilepsy, progressive ataxia, muscle weakness and degeneration, deafness and dementia (Luft; The development of mitochondrial medicine; Proceedings of the National Academy of Sciences of the United States of America; 1994 ; 91(19); 8731-8).

There are three forms of mitochondrial DNA Depletion. These include: (1) congenital myopathy: Neonatal weakness, hypotonia requiring assisted ventilation, possible renal dysfunction. Severe lactic acidosis. Prominent ragged-red fibers. Death due to respiratory failure usually occurs prior to one year of age; (2) infantile myopathy: Following normal early development until one year old, weakness appears and worsens rapidly, causing respiratory failure and death typically within a few years; and (3) hepatopathy, enlarged liver and intractable liver failure, myopathy. Severe lactic acidosis. Death is typical within the first year.

Mitochondrial Encephalopathy, also includes Encephalo myopathy and Encephalomyelopathy.

MNGIE or Myoneurogastointestinal Disorder and Encephalopathy, include symptoms such as progressive external ophthalmoplegia, limb weakness, peripheral neuropathy, digestive tract disorders, leukodystrophy, lactic acidosis and ragged red fibers.

NARP or Neuropathy, Ataxia, and Retinitis Pigmentosa is caused by mitochondrial DNA point mutations in genes associated with Complex V, including T8993G, (also T8993C by some researchers). Leigh Syndrome may result if the percentage of mutation is high enough.

Pearson Syndrome is characterized by symptoms associated with bone marrow and pancreas dysfunction. It is caused by single mitochondrial DNA deletions. Inheritance is usually sporadic.

Those who survive infancy usually develop Kearns-Sayre Syndrome.

Pyruvate Carboxylase Deficiency is an autosomal recessive disorder, the symptoms of which include lactic acidosis, hypoglycemia, severe retardation, failure to thrive, in addition to seizures and spasticity.

Pyruvate Dehydrogenase Deficiency is characterized by symptoms such as lactic acidosis, ataxia, pyruvic acidosis, spinal and cerebellar degeneration. Less common symptoms include agenesis of the corpus callosum and lesions in the basal ganglia, cerebelum, and brain stem, growth delay, hypotonia, seizures and polyneuropathy.

SCAD or Short-Chain Acyl-CoA Dehydrogenase Deficiency, is an autosomal recessive disorder characterized by symptoms such as failure to thrive, developmental delay and hypoglycemia.

SCHAD is an autosomal recessive disorder, characterized by encephalopathy and possibly liver disease or cardio myopathy.

VLCAD or Very Long-Chain Acyl-CoA Dehydrongenase Deficiency is an autosomal recessive disorder, characterized by various manifestations, ranging from fatal infantile encephalopathy to recurrent myoglobin in the urine, similar to the myopathic form of CPT II deficiency.

In addition, other diseases and disorders which can be treated using the methods of the present invention include, without being limited to, A-Beta-Lipoproteinemia, A-V, A Beta-2-Microglobulin Amyloidosis, A-T, A1AD, A1AT, Aagenaes, Aarskog syndrome, Aarskog-Scott Syndrome, Aase-smith syndrome, Aase Syndrome, AAT, Abderhalden-Kaufmann-Lignac Syndrome, Abdominal Muscle Deficiency Syndrome, Abdominal Wall Defect, Abdominal Epilepsy, Abdominal Migraine, Abductor Spasmodic Dysphonia, Abductor Spastic Dysphonia, Abercrombie Syndrome, blepharon-Macrostomia Syndrome, ABS, Absence of HPRT, Absence of Corpus Callosum Schinzel Typ, Absence Defect of Limbs Scalp and Skull, Absence of Menstruation Primar, Absence of HGPRT, Absorptive Hyperoxaluriaor Enteric, Abt-Letterer-Siwe Disease, ACADL, ACADM Deficiency, ACADM, ACADS, Acanthocytosis-Neurologic Disorder, Acanthocytosis, Acantholysis Bullosa, Acanthosis Nigricans, Acanthosis Bullosa, Acanthosis Nigricans With Insulin Resistance Type A, Acanthosis Nigricans With Insulin Resistance Type B, Acanthotic Nevus, Acatalasemia, Acatalasia, ACC, Accessory Atrioventricular Pathways, Accessory Atrioventricular Pathways, Acephaly, ACF with Cardiac Defects, Achalasia, Achard-Thiers Syndrome, ACHARD (Marfan variant), Achard's syndrome, Acholuric Jaundice, Achondrogenesis, Achondrogenesis Type IV, Achondrogenesis Type III, Achondroplasia, Achondroplasia Tarda, Achondroplastic Dwarfism, Achoo Syndrome, Achromat, Achromatope, Achromatopic, Achromatopsia, Achromic Nevi, Acid Ceramidase Deficiency, Acid Maltase Deficiency, Acid Beta-glucosidase Deficiency, Acidemia Methylmalonic, Acidemia Propionic, Acidemia with Episodic Ataxia and Weakness, Acidosis, Aclasis Tarsoepiphyseal, ACM, Acoustic Neurilemoma, Acoustic Neuroma, ACPS with Leg Hypoplasia, ACPS II, ACPS IV, ACPS III, Acquired Aphasia with Convulsive Disorder, Acquired Brown Syndrome, Acquired Epileptic Aphasia, Acquired Factor XIII Deficiency, Acquired Form of ACC (caused by infection while still in womb), Acquired Hyperoxaluria, Acquired Hypogammaglobulinemia, Acquired Immunodeficiency Syndrome (AIDS), Acquired Iron Overload, Acquired Lipodystrophy, Acquired Partial Lipodystrophy, Acquired Wandering Spleen, ACR, Acral Dysostosis with Facial and Genital Abnormalities, Acro Renal, Acrocallosal Syndrome Schinzel Type, Acrocephalosyndactyly, Acrocephalosyndactyly Type I, Acrocephalosyndactyly Type I Subtype I, Acrocephalopolysyndactyly Type II, Acrocephalopolysyndactyly Type III, Acrocephalopolysyndactyly Type IV, Acrocephalosyndactyly V (ACS5 or ACS V) Subtype I, Acrocephaly Skull Asymmetry and Mild Syndactyly, Acrocephaly, Acrochondrohyperplasia, Acrodermatitis Enteropathica, Acrodysostosis, Acrodystrophic Neuropathy, Acrofacial Dysostosis Nager Type, Acrofacial Dysostosis Postaxial Type, Acrofacial Dysostosis Type Genee-Wiedep, Acrogeria Familial, Acromegaly, Acromelalgia Hereditary, Acromesomelic Dysplasia, Acromesomelic Dwarfism, Acromicric Skeletal Dysplasia, Acromicric Dysplasia, Acroosteolysis with Osteoporosis and Changes in Skull and Mandible, Acroosteolysis, Acroparesthesia, ACS I, ACS Type II, ACS Type III, ACS, ACS3, ACTH Deficiency, Action Myoclonus, Acute Brachial Neuritis Syndrome, Acute Brachial Radiculitis Syndrome, Acute Cerebral Gaucher Disease, Acute Cholangitis, Acute Disseminated Encephalomyeloradiculopathy, Acute Disseminated Histiocytosis-X, Acute Hemorrhagic Polioencephalitis, Acute Idiopathic Polyneuritis, Acute Immune-Mediation Polyneuritis, Acute Infantile Pelizaeus-Merzbacher Brain Sclerosis, Acute Intermittant Porphyria, Acute Porphyrias, Acute Sarcoidosis, Acute Shoulder Neuritis, Acute Toxic Epidermolysis, Acyl-CoA Dehydrogenase Deficiency Long-Chain, Acyl-CoA Dehydrogenase Deficiency Short-Chain, Acyl-CoA Dihydroxyacetone Acyltransferase, Acyl-coenzyme A Oxidase Deficiency, ADA, ADA Deficiency, Adam Complex, Adamantiades-Behcet's Syndrome, Adamantinoma, Adams Oliver Syndrome, Adaptive Colitis, ADD combined type, ADD, Addison Disease with Cerebral Sclerosis, Addison's Anemia, Addison's Disease, Addison-Biermer Anemia, Addison-Schilder Disease, Addisonian Pernicious Anemia, Adducted Thumbs-Mental Retardation, Adductor Spasmodic Dysphonia, Adductor Spastic Dysphonia, Adenoma Associated Virilism of Older Women, Adenomatosis of the Colon and Rectum, Adenomatous polyposis of the Colon, Adenomatous Polyposis Familial, Adenosine Deaminase Deficiency, Adenylosuccinase deficiency, ADHD predominantly hyperactive-impulsive type, ADHD predominantly inattentive type, ADHD, Adhesive Arachnoiditis, Adie Syndrome, Adie's Syndrome, Adie's Tonic Pupil, Adie's Pupil, Adipogenital Retinitis Pigmentosa Polydactyly, Adipogenital-Retinitis Pigmentosa Syndrome, Adiposa Dolorosa, Adiposis Dolorosa, Adiposogenital Dystrophy, Adolescent Cystinosis, ADPKD, Adrenal Cortex Adenoma, Adrenal Disease, Adrenal Hyperfunction resulting from Pituitary ACTH Excess, Adrenal Hypoplasia, Adrenal Insufficiency, Adrenal Neoplasm, Adrenal Virilism, Adreno-Retinitis Pigmentosa-Polydactyly Syndrome, Adrenocortical Insufficiency, Adrenocortical Hypofunction, Adrenocorticotropic Hormone Deficiency Isolated, Adrenogenital Syndrome, Adrenoleukodystrophy, Adrenomyeloneuropathy, Adreno-Retinitis Pigmentosa-Polydactyly Syndrome, Adult Cystinosis, Adult Dermatomyositis, Adult Hypophosphatasia, Adult Macula Lutea Retinae Degeneration, Adult Onset ALD, Adult-Onset Ceroidosis, Adult Onset Medullary Cystic Disease, Adult Onset Pernicious Anemia, Adult Onset Schindler Disease, Adult-Onset Subacute Necrotizing Encephalomyelopathy, Adult Polycystic Kidney Disease, Adult Onset Medullary Cystic Disease, Adynlosuccinate Lyase Deficiency, AE, AEC Syndrome, AFD, Afibrinogenemia, African Siderosis, AGA, Aganglionic Megacolon, Age Related Macular Degeneration, Agenesis of Commissura Magna Cerebri, Agenesis of Corpus Callosum, Agenesis of Corpus Callosum-Infantile Spasms-Ocular Anomalies, Agenesis of Corpus Callosum and Chorioretinal Abnormality, Agenesis of Corpus Callosum-Chorioretinitis Abnormality, Aggressive mastocytosis, Agnosis Primary, AGR Triad, AGU, Agyria, Agyria-pachygria-band spectrum, AHC, AHD, AHDS, AHF Deficiency, AHG Deficiency, AHO, Ahumada Del Castillo, Aicardi Syndrome, AIED, AIMP, AIP, AIS, Akinetic Seizure, ALA-D Porphyria, Alactasia, Alagille Syndrome, Aland Island Eye Disease (X-Linked), Alaninuria, Albers-Schonberg Disease, Albinism, Albinismus, Albinoidism, Albright Hereditary Osteodystrophy, Alcaptonuria, Alcohol-Related Birth Defects, Alcoholic Embryopathy, Ald, ALD, ALD, Aldosterone, Aldosteronism With Normal Blood Pressure, Aldrich Syndrome, Alexander's Disease, Alexanders Disease, Algodystrophy, Algoneurodystrophy, Alkaptonuria, Alkaptonuric Ochronosis, Alkyl DHAP synthase deficiency, Allan-Herndon-Dudley Syndrome, Allan-Herndon Syndrome, Allan-Hemdon-Dudley Mental Retardation, Allergic Granulomatous Antitis, Allergic Granulomatous Angiitis of Cronkhite-Canada, Alobar Holoprosencephaly, Alopecia Areata, Alopecia Celsi, Alopecia Cicatrisata, Alopecia Circumscripta, Alopecia-Poliosis-Uveitis-Vitiligo-Deafness-Cutaneous-Uveo-O, Alopecia Seminuniversalis, Alopecia Totalis, Alopecia Universalis, Alpers Disease, Alpers Diff-use Degeneration of Cerebral Gray Matter with Hepatic Cirrhosis, Alpers Progressive Infantile Poliodystrophy, Alpha-1-Antitrypsin Deficiency, Alpha-1 4 Glucosidase Deficiency, Alpha-Galactosidase A Deficiency, Alpha-Galactosidase B Deficiency, Alpha High-Density Lipoprotein Deficieny, Alpha-L-Fucosidase Deficiency Fucosidosis Type 3, Alpha-GalNAc Deficiency Schindler Type, Alphalipoproteinemia, Alpha Mannosidosis, Alpha-N-Acetylgalactosaminidase Deficiency Schindler Type, Alpha-NAGA Deficiency Schindler Type, Alpha-Neuraminidase Deficiency, Alpha-Thalassemia/mental retardation syndrome non-deletion type, Alphalipoproteinemia, Alport Syndrome, ALS, Alstroem's Syndrome, Alstroem, Alstrom Syndrome, Alternating Hemiplegia Syndrome, Alternating Hemiplegia of Childhood, Alzheimer's Disease, Amaurotic Familial Idiocy, Amaurotic Familial Idiocy Adult, Amaurotic Familial Infantile Idiocy, Ambiguous Genitalia, AMC, AMD, Ameloblastoma, Amelogenesis Imperfecta, Amenorrhea-Galactorrhea Nonpuerperal, Amenorrhea-Galactorrhea-FSH Decrease Syndrome, Amenorrhea, Amino Acid Disorders, Aminoaciduria-Osteomalacia-Hyperphosphaturia Syndrome, AMN, Amniocentesis, Amniotic Bands, Amniotic Band Syndrome, Amniotic Band Disruption Complex, Amniotic Band Sequence, Amniotic Rupture Sequence, Amputation Congenital, AMS, Amsterdam Dwarf Syndrome de Lange, Amylo-1 6-Glucosidase Deficiency, Amyloid Arthropathy of Chronic Hemodialysis, Amyloid Corneal Dystrophy, Amyloid Polyneuropathy, Amyloidosis, Amyloidosis of Familial Mediterranean Fever, Amylopectinosis, Amyoplasia Congenita, Amyotrophic Lateral Sclerosis, Amyotrophic Lateral Sclerosis, Amyotrophic Lateral Sclerosis-Polyglucosan Bodies, AN, AN 1, AN 2, Anal Atresia, Anal Membrane, Anal Rectal Malformations, Anal Stenosis, Analine 60 Amyloidosis, Analphalipoproteinemia, Analrectal, Analrectal, Anaplastic Astrocytoma, Andersen Disease, Anderson-Fabry Disease, Andersen Glycogenosis, Anderson-Warburg Syndrome, Andre Syndrome, Andre Syndrome Type II, Androgen Insensitivity, Androgen Insensitivity Syndrome Partial, Androgen Insensitivity Syndrome Partial, Androgenic Steroids, Anemia Autoimmune Hemolytic, Anemia Blackfan Diamond, Anemia, Congenital, Triphalangeal Thumb Syndrome, Anemia Hemolytic Cold Antibody, Anemia Hemolytic with PGK Deficiency, Anemia Pernicious, Anencephaly, Angelman Syndrome, Angio-Osteohypertrophy Syndrome, Angiofollicular Lymph Node Hyperplasia, Angiohemophilia, Angiokeratoma Corporis, Angiokeratoma Corporis Difflisum, Angiokeratoma Diffuse, Angiomatosis Retina, Angiomatous Lymphoid, Angioneurotic Edema Hereditary, Anhidrotic Ectodermal Dysplasia, Anhidrotic X-Linked Ectodermal Dysplasias, Aniridia, Aniridia-Ambiguous Genitalia-Mental Retardation, Aniridia Associated with Mental Retardation, Aniridia-Cerebellar Ataxia-Mental Deficiency, Aniridia Partial-Cerebellar Ataxia-Mental Retardation, Aniridia Partial-Cerebellar Ataxia-Oligophrenia, Aniridia Type I, Aniridia Type II, Aniridia-Wilms' Tumor Association, Aniridia-Wilms' Tumor-Gonadoblastoma, Ankyloblepharon-Ectodermal Defects-Cleft Lip/Palate, Ankylosing Spondylitis, Annular groves, Anodontia, Anodontia Vera, Anomalous Trichromasy, Anomalous Dysplasia of Dentin, Coronal Dentin Dysplasia, Anomic Aphasia, Anophthalmia, Anorectal, Anorectal Malformations, Anosmia, Anterior Bowing of the Legs with Dwarfism, Anterior Membrane Corneal Dystrophy, Anti-Convulsant Syndrome, Anti-Epstein-Barr Virus Nuclear Antigen (EBNA) Antibody Deficiency, Antibody Deficiency, Antibody Deficiency with near normal Immunoglobulins, Antihemophilic Factor Deficiency, Antihemophilic Globulin Deficiency, Antiphospholipid Syndrome, Antiphospholipid Antibody Syndrome, Antithrombin III Deficiency, Antithrombin III Deficiency Classical (Type I), Antitrypsin Deficiency, Antley-Bixler Syndrome, Antoni's Palsy, Anxietas Tibialis, Aorta Arch Syndrome, Aortic and Mitral Atresia with Hypoplasic Left Heart Syndrome, Aortic Stenosis, Aparoschisis, APC, APECED Syndrome, Apert Syndrome, Aperts, Aphasia, Aplasia Axialis Extracorticales Congenital, Aplasia Cutis Congenita, Aplasia Cutis Congenita with Terminal Transverse Limb Defects, Aplastic Anemia, Aplastic Anemia with Congenital Anomalies, APLS, Apnea, Appalachian Type Amyloidosis, Apple Peel Syndrome, Apraxia, Apraxia Buccofacial, Apraxia Constructional, Apraxia Ideational, Apraxia Ideokinetic, Apraxia Ideomotor, Apraxia Motor, Apraxia Oculomotor, APS, Arachnitis, Arachnodactyly Contractural Beals Type, Arachnodactyly, Arachnoid Cysts, Arachnoiditis Ossificans, Arachnoiditis, Aran-Duchenne, Aran-Duchenne Muscular Atrophy, Aregenerative Anemia, Arginase Deficiency, Argininemia, Arginino Succinase Deficiency, Argininosuccinase Deficiency, Argininosuccinate Lyase Deficiency, Argininosuccinic Acid Lyase-ASL, Argininosuccinic Acid Synthetase Deficiency, Argininosuccinic Aciduria, Argonz-Del Castillo Syndrome, Arhinencephaly, Armenian Syndrome, Arnold-Chiari Malformation, Arnold-Chiari Syndrome, ARPKD, Arrhythmic Myoclonus, Arrhythmogenic Right Ventricular Dysplasia, Arteriohepatic Dysplasia, Arteriovenous Malformation, Arteriovenous Malformation of the Brain, Arteritis Giant Cell, Arthritis, Arthritis Urethritica, Arthro-Dento-Osteodysplasia, Arthro-Ophthalmopathy, Arthrochalasis Multiplex Congenita, Arthrogryposis Multiplex Congenita, Arthrogryposis Multiplex Congenita, Distal, Type IIA, ARVD, Arylsulfatase-B Deficiency, AS, ASA Deficiency, Ascending Paralysis, ASD, Atrioseptal Defects, ASH, Ashermans Syndrome, Ashkenazi Type Amyloidosis, ASL Deficiency, Aspartylglucosarninuria, Aspartylglycosaminuria, Asperger's Syndrome, Asperger's Type Autism, Asphyxiating Thoracic Dysplasia, Asplenia Syndrome, ASS Deficiency, Asthma, Astrocytoma Grade I (Benign), Astrocytoma Grade II (Benign), Asymmetric Crying Facies with Cardiac Defects, Asymmetrical septal hypertrophy, Asymptomatic Callosal Agenesis, AT, AT Im Deficiency, AT III Variant IA, AT III Variant lb, AT 3, Ataxia, Ataxia Telangiectasia, Ataxia with Lactic Acidosis Type II, Ataxia Cerebral Palsy, Ataxiadynamia, Ataxiophemia, ATD, Athetoid Cerebral Palsy, Atopic Eczema, Atresia of Esophagus with or without Tracheoesophageal Fistula, Atrial Septal Defects, Atrial Septal Defect Primum, Atrial and Septal and Small Ventricular Septal Defect, Atrial Flutter, Atrial Fibrillation, Atriodigital Dysplasia, Atrioseptal Defects, Atrioventricular Block, Atrioventricular Canal Defect, Atrioventricular Septal Defect, Atrophia Bulborum Hereditaria, Atrophic Beriberi, Atrophy Olivopontocerebellar, Attention Deficit Disorder, Attention Deficit Hyperactivity Disorder, Attentuated Adenomatous Polyposis Coli, Atypical Amyloidosis, Atypical Hyperphenylalaninemia, Auditory Canal Atresia, Auriculotemporal Syndrome, Autism, Autism Asperger's Type, Autism Dementia Ataxia and Loss of Purposeful Hand Use, Autism Infantile Autism, Autoimmune Addison's Disease, Autoimmune Hemolytic Anemia, Autoimmune Hepatitis, Autoimmune-Polyendocrinopathy-Candidias, Autoimmune Polyglandular Disease Type I, Autosomal Dominant Albinism, Autosomal Dominant Compelling Helioophthalmic Outburst Syndrome, Autosomal Dominant Desmin Distal myopathy with Late Onset, Autosomal Dominant EDS, Au tosomal Dominant Emery-Dreifuss Muscular Dystrophy, Autosomal Dominant Keratoconus, Autosomal Dominant Pelizaeus-Merzbacher Brain Sclerosis, Autosomal Dominant Polycystic Kidney Disease, Autosomal Dominant Spinocerebellar Degeneration, Autosomal Recessive Agammaglobulinemia, Autosomal Recessive Centronuclear myopathy, Autosomal Recessive Conradi-Hunermann Syndrome, Autosomal Recessive EDS, Autosomal Recessive Emery-Dreifuss Muscular Dystrophy, Autosomal Recessive Forms of Ocular Albinism, Autosomal Recessive Inheritance Agenesis of Corpus Callosum, Autosomal Recessive Keratoconus, Autosomal Recessive Polycystic Kidney Disease, Autosomal Recessive Severe Combined Immunodeficiency, AV, AVM, AVSD, AWTA, Axilla Abscess, Axonal Neuropathy Giant, Azorean Neurologic Disease, B-K Mole Syndrome, Babinski-Froelich Syndrome, BADS, Baillarger's Syndrome, Balkan Disease, Baller-Gerold Syndrome, Ballooning Mitral Valve, Balo Disease Concentric Sclerosis, Baltic Myoclonus Epilepsy, Bannayan-Zonana syndrome (BZS), Bannayan-Riley-Ruvalcaba syndrome, Banti's Disease, Bardet-Biedl Syndrome, Bare Lymphocyte Syndrome, Barlow's syndrome, Barraquer-Simons Disease, Barrett Esophagus, Barrett Ulcer, Barth Syndrome, Bartter's Syndrome, Basal Cell Nevus Syndrome, Basedow Disease, Bassen-Komzweig Syndrome, Batten Disease, Batten-Mayou Syndrome, Batten-Spielmeyer-Vogt's Disease, Batten Turner Syndrome, Batten Turner Type Congenital myopathy, Batten-Vogt Syndrome, BBB Syndrome, BBB Syndrome (Opitz), BBB Syndrome, BBBG Syndrome, BCKD Deficiency, BD, BDLS, BE, Beals Syndrome, Beals Syndrome, Beals-Hecht Syndrome, Bean Syndrome, BEB, Bechterew Syndrome, Becker Disease, Becker Muscular Dystrophy, Becker Nevus, Beckwith Wiedemann Syndrome, Beckwith-Syndrome, Begnez-Cesar's Syndrome, Behcet's syndrome, Behcet's Disease, Behr 1, Behr 2, Bell's Palsy, Benign Acanthosis Nigricans, Benign Astrocytoma, Benign Cranial Nerve Tumors, Benign Cystinosis, Benign Essential Blepharospasm, Benign Essential Tremor, Benign Familial Hematuria, Benign Focal Amyotrophy, Benign Focal Amyotrophy of ALS, Benign Hydrocephalus, Benign Hypermobility Syndrome, Benign Keratosis Nigricans, Benign Paroxysmal Peritonitis, Benign Recurrent Hematuria, Benign Recurrent Intrahepatic Cholestasis, Benign Spinal Muscular Atrophy with Hypertrophy of the Calves, Benign Symmetrical Lipomatosis, Benign Tumors of the Central Nervous System, Berardinelli-Seip Syndrome, Berger's Disease, Beriberi, Berman Syndrome, Bernard-Homer Syndrome, Bernard-Soulier Syndrome, Besnier Prurigo, Best Disease, Beta-Alanine-Pyruvate Aminotransferase, Beta-Galactosidase Deficiency Morquio Syndrome, Beta-Glucuronidase Deficiency, Beta Oxidation Defects, Beta Thalassemia Major, Beta Thalassemia Minor, Betalipoprotein Deficiency, Bethlem myopathy, Beuren Syndrome, BH4 Deficiency, Biber-Haab-Dimmer Corneal Dystrophy, Bicuspid Aortic Valve, Biedl-Bardet, Bifid Cranium, Bifunctional Enzyme Deficiency, Bilateral Acoustic Neurofibromatosis, Bilateral Acoustic Neuroma, Bilateral Right-Sidedness Sequence, Bilateral Renal Agenesis, Bilateral Temporal Lobe Disorder, Bilious Attacks, Bilirubin Glucuronosyltransferase Deficiency Type I, Binder Syndrome, Binswanger's Disease, Binswanger's Encephalopathy, Biotinidase deficiency, Bird-Headed Dwarfism Seckel Type, Birth Defects, Birthmark, Bitemporal Forceps Marks Syndrome, Biventricular Fibrosis, Bjornstad Syndrome, B-K Mole Syndrome, Black Locks-Albinism-Deafness of Sensoneural Type (BADS), Blackfan-Diamond Anemia, Blennorrheal Idiopathic Arthritis, Blepharophimosis, Ptosis, Epicanthus Inversus Syndrome, Blepharospasm, Blepharospasm Benign Essential, Blepharospasm Oromandibular Dystonia, Blessig Cysts, BLFS, Blindness, Bloch-Siemens Incontinentia Pigmenti Melanoblastosis Cutis Linearis, Bloch-Siemens-Sulzberger Syndrome, Bloch-Sulzberger Syndrome, Blood types, Blood type A, Blood type B, Blood type AB, Blood type O, Bloom Syndrome, Bloom-Torre-Mackacek Syndrome, Blue Rubber Bleb Nevus, Blue Baby, Blue Diaper Syndrome, BMD, BOD, BOFS, Bone Tumor-Epidermoid Cyst-Polyposis, Bonnet-Dechaume-Blanc Syndrome, Bonnevie-Ulrich Syndrome, Book Syndrome, BOR Syndrome, BORJ, Borjeson Syndrome, Borjeson-Forssman-Lehmann Syndrome, Bowen Syndrome, Bowen-Conradi Syndrome, Bowen-Conradi Hutterite, Bowen-Conradi Type Hutterite Syndrome, Bowman's Layer, BPEI, BPES, Brachial Neuritis, Brachial Neuritis Syndrome, Brachial Plexus Neuritis, Brachial-Plexus-Neuropathy, Brachiocephalic Ischemia, Brachmann-de Lange Syndrome, Brachycephaly, Brachymorphic Type Congenital, Bradycardia, Brain Tumors, Brain Tumors Benign, Brain Tumors Malignant, Branched Chain Alpha-Ketoacid Dehydrogenase Deficiency, Branched Chain Ketonuria I, Brancher Deficiency, Branchio-Oculo-Facial Syndrome, Branchio-Oto-Renal Dysplasia, Branchio-Oto-Renal Syndrome, Branchiooculofacial Syndrome, Branchiootic Syndrome, Brandt Syndrome, Brandywine Type Dentinogenesis Imperfecta, Brandywine type Dentinogenesis Imperfecta, Breast Cancer, BRIC Syndrome, Brittle Bone Disease, Broad Beta Disease, Broad Thumb Syndrome, Broad Thumbs and Great Toes Characteristic Facies and Mental Retardation, Broad Thumb-Hallux, Broca's Aphasia, Brocq-Duhring Disease, Bronze Diabetes, Bronze Schilder's Disease, Brown Albinism, Brown Enamel Hereditary, Brown-Sequard Syndrome, Brown Syndrome, BRRS, Brueghel Syndrome, Bruton's Agammaglobulinemia Common, BS, BSS, Buchanan's Syndrome, Budd's Syndrome, Budd-Chiari Syndrome, Buerger-Gruetz Syndrome, Bulbospinal Muscular Atrophy-X-linked, Bulldog Syndrome, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, BWS, Byler's Disease, C Syndrome, C1 Esterase Inhibitor Dysfunction Type II Angioedema, C1-INH, C1 Esterase Inhibitor Deficiency Type I Angioedema, CINH, Cacchi-Ricci Disease, CAD, CADASIL, CAH, Calcaneal Valgus, Calcaneovalgus, Calcium Pyrophosphate Dihydrate Deposits, Callosal Agenesis and Ocular Abnormalities, Calves-Hypertrophy of Spinal Muscular Atrophy, Campomelic Dysplasia, Campomelic Dwarfism, Campomelic Syndrome, Camptodactyly-Cleft Palate-Clubfoot, Camptodactyly-Limited Jaw Excursion, Camptomelic Dwarfism, Camptomelic Syndrome, Camptomelic Syndrome Long-Limb Type, Camurati-Engelmann Disease, Canada-Cronkhite Disease, Canavan disease, Canavan's Disease Included, Canavan's Leukodystrophy, Cancer, Cancer Family Syndrome Lynch Type, Cantrell Syndrome, Cantrell-Haller-Ravich Syndrome, Cantrell Pentalogy, Carbamyl Phosphate Synthetase Deficiency, Carbohydrate Deficient Glycoprotein Syndrome, Carbohydrate-Deficient Glycoprotein Syndrome Type Ia, Carbohydrate-Induced Hyperlipemia, Carbohydrate Intolerance of Glucose Galactose, Carbon Dioxide Acidosis, Carboxylase Deficiency Multiple, Cardiac-Limb Syndrome, Cardio-auditory Syndrome, Cardioauditory Syndrome of Jervell and and Lange-Nielsen, Cardiocutaneous Syndrome, Cardio-facial-cutaneous syndrome, Cardiofacial Syndrome Cayler Type, Cardiomegalia Glycogenica Diffusa, Cardiomyopathic Lentiginosis, Cardio myopathy, Cardio myopathy Associated with Desmin Storage myopathy, Cardio myopathy Due to Desmin Defect, Cardio myopathy-Neutropenia Syndrome, Cardio myopathy-Neutropenia Syndrome Lethal Infantile Cardio myopathy, Cardiopathic Amyloidosis, Cardiospasm, Cardocardiac Syndrome, Caruitine-Acylcarnitine Translocase Deficiency, Carnitine Deficiency and Disorders, Carnitine Deficiency Primary, Carnitine Deficiency Secondary, Carnitine Deficiency Secondary to MCAD Deficiency, Carnitine Deficiency Syndrome, Carnitine Pahnitoyl Transferase I & II (CPT I & II), Carnitine Palmitoyltransferase Deficiency, Carnitine Palmitoyltransferase Deficiency Type 1, Carnitine Palmitoyltransferase Deficiency Type 2 benign classical muscular form included severe infantile form included, Carnitine Transport Defect (Primary Carnitine Deficiency), Carnosinase Deficiency, Carnosinemia, Caroli Disease, Carpenter syndrome, Carpenter's, Cartilage-Hair Hypoplasia, Castleman's Disease, Castleman's Disease Hyaline Vascular Type, Castleman's Disease Plasma Cell Type, Castleman Tumor, Cat Eye Syndrome, Cat's Cry Syndrome, Catalayse deficiency, Cataract-Dental Syndrome, Cataract X-Linked with Hutchinsonian Teeth, Catecholamine hormones, Catel-Manzke Syndrome, Catel-Manzke Type Palatodigital Syndrome, Caudal Dysplasia, Caudal Dysplasia Sequence, Caudal Regression Syndrome, Causalgia Syndrome Major, Cavernomas, Cavernous Angioma, Cavernous Hemangioma, Cavernous Lymphangioma, Cavernous Malformations, Cayler Syndrome, Cazenave's Vitiligo, CBGD, CBPS, CCA, CCD, CCHS, CCM Syndrome, CCMS, CCO, CD, CDG1a, CDG1A, CDGS Type Ia, CDGS, CDI, CdLS, Celiac Disease, Celiac sprue, Celiac Sprue-Dermatitis, Cellular Immunodeficiency with Purine Nucleoside Phosphorylase Deficiency, Celsus' Vitiligo, Central Apnea, Central Core Disease, Central Diabetes Insipidus, Central Form Neurofibromatosis, Central Hypoventilation, Central Sleep Apnea, Centrifugal Lipodystrophy, Centronuclear myopathy, CEP, Cephalocele, Cephalothoracic Lipodystrophy, Ceramide Trihexosidase Deficiency, Cerebellar Agenesis, Cerebellar Aplasia, Cerebellar Hemiagenesis, Cerebellar Hypoplasia, Cerebellar Vermis Aplasia, Cerebellar Vermis Agenesis-Hypernea-Episodic Eye Moves-Ataxia-Retardation, Cerebellar Syndrome, Cerebellarparenchymal Disorder IV, Cerebellomedullary Malformation Syndrome, Cerebello-Oculocutaneous Telangiectasia, Cerebelloparenchymal Disorder IV Familial, Cerebellopontine Angle Tumor, Cerebral Arachnoiditis, Cerebral Autosomal Dominant Arteriopathy with Subcortical Infarcts and Leukodystrophy, Cerebral Beriberi, Cerebral Diplegia, Cerebral Gigantism, Cerebral Malformations Vascular, Cerebral Palsy, Cerebro-Oculorenal Dystrophy, Cerebro-Oculo-Facio-Skeletal Syndrome, Cerebrocostomandibular syndrome, Cerebrohepatorenal Syndrome, Cerebromacular Degeneration, Cerebromuscular Dystrophy Fukuyama Type, Cerebroocular Dysgenesis, Cerebroocular Dysplasia-Muscular Dystrophy Syndrome, Cerebrooculofacioskeletal Syndrome, Cerebroretinal Arteriovenous Aneurysm, Cerebroside Lipidosis, Cerebrosidosis, Cerebrotendinous Xanthomatosis, Cerebrovascular Ferrocalcinosis, Ceroid-Lipofuscinosis Adult form, Cervical Dystonia, Cervical Dystonia, Cervico-Oculo-Acoustic Syndrome, Cervical Spinal Stenosis, Cervical Vertebral Fusion, CES, CF, CFC syndrome, CFIDS, CFND, CGD, CGF, Chalasodermia Generalized, Chanarin Dorfinan Disease, Chanarin Dorfman Syndrome, Chanarin Dorfinan Ichthyosis Syndrome, Chandler's Syndrome, Charcot's Disease, Charcot-Marie-Tooth, Charcot-Marie-Tooth Disease, Charcot-Marie-Tooth Disease Variant, Charcot-Marie-Tooth-Roussy-Levy Disease, CHARGE Association, Charge Syndrome, CHARGE Syndrome, Chaund's Ectodermal Dysplasias, Chediak-Higashi Syndrome, Chediak-Steinbrinck-Higashi Syndrome, Cheilitis Granulomatosa, Cheiloschisis, Chemke Syndrome, Cheney Syndrome, Cherry Red Spot and Myoclonus Syndrome, CHF, CHH, Chiari's Disease, Chiari Malformation I, Chiari Malformation, Chiari Type I (Chiari Malformation I), Chiari Type II (Chiari Malformation II), Chiari I Syndrome, Chiari-Budd Syndrome, Chiari-Frommel Syndrome, Chiari Malformation II, CHILD Syndrome, CHILD Ichthyosis Syndrome, CHILD Syndrome Ichthyosis, Childhood Adrenoleukodystrophy, Childhood Dermatomyositis, Childhood-onset Dystonia, Childhood Cyclic Vomiting, Childhood Giant Axonal Neuropathy, Childhood Hypophosphatasia, Childhood Muscular Dystrophy, CHN, Cholestasis, Cholestasis Hereditary Norwegian Type, Cholestasis Intrahepatic, Cholestasis Neonatal, Cholestasis of Oral Contraceptive Users, Cholestasis with Peripheral Pulmonary Stenosis, Cholestasis of Pregnancy, Cholesterol Desmolase Deficiency, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Choreoacanthocytosis, Chorionic Villi Sampling, Chorioretinal Anomalies, Chorioretinal Anomalies with ACC, Chorireninal Coloboma-Joubert Syndrome, Choroidal Sclerosis, Choroideremia, Chotzen Syndrome, Christ-Siemens-Touraine Syndrome, Christ-Siemans-Touraine Syndrome, Christmas Disease, Christmas Tree Syndrome, Chromosome 3 Deletion of Distal 3p, Chromosome 3 Distal 3p Monosomy, Chromosome 3-Distal 3q2 Duplication, Chromosome 3-Distal 3q2 Trisomy, Chromosome 3 Monosomy 3p2, Chromosome 3q Partial Duplication Syndrome, Chromosome 3q, Partial Trisomy Syndrome, Chromosome 3-Trisomy 3q2, Chromosome 4 Deletion 4q31-qter Syndrome, Chromosome 4 Deletion 4q32-qter Syndrome, Chromosome 4 Deletion 4q33-qter Syndrome, Chromosome 4 Long Arm Deletion, Chromosome 4 Long Arm Deletion, Chromosome 4 Monosomy 4q, Chromosome 4-Monosomy 4q, Chromosome 4 Monosomy Distal 4q, Chromosome 4 Partial Deletion 4p, Chromosome 4, Partial Deletion of the Short Arm, Chromosome 4 Partial Monosomy of Distal 4q, Chromosome 4 Partial Monosomy 4p, Chromosome 4 Partial Trisomy 4 (q25-qter), Chromosome 4 Partial Trisomy 4 (q26 or q27-qter), Chromosome 4 Partial Trisomy 4 (q31 or 32-qter), Chromosome 4 Partial Trisomy 4p, Chromosome 4 Partial Trisomies 4q2 and 4q3, Chromosome 4 Partial Trisomy Distal 4, Chromosome 4 Ring, Chromosome 4 4q Terminal Deletion Syndrome, Chromosome 4q-Syndrome, Chromosome 4q-Syndrome, Chromosome 4 Trisomy 4, Chromosome 4 Trisomy 4p, Chromosome 4 XY/47 XXY (Mosiac), Chromosome 5 Monosomy 5p, Chromosome 5, Partial Deletion of the Short Arm Syndrome, Chromosome 5 Trisomy 5p, Chromosome 5 Trisomy 5p Complete (5p11-pter), Chromosome 5 Trisomy 5p Partial (5p13 or 14-pter), Chromosome 5p-Syndrome, Chromosome 6 Partial Trisomy 6q, Chromosome 6 Ring, Chromosome 6 Trisomy 6q2, Chromosome 7 Monosomy 7p2, Chromosome 7 Partial Deletion of Short Arm (7p2-), Chromosome 7 Terminal 7p Deletion [del (7) (p21-p22)], Chromosome 8 Monosomy 8p2, Chromosome 8 Monosomy 8p21-pter, Chromosome 8 Partial Deletion (short arm), Chromosome 8 Partial Monosomy 8p2, Chromosome 9 Complete Trisomy 9P, Chromosome 9 Partial Deletion of Short Arm, Chromosome 9 Partial Monosomy 9p, Chromosome 9 Partial Monosomy 9p22, Chromosome 9 Partial Monosomy 9p22-pter, Chromosome 9 Partial Trisomy 9P Included, Chromosome 9 Ring, Chromosome 9 Tetrasomy 9p, Chromosome 9 Tetrasomy 9p Mosaicism, Chromosome 9 Trisomy 9p (Multiple Variants), Chromosome 9 Trisomy 9 (pter-p21 to q32) Included, Chromosome 9 Trisomy Mosaic, Chromosome 9 Trisomy Mosaic, Chromosome 10 Distal Trisomy 10q, Chromosome 10 Monosomy, Chromosome 10 Monosomy 10p, Chromosome 10, Partial Deletion (short arm), Choromsome 10, 10p-Partial, Chromosome 10 Partial Trisomy 10q24-qter, Chromosome 10 Trisomy 10q2, Partial Monosomy of Long Arm of Chromosome 11, Chromosome 11 Partial Monosomy 11q, Chromosome 11 Partial Trisomy, Chromosome 11 Partial Trisomy 11q13-qter, Chromosome 11 Partial Trisomy 11q21-qter, Chromosome 11 Partial Trisomy 11q23-qter, Chromosome 11q,Partial Trisomy, Chromosome 12 Isochromosome 12p Mosaic, Chromosome 13 Partial Monosomy 13q, Chromosome 13, Partial Monosomy of the Long Arm, Chromosome 14 Ring, Chromosome 14 Trisomy, Chromosome 15 Distal Trisomy 15q, Chromosome r15, Chromosome 15 Ring, Chromosome 15 Trisomy 15q2, Chromosome 15q, Partial Duplication Syndrome, Chromosome 17 Interstitial Deletion 17p, Chromosome 18 Long Arm Deletion Syndrome, Chromosome 18 Monosomy 18p, Chromosome 18 Monosomy 18Q, Chromosome 18 Ring, Chromosome 18 Tetrasomy 18p, Chromosome 18q-Syndrome, Chromosome 21 Mosaic 21 Syndrome, Chromosome 21 Ring, Chromosome 21 Translocation 21 Syndrome, Chromosome 22 Inverted Duplication (22pter-22q11), Chromosome 22 Partial Trisomy (22pter-22q11), Chromosome 22 Ring, Chromosome 22 Trisomy Mosaic, Chromosome 48 XXYY, Chromosome 48 XXXY, Chromosome r15, Chromosomal Triplication, Chromosome Triplication, Chromosome Triploidy Syndrome, Chromosome X, Chromosome XXY, Chronic Acholuric Jaundice, Chronic Adhesive Arachnoiditis, Chronic Adrenocortical Insufficiency, Chronic Cavernositis, Chronic Congenital Aregenerative Anemia, Chronic Dysphagocytosis, Chronic Familial Granulomatosis, Chronic Familial Icterus, Chronic Fatigue Immune Dysfunction Syndrome (CFIDS), Chronic Granulomatous Disease, Chronic Guillain-Barre Syndrome, Chronic Idiopathic Jaundice, Chronic Idiopathic Polyneuritis (CIP), Chronic Inflammatory Demyelinating Polyneuropathy, Chronic Inflammatory Demyelinating Polyradiculoneuropathy, Chronic Motor Tic, Chronic Mucocutaneous Candidiasis, Chronic Multiple Tics, Chronic Non-Specific Ulcerative Colitis, Chronic Obliterative Cholangitis, Chronic Peptic Ulcer and Esophagitis Syndrome, Chronic Progressive Chorea, Chronic Progressive External Ophthalmoplegia Syndrome, Chronic Progressive External Ophthalmoplegia and myopathy, Chronic Progressive External Ophthalmoplegia with Ragged Red Fibers, Chronic Relapsing Polyneuropathy, Chronic Sarcoidosis, Chronic Spasmodic Dysphonia, Chronic Vomiting in Childhood, CHS, Churg-Strauss Syndrome, Cicatricial Pemphigoid, CIP, Cirrhosis Congenital Pigmentary, Cirrhosis, Cistinuria, Citrullinemia, CJD, Classic Schindler Disease, Classic Type Pfeiffer Syndrome, Classical Maple Syrup Urine Disease, Classical Hemophilia, Classical Form Cockayne Syndrome Type I (Type A), Classical Leigh's Disease, Classical Phenylketonuria, Classical X-Linked Pelizaeus-Merzbacher Brain Sclerosis, CLE, Cleft Lip/Palate Mucous Cysts Lower Lip PP Digital and Genital Anomalies, Cleft Lip-Palate Blepharophimosis Lagophthalmos and Hypertelorism, Cleft Lip/Palate with Abnormal Thumbs and Microcephaly, Cleft palate-joint contractures-dandy walker malformations, Cleft Palate and Cleft Lip, Cleidocranial Dysplasia w/Micrognathia, Absent Thumbs, & Distal Aphalangia, Cleidocranial Dysostosis, Cleidocranial Dysplasia, Click murmur syndrome, CLN1, Clonic Spasmodic, Cloustons Syndrome, Clubfoot, CMDI, CMM, CMT, CMTC, CMTX, COA Syndrome, Coarctation of the aorta, Coats' Disease, Cobblestone dysplasia, Cochin Jewish Disorder, Cockayne Syndrome, COD-MD Syndrome, COD, Coffin Lowry Syndrome, Coffin Syndrome, Coffin Siris Syndrome, COFS Syndrome, Cogan Corneal Dystrophy, Cogan Reese Syndrome, Cohen Syndrome, Cold Agglutinin Disease, Cold Antibody Disease, Cold Antibody Hemolytic Anemia, Colitis Ulcerative, Colitis Gravis, Colitis Ulcerative Chronic Non-Specific Ulcerative Colitis, Collodion Baby, Coloboma Heart Defects Atresia of the Choanae Retardation of Growth and Development Genital and Urinary Anomalies and Ear Anomalies, Coloboma, Colonic Neurosis, Color blindness, Colour blindness, Colpocephaly, Columnar-Like Esophagus, Combined Cone-Rod Degeneration, Combined Immunodeficiency with Immunoglobulins, Combined Mesoectodermal Dysplasia, Common Variable Hypogammaglobulinemia, Common Variable Immunodeficiency, Common Ventricle, Communicating Hydrocephalus, Complete Absense of Hypoxanthine-Guanine Phosphoribosyltranferase, Complete Atrioventricular Septal Defect, Complement Component 1 Inhibitor Deficiciency, Complement Component C1 Regulatory Component Deficiency, Complete Heart Block, Complex Carbohydrate Intolerance, Complex Regional Pain Syndrome, Complex V ATP Synthase Deficiency, Complex I, Complex I NADH dehydrogenase deficiency, Complex II, Complex II Succinate dehydrogenase deficiency, Complex III, Complex III Ubiquinone-cytochrome c oxidoreductase deficiency, Complex IV, Complex IV Cytochrome c oxidase deficiency, Complex IV Deficiency, Complex V, Cone-Rod Degeneration, Cone-Rod Degeneration Progressive, Cone Dystrophy, Cone-Rod Dystrophy, Confluent Reticular Papillomatosis, Congenital with low PK Kinetics, Congenital Absence of Abdominal Muscles, Congenital Absence of the Thymus and Parathyroids, Congenital Achromia, Congenital Addison's Disease, Congenital Adrenal Hyperplasia, Congenital Adreneal Hyperplasia, Congenital Afibrinogenemia, Congenital Alveolar Hypoventilation, Congenital Anemia of Newborn, Congenital Bilateral Persylvian Syndrome, Congenital Brown Syndrome, Congenital Cardiovascular Defects, Congenital Central Hypoventilation Syndrome, Congenital Cerebral Palsy, Congenital Cervical Synostosis, Congenital Clasped Thunb with Mental Retardation, Congenital Contractural Arachnodactyly, Congenital Contractures Multiple with Arachnodactyly, Congenital Cyanosis, Congenital Defect of the Skull and Scalp, Congenital Dilatation of Intrahepatic Bile Duct, Congenital Dysmyelinating Neuropathy, Congenital Dysphagocytosis, Congenital Dysplastic Angiectasia, Congenital Erythropoietic Porphyria, Congenital Factor XIII Deficiency, Congenital Failure of Autonomic Control of Respiration, Congenital Familial Nonhemolytic Jaundice Type I, Congenital Familial Protracted Diarrhea, Congenital Form Cockayne Syndrome Type II (Type B), Congenital Generalized Fibromatosis, Congenital German Measles, Congenital Giant Axonal Neuropathy, Congenital Heart Block, Congenital Heart Defects, Congenital Hemidysplasia with Ichthyosis Erythroderma and Limb Defects, Congenital Hemolytic Jaundice, Congenital Hemolytic Anemia, Congenital Hepatic Fibrosis, Congenital Hereditary Corneal Dystrophy, Congenital Hereditary Lymphedema, Congenital Hyperchondroplasia, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erytiroderma, Congenital Keratoconus, Congenital Lactic Acidosis, Congenital Lactose Intolerance, Congenital Lipodystrophy, Congenital Liver Cirrhosis, Congenital Lobar Emphysema, Congenital Localized Emphysema, Congenital Macroglossia, Congenital Medullary Stenosis, Congenital Megacolon, Congenital Melanocytic Nevus, Congenital Mesodermal Dysmorphodystrophy, Congenital Mesodermal Dystrophy, Congenital Microvillus Atrophy, Congenital Multiple Arthrogryposis, Congenital Myotonic Dystrophy, Congenital Neuropathy caused by Hypomyelination, Congenital Pancytopenia, Congenital Pernicious Anemia, Congenital Pernicious Anemia due to Defect of Intrinsic Factor, Congenital Pernicious Anemia due to Defect of Intrinsic Factor, Congenital Pigmentary Cirrhosis, Congenital Porphyria, Congenital Proximal myopathy Associated with Desmin Storage myopathy, Congenital Pulmonary Emphysema, Congenital Pure Red Cell Anemia, Congenital Pure Red Cell Aplasia, Congenital Retinal Blindness, Congenital Retinal Cyst, Congenital Retinitis Pigmentosa, Congenital Retinoschisis, Congenital Rod Disease, Congenital Rubella Syndrome, Congenital Scalp Defects with Distal Limb Reduction Anomalies, Congenital Sensory Neuropathy, Congenital SMA with arthrogryposis, Congenital Spherocytic Anemia, Congenital Spondyloepiphyseal Dysplasia, Congenital Tethered Cervical Spinal Cord Syndrome, Congenital Tyrosinosis, Congenital Varicella Syndrome, Congenital Vascular Cavernous Malformations, Congenital Vascular Veils in the Retina, Congenital Word Blindness, Congenital Wandering Spleen (Pediatric), Congestive Cardio myopathy, Conical Cornea, Conjugated Hyperbilirubinemia, Conjunctivitis, Conjunctivitis Ligneous, Conjunctivo-Urethro-Synovial Syndrome, Conn's Syndrome, Connective Tissue Disease, Conradi Disease, Conradi Hunermann Syndrome, Constitutional Aplastic Anemia, Constitutional Erythroid Hypoplasia, Constitutional Eczema, Constitutional Liver Dysfunction, Constitutional Thrombopathy, Constricting Bands Congenital, Constrictive Pericarditis with Dwarfism, Continuous Muscle Fiber Activity Syndrome, Contractural Arachnodactyly, Contractures of Feet Muscle Atrophy and Oculomotor Apraxia, Convulsions, Cooley's anemia, Copper Transport Disease, Coproporphyria Porphyria Hepatica, Cor Triatriatum, Cor Triatriatum Sinistrum, Cor Triloculare Biatriatum, Cor Biloculare, Cori Disease, Cornea Dystrophy, Corneal Amyloidosis, Corneal Clouding-Cutis Laxa-Mental Retardation, Corneal Dystrophy, Cornelia de Lange Syndrome, Coronal Dentine Dysplasia, Coronary Artery Disease, Coronary Heart Disease, Corpus Callosum Agenesis, Cortical-Basal Ganglionic Degeneration, Corticalis Deformaris, Cortico-Basal Ganglionic Degeneration (CBGD), Corticobasal Degeneration, Corticosterone Methloxidase Deficiency Type I, Corticosterone Methyloxidase Deficiency Type II, Cortisol, Costello Syndrome, Cot Death, COVESDEM Syndrome, COX, COX Deficiency, COX Deficiency French-Canadian Type, COX Deficiency Infantile Mitochondrial myopathy de Toni-Fanconi-Debre included, COX Deficiency Type Benign Infantile Mitochondrial Myopathy, CP, CPEO, CPEO with myopathy, CPEO with Ragged-Red Fibers, CPPD Familial Form, CPT Deficiency, CPTD, Cranial Arteritis, Cranial Meningoencephalocele, Cranio-Oro-Digital Syndrome, Craniocarpotarsal dystrophy, Craniocele, Craniodigital Syndrome-Mental Retardation Scott Type, Craniofacial Dysostosis, Craniofacial Dysostosis-PD Arteriosus-Hypertrichosis-Hypoplasia of Labia, Craniofrontonasal Dysplasia, Craniometaphyseal Dysplasia, Cranioorodigital Syndrome, Cranioorodigital Syndrome Type II, Craniostenosis Crouzon Type, Craniostenosis, Craniosynostosis-Choanal Atresia-Radial Humeral Synostosis, Craniosynostosis-Hypertrichosis-Facial and Other Anomalies, Craniosynostosis Midfacial Hypoplasia and Foot Abnormalities, Craniosynostosis Primary, Craniosynostosis-Radial Aplasia Syndrome, Craniosynostosis with Radial Defects, Cranium Bifidum, CREST Syndrome, Creutzfeldt Jakob Disease, Cri du Chat Syndrome, Crib Death, Crigler Najjar Syndrome Type I, Crohn's Disease, Cronkhite-Canada Syndrome, Cross Syndrome, Cross' Syndrome, Cross-McKusick-Breen Syndrome, Crouzon, Crouzon Syndrome, Crouzon Craniofacial Dysostosis, Cryoglobulinemia Essential Mixed, Cryptophthalmos-Syndactyly Syndrome, Cryptorchidism-Dwarfism-Subnormal Mentality, Crystalline Corneal Dystrophy of Schnyder, CS, CSD, CSID, CSO, CST Syndrome, Curly Hair-Ankyloblephanon-Nail Dysplasia, Curschmann-Batten-Steinert Syndrome, Curth Macklin Type Ichthyosis Hystric, Curti-Macklin Type, Cushing's, Cushing Syndrome, Cushing's m, Cutaneous Malignant Melanoma Hereditary, Cutaneous Porphyrias, Cutis Laxa, Cutis Laxa-Growth Deficiency Syndrome, Cutis Marmorata Telangiectatica Congenita, CVI, CVID, CVS, Cyclic vomiting syndrome, Cystic Disease of the Renal Medulla, Cystic Hygroma, Cystic Fibrosis, Cystic Lymphangioma, Cystine-Lysine-Arginine-Ornithinuria, Cystine Storage Disease, Cystinosis, Cystinuria, Cystinuria with Dibasic Aminoaciduria, Cystinuria Type I, Cystinuria Type II, Cystinuria Type III, Cysts of the Renal Medulla Congenital, Cytochrome C Oxidase Deficiency, D.C., Dacryosialoadenopathy, Dacryosialoadenopathia, Dalpro, Dalton, Daltonism, Danbolt-Cross Syndrome, Dancing Eyes-Dancing Feet Syndrome, Dandy-Walker Syndrome, Dandy-Walker Cyst, Dandy-Walker Deformity, Dandy Walker Malformation, Danish Cardiac Type Amyloidosis (Type III), Darier Disease, Davidson's Disease, Davies' Disease, DBA, DBS, DC, DD, De Barsy Syndrome, De Barsy-Moens-Diercks Syndrome, de Lange Syndrome, De Morsier Syndrome, De Santis Cacchione Syndrome, de Toni-Fanconi Syndrome, Deafness Congenital and Functional Heart Disease, Deafness-Dwarfism-Retinal Atrophy, Deafness-Functional Heart Disease, Deafness Onychodystrophy Osteodystrophy and Mental Retardation, Deafness and Pili Torti Bjornstad Type, Deafness Sensorineural with Imperforate Anus and Hypoplastic Thumbs, Debrancher Deficiency, Deciduous Skin, Defect of Enterocyte Intrinsic Factor Receptor, Defect in Natural Killer Lymphocytes, Defect of Renal Reabsorption of Carnitine, Deficiency of Glycoprotein Neuraminidase, Deficiency of Mitochondrial Respiratory Chain Complex IV, Deficiency of Platelet Glycoprotein Ib, Deficiency of Von Willebrand Factor Receptor, Deficiency of Short-Chain Acyl-CoA Dehydrogenase (ACADS), Deformity with Mesomelic Dwarfism, Degenerative Chorea, Degenerative Lumbar Spinal Stenosis, Degos Disease, Degos-Kohlmeier Disease, Degos Syndrome, DEH, Dejerine-Roussy Syndrome, Dejerine Sottas Disease, Deletion 9p Syndrome Partial, Deletion 11q Syndrome Partial, Deletion 13q Syndrome Partial, Delleman-Oorthuys Syndrome, Delleman Syndrome, Dementia with Lobar Atrophy and Neuronal Cytoplasmic Inclusions, Demyelinating Disease, DeMyer Syndrome, Dentin Dysplasia Coronal, Dentin Dysplasia Radicular, Dentin Dysplasia Type I, Dentin Dysplasia Type II, Dentinogenesis Imperfecta Brandywine type, Dentinogenesis Imperfecta Shields Type, Dentinogenesis Imperfecta Type III, Dento-Oculo-Osseous Dysplasia, Dentooculocutaneous Syndrome, IDenys-Drash Syndrome, Depakene, DepakeneTM exposure, Depakote, Depakote Sprinkle, Depigmentation-Gingival Fibromatosis-Microphthalmia, Dercum Disease, Dermatitis Atopic, Dermatitis Exfoliativa, Dermatitis Herpetiformis, Dermatitis Multiformis, Dermatochalasia Generalized, Dermatolysis Generalized, Dermatomegaly, Dermatomyositis sine myositis, Dermatomyositis, Derrnatosparaxis, Dermatostomatitis Stevens Johnson Type, Desbuquois Syndrome, Desmin Storage myopathy, Desquamation of Newborn, Deuteranomaly, Developmental Reading Disorder, Developmental Gerstmann Syndrome, Devergie Disease, Devic Disease, Devic Syndrome, Dextrocardia-Bronchiectasis and Sinusitis, Dextrocardia with Situs Inversus, DGS, DGSX Golabi-Rosen Syndrome Included, DH, DHAP alkyl transferase deficiency, DHBS Deficiency, DHOF, DHPR Deficiency, Diabetes Insipidus, Diabetes Insipidus Diabetes Mellitus Optic Atrophy and Deafness, Diabetes Insipidus Neurohypophyseal, Diabetes Insulin Dependent, Diabetes Mellitus, Diabetes Mellitus Addison's Disease Myxedema, Diabetic Acidosis, Diabetic Bearded Woman Syndrome, Diamond-Blackfan Anemia, Diaphragmatic Apnea, Diaphyseal Aclasis, Diastrophic Dwarfism, Diastrophic Dysplasia, Diastrophic Nanism Syndrome, Dicarboxylic Aminoaciduria, Dicarboxylicaciduria Caused by Defect in Beta-Oxidation of Fatty Acids, Dicarboxylicaciduria due to Defect in Beta-Oxidation of Fatty Acids, Dicarboxylicaciduria due to MCADH Deficiency, Dichromasy, Dicker-Opitz, DIDMOAD, Diencephalic Syndrome, Diencephalic Syndrome of Childhood, Diencephalic Syndrome of Emaciation, Dienoyl-CoA Reductase Deficiency, Diffuse Cerebral Degeneration in Infancy, Diffuse Degenerative Cerebral Disease, Diffuse Idiopathic Skeletal Hyperostosis, Diffusum-Glycopeptiduria, DiGeorge Syndrome, Digital-Oro-Cranio Syndrome, Digito-Oto-Palatal Syndrome, Digito-Oto-Palatal Syndrome Type I, Digito-Oto-Palatal Syndrome Type II, Dihydrobiopterin Synthetase Deficiency, Dihydropteridine Reductase Deficiency, Dihydroxyacetonephosphate synthase, Dilated (Congestive) Cardio myopathy, Dimitri Disease, Diplegia of Cerebral Palsy, Diplo-Y Syndrome, Disaccharidase Deficiency, Disaccharide Intolerance I, Discoid Lupus, Discoid Lupus Erythematosus, DISH, Disorder of Cornification, Disorder of Cornification Type I, Disorder of Cornification 4, Disorder of Cornification 6, Disorder of Cornification 8, Disorder of Cornification 9 Netherton's Type, Disorder of Cornification 11 Phytanic Acid Type, Disorder of Cornification 12 Neutral Lipid Storage Type), Disorder of Conification 13, Disorder of Cornification 14, Disorder of Cornification 14 Trichothiodystrophy Type, Disorder of Cornification 15 (Keratitis Deafness Type), Disorder of Cornification 16, Disorder of Cornification 18 Erythrokeratodermia Variabilis Type, Disorder of Cornification 19, Disorder of Cornification 20, Disorder of Cornification 24, Displaced Spleen, Disseminated Lupus Erythematosus, Disseminated Neurodermatitis, Disseminated Sclerosis, Distal 11q Monosomy, Distal 11q-Syndrome, Distal Arthrogryposis Multiplex Congenita Type IIA, Distal Arthrogryposis Multiplex Congenita Type IIA, Distal Arthrogryposis Type IIA, Distal Arthrogryposis Type 2A, Distal Duplication 6q, Distal Duplication 10q, Dup(10q) Syndrome, Distal Duplication 15q, Distal Monosomy 9p, Distal Trisomy 6q, Distal Trisomy 10q Syndrome, Distal Trisomy 11q, Divalproex, DJS, DKC, DLE, DLPIII, DM, DMC Syndrome, DMC Disease, DMD, DNS Hereditary, DOC I, DOC 2, DOC 4, DOC 6 (Harlequin Type), DOC 8 Curth-Macklin Type, DOC 11 Phytanic Acid Type, DOC 12 (Neutral Lipid Storage Type), DOC 13, DOC 14, DOC 14 Trichothiodystrophy Type, DOC 15 (Keratitis Deafness Type), DOC 16, DOC 16 Unilateral Hemidysplasia Type, DOC 18, DOC 19, DOC 20, DOC 24, Dohle's Bodies-Myelopathy, Dolichospondylic Dysplasia, Dolichostenomelia, Dolichostenomelia Syndrome, Dominant Type Kenny-Caffe Syndrome, Dominant Type Myotonia Congenita, Donahue Syndrome, Donath-Landsteiner Hemolytic Anemia, Donath-Landsteiner Syndrome, DOOR Syndrome, DOORS Syndrome, Dopa-responsive Dystonia (DRD), Dorfman Chanarin Syndrome, Dowling-Meara Syndrome, Down Syndrome, DR Syndrome, Drash Syndrome, DRD, Dreifuss-Emery Type Muscular Dystrophy with Contractures, Dressler Syndrome, Drifting Spleen, Drug-induced Acanthosis Nigricans, Drug-induced Lupus Erythematosus, Drug-related Adrenal Insufficiency, Drummond's Syndrome, Dry Beriberi, Dry Eye, DTD, Duane's Retraction Syndrome, Duane Syndrome, Duane Syndrome Type IA 1B and 1C, Duane Syndrome Type 2A 2B and 2C, Duane Syndrome Type 3A 3B and 3C, Dubin Johnson Syndrome, Dubowitz Syndrome, Duchenne, Duchenne Muscular Dystrophy, Duchenne's Paralysis, Duhring's Disease, Duncan Disease, Duncan's Disease, Duodenal Atresia, Duodenal Stenosis, Duodenitis, Duplication 4p Syndrome, Duplication 6q Partial, Dupuy's Syndrome, Dupuytren's Contracture, Dutch-Kennedy Syndrome, Dwarfism, Dwarfism Campomelic, Dwarfism Cortical Thickening of the Tubular Bones & Transient Hypocalcemia, Dwarfism Levi's Type, Dwarfism Metatropic, Dwarfism-Onychodysplasia, Dwarfism-Pericarditis, Dwarfism with Renal Atrophy and Deafness, Dwarfism with Rickets, DWM, Dyggve Melchior Clausen Syndrome, Dysautonomia Familial, Dysbetalipoproteinemia Familial, Dyschondrodysplasia with Hemangiomas, Dyschondrosteosis, Dyschromatosis Universalis Hereditaria, Dysencephalia Splanchnocystica, Dyskeratosis Congenita, Dyskeratosis Congenita Autosomal Recessive, Dyskeratosis Congenita Scoggins Type, Dyskeratosis Congenita Syndrome, Dyskeratosis Follicularis Vegetans, Dyslexia, Dysmyelogenic Leukodystrophy, Dysmyelogenic Leukodystrophy-Megalobare, Dysphonia Spastica, Dysplasia Epiphysialis Punctata, Dysplasia Epiphyseal Hemimelica, Dysplasia of Nails With Hypodontia, Dysplasia Cleidocranial, Dysplasia Fibrous, Dysplasia Gigantism SyndromeX-Linked, Dysplasia Osteodental, Dysplastic Nevus Syndrome, Dysplastic Nevus Type, Dyssynergia Cerebellaris Myoclonica, Dyssynergia Esophagus, Dystonia, Dystopia Canthorum, Dystrophia Adiposogenitalis, Dystrophia Endothelialis Cornea, Dystrophia Mesodermalis, Dystrophic Epidermolysis Bullosa, Dystrophy, Asphyxiating Thoracic, Dystrophy Myotonic, E-D Syndrome, Eagle-Barrett Syndrome, Eales Retinopathy, Eales Disease, Ear Anomalies-Contractures-Dysplasia of Bone with Kyphoscoliosis, Ear Patella Short Stature Syndrome, Early Constraint Defects, Early Hypercalcemia Syndrome with Elfin Facie, Early-onset Dystonia, Eaton Lambert Syndrome, EB, Ebstein's anomaly, EBV Susceptibility (EBVS), EBVS, ECD, ECPSG, Ectodermal Dysplasias, Ectodermal Dysplasia Anhidrotic with Cleft Lip and Cleft Palate, Ectodermal Dysplasia-Exocrine Pancreatic Insufficiency, Ectodermal Dysplasia Rapp-Hodgkin type, Ectodermal and Mesodermal Dysplasia Congenital, Ectodermal and Mesodermal Dysplasia with Osseous Involvement, Ectodermosis Erosiva Pluriorificialis, Ectopia Lentis, Ectopia Vesicae, Ectopic ACTH Syndrome, Ectopic Adrenocorticotropic Hormone Syndrome, Ectopic Anus, Ectrodactilia of the Hand, Ectrodactyly, Ectrodactyly-Ectodermal Dysplasia-Clefting Syndrome, Ectrodactyly Ectodermal Dysplasias Clefting Syndrome, Ectrodactyly Ectodermal Dysplasia Cleft Lip/Cleft Palate, Eczema, Eczema-Thrombocytopenia-Immunodeficiency Syndrome, EDA, EDMD, EDS, EDS Arterial-Ecchymotic Type, EDS Arthrochalasia, EDS Classic Severe Form, EDS Dysfibronectinemic, EDS Gravis Type, EDS Hypermobility, EDS Kyphoscoliotic, EDS Kyphoscoliosis, EDS Mitis Type, EDS Ocular-Scoliotic, EDS Progeroid, EDS Periodontosis, EDS Vascular, EEC Syndrome, EFE, EHBA, EHK, Ehlers Danlos Syndrome, Ehlers-Danlos syndrome, Ehlers Danlos IX, Eisenmenger Complex, Eisenmenger's complex, Eisemnenger Disease, Eisenmenger Reaction, Eisenmenger Syndrome, Ekbom Syndrome, Ekman-Lobstein Disease, Ektrodactyly of the Hand, EKV, Elastin fiber disorders, Elastorrhexis Generalized, Elastosis Dystrophica Syndrome, Elective Mutism (obsolete), Elective Mutism, Electrocardiogram (ECG or EKG), Electron Transfer Flavoprotein (ETF) Dehydrogenase Deficiency: (GAII & MADD), Electrophysiologic study (EPS), Elephant Nails From Birth, Elephantiasis Congenita Angiomatosa, Hemangiectatic Hypertrophy, Elfin Facies with Hypercalcemia, Ellis-van Creveld Syndrome, Ellis Van Creveld Syndrome, Embryoma Kidney, Embryonal Adenomyosarcoma Kidney, Embryonal Carcinosarcoma Kidney, Embryonal Mixed Tumor Kidney, EMC, Emery Dreyfus Muscular Dystrophy, Emery-Dreifuss Muscular Dystrophy, Emery-Dreifuss Syndrome, EMF, EMG Syndrome, Empty Sella Syndrome, Encephalitis Periaxialis Difflusa, Encephalitis Periaxialis Concentrica, Encephalocele, Encephalofacial Angiomatosis, Encephalopathy, Encephalotrigeminal Angiomatosis, Enchondromatosis with Multiple Cavernous Hemangiomas, Endemic Polyneuritis, Endocardial Cushion Defect, Endocardial Cushion Defects, Endocardial Dysplasia, Endocardial Fibroelastosis (EFE), Endogenous Hypertriglyceridemia, Endolymphatic Hydrops, Endometrial Growths, Endometriosis, Endomyocardial Fibrosis, Endothelial Corneal Dystrophy Congenital, Endothelial Epithelial Corneal Dystrophy, Endothelium, Engelmann Disease, Enlarged Tongue, Enterocolitis, Enterocyte Cobalamin Malabsorption, Eosinophia Syndrome, Eosinophilic Cellulitis, Eosinophilic Fasciitis, Eosinophilic Granuloma, Eosinophilic Syndrome, Epidermal Nevus Syndrome, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Epilepsia Procursiva, Epilepsy, Epinephrine, Epiphyseal Changes and High Myopia, Epiphyseal Osteochondroma Benign, Epiphysealis Hemimelica Dysplasia, Episodic-Abnormal Eye Movement, Epithelial Basement Membrane Corneal Dystrophy, Epithelial Corneal Dystrophy of Meesmann Juvenile, Epitheliomatosis Multiplex with Nevus, Epithelium, Epival, EPS, Epstein-Barr Virus-Induced Lymphoproliferative Disease in Males, Erb-Goldflam syndrome, Erdheim Chester Disease, Erythema Multiforme Exudativum; Erythema Polymorphe Stevens Johnson Type, Erythroblastophthisis, Erythroblastosis Fetalis, Erythroblastosis Neonatorum, Erythroblastotic Anemia of Childhood, Erythrocyte Phosphoglycerate Kinase Deficiency, Erythrogenesis Imperfecta, Erythrokeratodermia Progressiva Symmetrica, Erythrokeratodermia Progressiva Symmetrica Ichthyosis, Erythrokeratodermia Variabilis, Erythrokeratodermia Variabilis Type, Erythrokeratolysis Hiemalis, Erythropoietic Porphyrias, Erythropoietic Porphyria, Escobar Syndrome, Esophageal Atresia, Esophageal Aperistalsis, Esophagitis-Peptic Ulcer, Esophagus Atresia and/or Tracheoesophageal Fistula, Essential Familial Hyperlipemia, Essential Fructosuria, Essential Hematuria, Essential Hemorrhagic Thrombocythemia, Essential Mixed Cryoglobulinemia, Essential Moschowitz Disease, Essential Thrombocythemia, Essential Thrombocytopenia, Essential Thrombocytosis, Essential Tremor, Esterase Inhibitor Deficiency, Estren-Dameshek variant of Fanconi Anemia, Estrogen-related Cholestasis, ET, ETF, Ethylmalonic Adipicaciduria, Eulenburg Disease, pc, EVCS, Exaggerated Startle Reaction, Exencephaly, Exogenous Hypertriglyceridemia, Exomphalos-Macroglossia-Gigantism Syndrom, Exophthalmic Goiter, Expanded Rubella Syndrome, Exstrophy of the Bladder, EXT, External Chondromatosis Syndrome, Extrahepatic Biliary Atresia, Extramedullary Plasmacytoma, Exudative Retinitis, Eye Retraction Syndrome, FA1, FAA, Fabry Disease, FAC, FACB, FACD, FACE, FACF, FACG, FACH, Facial Nerve Palsy, Facial Paralysis, Facial Ectodermal Dysplasias, Facial Ectodermal Dysplasia, Facio-Scapulo-Humeral Dystrophy, Facio-Auriculo-Vertebral Spectrum, Facio-cardio-cutaneous syndrome, Facio-Fronto-Nasal Dysplasia, Faciocutaneoskeletal Syndrome, Faciodigitogenital syndrome, Faciogenital dysplasia, Faciogenitopopliteal Syndrome, Faciopalatoosseous Syndrome, Faciopalatoosseous Syndrome Type II, Facioscapulohumeral muscular dystrophy, Factitious Hypoglycemia, Factor VIII Deficiency, Factor IX Deficiency, Factor XI Deficiency, Factor XII deficiency, Factor XIII Deficiency, Fahr Disease, Fahr's Disease, Failure of Secretion Gastric Intrinsic Factor, Fairbank Disease, Fallot's Tetralogy, Familial Acrogeria, Familial Acromicria, Familial Adenomatous Colon Polyposis, Familial Adenomatous Polyposis with Extraintestinal Manifestations, Familial Alobar Holoprosencephaly, Familial Alpha-Lipoprotein Deficiency, Familial Amyotrophic Chorea with Acanthocytosis, Familial Arrhythmic Myoclonus, Familial Articular Chondrocalcinosis, Familial Atypical Mole-Malignant Melanoma Syndrome, Familial Broad Beta Disease, Familial Calcium Gout, Familial Calcium Pyrophosphate Arthropathy, Familial Chronic Obstructive Lung Disease, Familial Continuous Skin Peeling, Familial Cutaneous Amyloidosis, Familial Dysproteinemia, Familial Emphysema, Familial Enteropathy Microvillus, Familial Foveal Retinoschisis, Familial Hibernation Syndrome, Familial High Cholesterol, Familial Hemochromatosis, Familial High Blood Cholesterol, Familial High-Density Lipoprotein Deficiency, Familial High Serum Cholesterol, Familial Hyperlipidema, Familial Hypoproteinemia with Lymphangietatic Enteropathy, Familial Jaundice, Familial Juvenile Nephronophtisis-Associated Ocular Anomaly, Familial Lichen Amyloidosis (Type IX), Familial Lumbar Stenosis, Familial Lymphedema Praecox, Familial Mediterranean Fever, Familial Multiple Polyposis, Familial Nuchal Bleb, Familial Paroxysmal Polyserositis, Familial Polyposis Coli, Familial Primary Pulmonary Hypertension, Familial Renal Glycosuria, Familial Splenic Anemia, Familial Startle Disease, Familial Visceral Amyloidosis (Type VIII), FAMMM, FANCA, FANCB, FANCC, FANCD, FANCE, Fanconi Panmyelopathy, Fanconi Pancytopenia, Fanconi II, Fanconi's Anemia, Fanconi's Anemia Type I, Fanconi's Anemia Complementation Group, Fanconi's Anemia Complementation Group A, Fanconi's Anemia Complementation Group B, Fanconi's Anemia Complementation Group C, Fanconi's Anemia Complementation Group D, Fanconi's Anemia Complementation Group E, Fanconi's Anemia Complementation Group G, Fanconi's Anemia Complementation Group H, Fanconi's Anemia Estren-Dameshek Variant, FANF, FANG, FANH, FAP, FAPG, Farber's Disease, Farber's Lipogranulomatosis, FAS, Fasting Hypoglycemia, Fat-Induced Hyperlipemia, Fatal Granulomatous Disease of Childhood, Fatty Oxidation Disorders, Fatty Liver with Encephalopathy, FAV, FCH, FCMD, FCS Syndrome, FD, FDH, Febrile Mucocutaneous Syndrome Stevens Johnson Type, Febrile Neutrophilic Dermatosis Acute, Febrile Seizures, Feinberg's syndrome, Feissinger-Leroy-Reiter Syndrome, Female Pseudo-Turner Syndrome, Femoral Dysgenesis Bilateral-Robin Anomaly, Femoral Dysgenesis Bilateral, Femoral Facial Syndrome, Femoral Hypoplasia-Unusual Facies Syndrome, Fetal Alcohol Syndrome, Fetal Anti-Convulsant Syndrome, Fetal Cystic Hygroma, Fetal Effects of Alcohol, Fetal Effects of Chickenpox, Fetal Effects of Thalidomide, Fetal Effects of Varicella Zoster Virus, Fetal Endomyocardial Fibrosis, Fetal Face Syndrome, Fetal Iritis Syndrome, Fetal Transfusion Syndrome, Fetal Valproate Syndrome, Fetal Valproic Acid Exposure Syndrome, Fetal Varicefla Infection, Fetal Varicella Zoster Syndrome, FFDD Type II, FG Syndrome, FGDY, FHS, Fibrin Stabilizing Factor Deficiency, Fibrinase Deficiency, Fibrinoid Degeneration of Astrocytes, Fibrinoid Leukodystrophy, Fibrinoligase Deficiency, Fibroblastoma Perineural, Fibrocystic Disease of Pancreas, Fibrodysplasia Ossificans Progressiva, Fibroelastic Endocarditis, Fibromyalgia, Fibromyalgia-Fibromyositis, Fibromyositis, Fibrosing Cholangitis, Fibrositis, Fibrous Ankylosis of Multiple Joints, Fibrous Cavernositis, Fibrous Dysplasia, Fibrous Plaques of the Penis, Fibrous Sclerosis of the Penis, Fickler-Winkler Type, Fiedler Disease, Fifth Digit Syndrome, Filippi Syndrome, Finnish Type Amyloidosis (Type V), First Degree Congenital Heart Block, First and Second Branchial Arch Syndrome, Fischer's Syndrome, Fish Odor Syndrome, Fissured Tongue, Flat Adenoma Syndrome, Flatau-Schilder Disease, Flavin Containing Monooxygenase 2, Floating Beta Disease, Floating-Harbor Syndrome, Floating Spleen, Floppy Infant Syndrome, Floppy Valve Syndrome, Fluent aphasia, FMD, FMF, FMO Adult Liver Form, FMO2, FND, Focal Dermal Dysplasia Syndrome, Focal Dermal Hypoplasia, Focal Dermato-Phalangeal Dysplasia, Focal Dystonia, Focal Epilepsy, Focal Facial Dermal Dysplasia Type II, Focal Neuromyotonia, FODH, Folling Syndrome, Fong Disease, FOP, Forbes Disease, Forbes-Albright Syndrome, Forestier's Disease, Forsius-Eriksson Syndrome (X-Linked), Fothergill Disease, Fountain Syndrome, Foveal Dystrophy Progressive, FPO Syndrome Type II, FPO, Fraccaro Type Achondrogenesis (Type IB), Fragile X syndrome, Franceschetti-Zwalen-Klein Syndrome, Francois Dyscephaly Syndrome, Francois-Neetens Speckled Dystrophy, Flecked Comeal Dystrophy, Fraser Syndrome, FRAXA, FRDA, Fredrickson Type I Hyperlipoproteinemia, Freeman-Sheldon Syndrome, Freire-Maia Syndrome, Frey's Syndrome, Friedreich's Ataxia, Friedreich's Disease, Friedreich's Tabes, FRNS, Froelich's Syndrome, Frommel-Chiari Syndrome, Frommel-Chiari Syndrome Lactation-Uterus Atrophy, Frontodigital Syndrome, Frontofacionasal Dysostosis, Frontofacionasal Dysplasia, Frontonasal Dysplasia, Frontonasal Dysplasia with Coronal Craniosynostosis, Fructose-1-Phosphate Aldolase Deficiency, Fructoseria, Fructosuria, Fryns Syndrome, FSH, FSHD, FSS, Fuchs Dystrophy, Fucosidosis Type 1, Fucosidosis Type 2, Fucosidosis Type 3, Fukuhara Syndrome, Fukuyama Disease, Fukuyama Type Muscular Dystrophy, Fumarylacetoacetase deficiency, Furrowed Tongue, G Syndrome, G6PD Deficiency, G6PD, GA I, GA IIB, GA IIA, GA II, GAII & MADD, Galactorrhea-Amenorrhea Syndrome Nonpuerperal, Galactorrhea-Amenorrhea without Pregnancy, Galactosamine-6-Sulfatase Deficiency, Galactose-1-Phosphate Uridyl Transferase Deficiency, Galactosemia, GALB Deficiency, Galloway-Mowat Syndrome, Galloway Syndrome, GALT Deficiency, Gammaglobulin Deficiency, GAN, Ganglioside Neuraminidase Deficiency, Ganglioside Sialidase Deficiency, Gangliosidosis GM1 Type 1, Gangliosidosis GM2 Type 2, Gangliosidosis Beta Hexosaminidase B Defeciency, Gardner Syndrome, Gargoylism, Garies-Mason Syndrome, Gasser Syndrome, Gastric Intrinsic Factor Failure of Secretion, Enterocyte Cobalamin, Gastrinoma, Gastritis, Gastroesophageal Laceration-Hemorrhage, Gastrointestinal Polyposis and Ectodermal Changes, Gastroschisis, Gaucher Disease, Gaucher-Schlagenhaufer, Gayet-Wernicke Syndrome, GBS, GCA, GCM Syndrome, GCPS, Gee-Herter Disease, Gee-Thaysen Disease, Gehrig's Disease, Gelineau's Syndrome, Genee-Wiedemann Syndrome, Generalized Dystonia, Generalized Familial Neuromyotonia, Generalized Fibromatosis, Generalized Flexion Epilepsy, Generalized Glycogenosis, Generalized Hyperhidrosis, Generalized Lipofuscinosis, Generalized Myasthenia Gravis, Generalized Myotonia, Generalized Sporadic Neuromytonia, Genetic Disorders, Genital Defects, Genital and Urinary Tract Defects, Gerstmann Syndrome, Gerstmann Tetrad, GHBP, GHD, GHR, Giant Axonal Disease, Giant Axonal Neuropathy, Giant Benign Lymphoma, Giant Cell Glioblastoma Astrocytoma, Giant Cell Arteritis, Giant Cell Disease of the Liver, Giant Cell Hepatitis, Giant Cell of Newborns Cirrhosis, Giant Cyst of the Retina, Giant Lymph Node Hyperplasia, Giant Platelet Syndrome Hereditary, Giant Tongue, gic Macular Dystrophy, Gilbert's Disease, Gilbert Syndrome, Gilbert-Dreyfus Syndrome, Gilbert-Lereboullet Syndrome, Gilford Syndrome, Gilles de la Tourette's syndrome, Gillespie Syndrome, Gingival Fibromatosis-Abnormal Fingers Nails Nose Ear Splenomegaly, GLA Deficiency, GLA, GLB1, Glioma Retina, Global aphasia, Globoid Leukodystrophy, Glossoptosis Micrognathia and Cleft Palate, Glucocerebrosidase deficiency, Glucocerebrosidosis, Glucose-6-Phosphate Dehydrogenase Deficiency, Glucose-6-Phosphate Tranport Defect, Glucose-6-Phospate Translocase Deficiency, Glucose-G-Phosphatase Deficiency, Glucose-Galactose Malabsorption, Glucosyl Ceramide Lipidosis, Glutaric Aciduria I, Glutaric Acidemia I, Glutaric Acidemia II, Glutaric Aciduria II, Glutaric Aciduria Type II, Glutaric Aciduria Type III, Glutaricacidemia I, Glutaricacidemia II, Glutaricaciduria I, Glutaricaciduria II, Glutaricaciduria Type IIA, Glutaricaciduria Type IIB, Glutaryl-CoA Dehydrogenase Deficiency, Glutaurate-Aspartate Transport Defect, Gluten-Sensitive Enteropathy, Glycogen Disease of Muscle Type VII, Glycogen Storage Disease I, Glycogen Storage Disease III, Glycogen Storage Disease IV, Glycogen Storage Disease Type V, Glycogen Storage Disease VI, Glycogen Storage Disease VII, Glycogen Storage Disease VIII, Glycogen Storage Disease Type II, Glycogen Storage Disease-Type II, Glycogenosis, Glycogenosis Type I, Glycogenosis Type IA, Glycogenosis Type IB, Glycogenosis Type II, Glycogenosis Type II, Glycogenosis Type III, Glycogenosis Type IV, Glycogenosis Type V, Glycogenosis Type VI, Glycogenosis Type VII, CGlycogenosis Type VIII, Glycolic Aciduria, Glycolipid Lipidosis, GM2 Gangliosidosis Type 1, GM2 Gangliosidosis Type 1, GNPTA, Goitrous Autoimmune Thyroiditis, Goldenhar Syndrome, Goldenhar-Gorlin Syndrome, Goldscheider's Disease, Goltz Syndrome, Goltz-Gorlin Syndrome, Gonadal Dysgenesis 45 X, Gonadal Dysgenesis XO, Goniodysgenesis-Hypodontia, Goodman Syndrome, Goodman, Goodpasture Syndrome, Gordon Syndrome, Gorlin's Syndrome, Gorlin-Chaudhry-Moss Syndrome, Gottron Erythrokeratodermia Congenitalis Progressiva Symmetrica, Gottron's Syndrome, Gougerot-Carteaud Syndrome, Grand Mal Epilepsy, Granular Type Corneal Dystrophy, Granulomatous Arteritis, Granulomatous Colitis, Granulomatous Dermatitis with Eosinophilia, Granulomatous Ileitis, Graves Disease, Graves' Hyperthyroidism, Graves' Disease, Greig Cephalopolysyndactyly Syndrome, Groenouw Type I Corneal Dystrophy, Groenouw Type II Corneal Dystrophy, Gronblad-Strandberg Syndrome, Grotton Syndrome, Growth Hormone Receptor Deficiency, Growth Hormone Binding Protein Deficiency, Growth Hormone Deficiency, Growth-Mental Deficiency Syndrome of Myhre, Growth Retardation-Rieger Anomaly, GRS, Gruber Syndrome, GS, GSD6, GSD8, GTS, Guanosine Triphosphate-Cyclohydrolase Deficiency, Guanosine Triphosphate-Cyclohydrolase Deficiency, Guenther Porphyria, Guerin-Stern Syndrome, Guillain-Barré, Guillain-Barre Syndrome, Gunther Disease, H Disease, H. Gottron's Syndrome, Habit Spasms, HAE, Hageman Factor Deficiency, Hageman factor, Haim-Munk Syndrome, Hajdu-Cheney Syndrome, Hajdu Cheney, HAL Deficiency, Hall-Pallister Syndrome, Hallermann-Streiff-Francois syndrome, Hallermann-Streiff Syndrome, Hallervorden-Spatz Disease, Hallervorden-Spatz Syndrome, Hallopeau-Siemens Disease, Hallux Duplication Postaxial Polydactyly and Absence of Corpus Callosum, Halushi-Behcet's Syndrome, Hamartoma of the Lymphatics, Hand-Schueller-Christian Syndrome, HANE, Hanhart Syndrome, Happy Puppet Syndrome, Harada Syndrome, HARD ±E Syndrome, HARD Syndrome, Hare Lip, Harlequin Fetus, Harlequin Type DOC 6, Harlequin Type Ichthyosis, Harley Syndrome, Harrington Syndrome, Hart Syndrome, Hartnup Disease, Hartnup Disorder, Hartnup Syndrome, Hashimoto's Disease, Hashimoto-Pritzker Syndrome, Hashimoto's Syndrome, Hashimoto's Thyroiditis, Hashimoto-Pritzker Syndrome, Hay Well's Syndrome, Hay-Wells Syndrome of Ectodermal Dysplasia, HCMM, HCP, HCTD, HD, Heart-Hand Syndrome (Holt-Oram Type), Heart Disease, Hecht Syndrome, HED, Heerferdt-Waldenstrom and Lofgren's Syndromes, Hegglin's Disease, Heinrichsbauer Syndrome, Hemangiomas, Hemangioma Familial, Hemangioma-Thrombocytopenia Syndrome, Hemangiomatosis Chondrodystrophica, Hemangiomatous Branchial Clefts-Lip Pseudocleft Syndrome, Hemifacial Microsomia, Hemimegalencephaly, Hemiparesis of Cerebral Palsy, Hemiplegia of Cerebral Palsy, Hemisection of the Spinal Cord, Hemochromatosis, Hemochromatosis Syndrome, Hemodialysis-Related Amyloidosis, Hemoglobin Lepore Syndromes, Hemolytic Anemia of Newborn, Hemolytic Cold Antibody Anemia, Hemolytic Disease of Newborn, Hemolytic-Uremic Syndrome, Hemophilia, Hemophilia A, Hemophilia B, Hemophilia B Factor IX, Hemophilia C, Hemorrhagic Dystrophic Thrombocytopenia, Hemorrhagica Aleukia, Hemosiderosis, Hepatic Fructokinase Deficiency, Hepatic Phosphorylase Kinase Deficiency, Hepatic Porphyria, Hepatic Porphyrias, Hepatic Veno-Occlusive Diseas, Hepato-Renal Syndrome, Hepatolenticular Degeneration, Hepatophosphorylase Deficiency, Hepatorenal Glycogenosis, Hepatorenal Syndrome, Hepatorenal Tyrosinemia, Hereditary Acromelalgia, Hereditary Alkaptonuria, Hereditary Amyloidosis, Hereditary Angioedema, Hereditary Areflexic Dystasia, Heredopathia Atactica Polyneuritiformis, Hereditary Ataxia, Hereditary Ataxia Friedrich's Type, Hereditary Benign Acanthosis Nigricans, Hereditary Cerebellar Ataxia, Hereditary Chorea, Hereditary Chronic Progressive Chorea, Hereditary Connective Tissue Disorders, Hereditary Coproporphyria, Hereditary Coproporphyria Porphyria, Hereditary Cutaneous Malignant Melanoma, Hereditary Deafness-Retinitis Pigmentosa, Heritable Disorder of Zinc Deficiency, Hereditary DNS, Hereditary Dystopic Lipidosis, Hereditary Emphysema, Hereditary Fructose Intolerance, Hereditary Hemorrhagic Telangiectasia, Hereditary Hemorrhagic Telangiectasia Type I, Hereditary Hemorrhagic Telangiectasia Type H, Hereditary Hemorrhagic Telangiectasia Type III, Hereditary Hyperuricemia and Choreoathetosis Syndrome, Hereditary Leptocytosis Major, Hereditary Leptocytosis Minor, Hereditary Lymphedema, Hereditary Lymphedema Tarda, Hereditary Lymphedema Type I, Hereditary Lymphedema Type II, Hereditary Motor Sensory Neuropathy, Hereditary Motor Sensory Neuropathy I, Hereditary Motor Sensory Neuropathy Type III, Hereditary Nephritis, Hereditary Nephritis and Nerve Deafness, Hereditary Nephropathic Amyloidosis, Hereditary Nephropathy and Deafness, Hereditary Nonpolyposis Colorectal Cancer, Hereditary Nonpolyposis Colorectal Carcinoma, Hereditary Nonspherocytic Hemolytic Anemia, Hereditary Onychoosteodysplasia, Hereditary Optic Neuroretinopathy, Hereditary Polyposis Coli, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Hereditary Site Specific Cancer, Hereditary Spherocytic Hemolytic Anemia, Hereditary Spherocytosis, Hereditary Tyrosinemia Type 1, Heritable Connective Tissue Disorders, Herlitz Syndrome, Hermans-Herzberg Phakomatosis, Hermansky-Pudlak Syndrome, Hermaphroditism, Herpes Zoster, Herpes Iris Stevens-Johnson Type, Hers Disease, Heterozygous Beta Thalassemia, Hexoaminidase Alpha-Subunit Deficiency (Variant B), Hexoaminidase Alpha-Subunit Deficiency (Variant B), HFA, HFM, HOPS, HH, HHHO, HHRH, HHT, Hiatal Hernia-Microcephaly-Nephrosis Galloway Type, Hidradenitis Suppurativa, Hidrosadenitis Axillaris, Hidrosadenitis Suppurativa, Hidrotic Ectodermal Dysplasias, HIE Syndrome, High Imperforate Anus, High Potassium, High Scapula, HIM, Hirschsprung's Disease, Hirschsprung's Disease Acquired, Hirschsprung Disease Polydactyly of Ulnar & Big Toe and VSD, Hirschsprung Disease with Type D Brachydactyly, Hirsutism, HIS Deficiency, Histidine Ammonia-Lyase (HAL) Deficiency, Histidase Deficiency, Histidinemia, Histiocytosis, Histiocytosis X, HLHS, HLP Type II, HMG, HMI, HMSN I, HNHA, HOCM, Hodgkin Disease, Hodgkin's Disease, Hodgkin's Lymphoma, Hollaender-Simons Disease, Holmes-Adie Syndrome, Holocarboxylase Synthetase Deficiency, Holoprosencephaly, Holoprosencephaly Malformation Complex, Holoprosencephaly Sequence, Holt-Oram Syndrome, Holt-Oram Type Heart-Hand Syndrome, Homocystinemia, Homocystinuria, Homogentisic Acid Oxidase Deficiency, Homogentisic Acidura, Homozygous Alpha-1-Antitrypsin Deficiency, HOOD, Homer Syndrome, Horton's disease, HOS, HOS1, Houston-Harris Type Achrondrogenesis (Type IA), HPS, HRS, HS, HSAN Type I, HSAN Type II, HSAN-III, HSMN, HSMN Type III, HSN I, HSN-III, Huebner-Herter Disease, Hunner's Patch, Hunner's Ulcer, Hunter Syndrome, Hunter-Thompson Type Acromesomelic Dysplasia, Huntington's Chorea, Huntington's Disease, Hurler Disease, Hurler Syndrome, Hurler-Scheie Syndrome, HUS, Hutchinson-Gilford Progeria Syndrome, Hutchinson-Cilford Syndrome, Hutchinson-Weber-Peutz Syndrome, Hutterite Syndrome Bowen-Conradi Type, Hyaline Panneuropathy, Hydranencephaly, Hydrocephalus, Hydrocephalus Agyria and Retinal Dysplasia, Hydrocephalus Internal Dandy-Walker Type, Hydrocephalus Noncommunicating Dandy-Walker Type, Hydrocephaly, Hydronephrosis With Peculiar Facial Expression, Hydroxylase Deficiency, Hygroma Colli, Hyper-IgE Syndrome, Hyper-IgM Syndrome, Hyperaldosteronism, Hyperaldosteronism With Hypokalemic Alkatosis, Hyperaldosteronism Without Hypertension, Hyperammonemia, Hyperammonemia Due to Carbamylphosphate Synthetase Deficiency, Hyperammonemia Due to Ornithine Transcarbamylase Deficiency, Hyperammonemia Type II, Hyper-Beta Carnosinemia, Hyperbilirubinemia I, Hyperbilirubinemia II, Hypercalcemia Familial with Nephrocalcinosis and Indicanuria, Hypercalcemia-Supravalvar Aortic Stenosis, Hypercalciuric Rickets, Hypercapnic acidosis, Hypercatabolic Protein-Losing Enteropathy, Hyperchloremic acidosis, Hypercholesterolemia, Hypercholesterolemia Type IV, Hyperchylomicronemia, Hypercystinuria, Hyperekplexia, Hyperextensible joints, Hyperglobulinemic Purpura, Hyperglycinemia with Ketoacidosis and Lactic Acidosis Propionic Type, Hyperglycinemia Nonketotic, Hypergonadotropic Hypogonadism, Hyperimmunoglobulin E Syndrome, Hyperimmunoglobulin E-Recurrent Infection Syndrome, Hyperimmunoglobulinemia E-Staphylococcal, Hyperkalemia, Hyperkinetic Syndrome, Hyperlipemic Retinitis, Hyperlipidemia I, Hyperlipidemia IV, Hyperlipoproteinemia Type I, Hyperlipoproteinemia Type III, Hyperlipoproteinemia Type IV, Hyperoxaluria, Hyperphalangy-Clinodactyly of Index Finger with Pierre Robin Syndrome, Hyperphenylalanemia, Hyperplastic Epidermolysis Bullosa, Hyperpnea, Hyperpotassemia, Hyperprebeta-Lipoproteinemia, Hyperprolinemia Type I, Hyperprolinemia Type II, Hypersplenism, Hypertelorism with Esophageal Abnormalities and Hypospadias, Hypertelorism-Hypospadias Syndrome, Hypertrophic Cardio myopathy, Hypertrophic Interstitial Neuropathy, Hypertrophic Interstitial Neuritis, Hypertrophic Interstitial Radiculoneuropathy, Hypertrophic Neuropathy of Refsum, Hypertrophic Obstructive Cardio myopathy, Hyperuricemia Choreoathetosis Self-multilation Syndrome, Hyperuricemia-Oligophrenia, Hypervalinemia, Hypocalcified (Hypomineralized) Type, Hypochondrogenesis, Hypochrondroplasia, Hypogammaglobulinemia, Hypogammaglobulinemia Transient of Infancy, Hypogenital Dystrophy with Diabetic Tendency, Hypoglossia-Hypodactylia Syndrome, Hypoglycemia, Exogenous Hypoglycemia, Hypoglycemia with Macroglossia, Hypoglycosylation Syndrome Type la, Hypoglycosylation Syndrome Type 1a, Hypogonadism with Anosmia, Hypogonadotropic Hypogonadism and Anosmia, Hypohidrotic Ectodermal Dysplasia, Hypohidrotic Ectodermal Dysplasia Autosomal Dominant type, Hypohidrotic Ectodermal Dysplasias Autorecessive, Hypokalemia, Hypokalemic Alkalosis with Hypercalciuria, Hypokalemic Syndrome, Hypolactasia, Hypomaturation Type (Snow-Capped Teeth), Hypomelanosis of Ito, Hypomelia-Hypotrichosis-Facial Hemangioma Syndrome, Hypomyelination Neuropathy, Hypoparathyroidism, Hypophosphatasia, Hypophosphatemic Rickets with Hypercalcemia, Hypopigmentation, Hypopigmented macular lesion, Hypoplasia of the Depressor Anguli Oris Muscle with Cardiac Defects, Hypoplastic Anemia, Hypoplastic Congenital Anemia, Hypoplastic Chondrodystrophy, Hypoplastic Enamel-Onycholysis-Hypohidrosis, Hypoplastic (Hypoplastic-Explastic) Type, Hypoplastic Left Heart Syndrome, Hypoplastic-Triphalangeal Thumbs, Hypopotassemia Syndrome, Hypospadias-Dysphagia Syndrome, Hyposmia, Hypothalamic Hamartoblastoma Hypopituitarism Imperforate Anus Polydactyly, Hypothalamic Infantilism-Obesity, Hypothyroidism, Hypotonia-Hypomentia-Hypogonadism-Obesity Syndrome, Hypoxanthine-Guanine Phosphoribosyltranferase Defect (Complete Absense of), I-Cell Disease, Iatrogenic Hypoglycemia, IBGC, IBIDS Syndrome, IBM, IBS, IC, I-Cell Disease, ICD, ICFE Syndrome Cogan-Reese Type, Icelandic Type Amyloidosis (Type VI), I-Cell Disease, Ichthyosiform Erythroderma Corneal Involvement and Deafness, Ichthyosiform Erythroderma Hair Abnormality Growth and Men, Ichthyosiform Erythroderma with Leukocyte Vacuolation, Ichthyosis, Ichthyosis Congenita, Ichthyosis Congenital with Trichothiodystrophy, Ichthyosis Hystrix, Ichthyosis Hystrix Gravior, Ichthyosis Linearis Circumflexa, Ichthyosis Simplex, Ichthyosis Tay Syndrome, Ichthyosis Vulgaris, Ichthyotic Neutral Lipid Storage Disease, Icteric Leptospirosis, Icterohemorrhagic Leptospirosis, Icterus (Chronic Familial), Icterns Gravis Neonatorum, Icterus Intermittens Juvenalis, Idiopathic Alveolar Hypoventilation, Idiopathic Amyloidosis, Idiopathic Arteritis of Takayasu, Idiopathic Basal Ganglia Calcification (I13GC), Idiopathic Brachial Plexus Neuropathy, Idiopathic Cervical Dystonia, Idiopathic Dilatation of the Pulmonary Artery, Idiopathic Facial Palsy, Idiopathic Familial Hyperlipemia, Idiopathic Hypertrophic Subaortic Stenosis, Idiopathic Hypoproteinemia, Idiopathic Immunoglobulin Deficiency, Idiopathic Neonatal Hepatitis, Idiopathic Non-Specific Ulcerative Colitis, Idiopathic Peripheral Periphlebitis, Idiopathic Pulmonary Fibrosis, Idiopathic Refractory Sideroblastic Anemia, Idiopathic Renal Hematuria, Idiopathic Steatorrhea, Idiopathic Thrombocythemia, Idiopathic Thrombocytopenic Purpura, Idiopathic Thrombocytopenia Purpura (ITP), IDPA, IgA Nephropathy, IHSS, Ileitis, Ileocolitis, Illinois Type Amyloidosis, ILS, IM, IMD2, IMD5, Immune Defect due to Absence of Thymus, Immune Hemolytic Anemia Paroxysmal Cold, Immunodeficiency with Ataxia Telangiectasia, Immunodeficiency Cellular with Abnormal Immunoglobulin Synthesis, Immunodeficiency Common Variable Unclassifiable, Immunodeficiency with Hyper-IgM, Immunodeficiency with Leukopenia, Immunodeficiency-2, Immunodeficiency-5 (IMD5), Immunoglobulin Deficiency, Imperforate Anus, Imperforate Anus with Hand Foot and Ear Anomalies, Imperforate Nasolacrimal Duct and Premature Aging Syndrome, Impotent Neutrophil Syndrome, Inability To Open Mouth Completely And Short Finger-Flexor, INAD, Inborn Error of Urea Synthesis Arginase Type, Inborn Error of Urea Synthesis Arginino Succinic Type, Inborn Errors of Urea Synthesis Carbamyl Phosphate Type, Inborn Error of Urea Synthesis Citrullinemia Type, Inborn Errors of Urea Synthesis Glutamate Synthetase Type, INCL, Inclusion body myositis, Incomplete Atrioventricular Septal Defect, Incomplete Testicular Feminization, Incontinentia Pigmenti, Incontinenti Pigmenti Achromians, Index Finger Anomaly with Pierre Robin Syndrome, Indiana Type Amyloidosis (Type II), Indolent systemic mastocytosis, Infantile Acquired Aphasia, Infantile Autosomal Recessive Polycystic Kidney Disease, Infantile Beriberi, Infantile Cerebral Ganglioside, Infantile Cerebral Paralysis, Infantile Cystinosis, Infantile Epileptic, Infantile Fanconi Syndrome with Cystinosis, Infantile Finnish Type Neuronal Ceroid Lipofuscinosis, Infantile Gaucher Disease, Infantile Hypoglycemia, Infantile Hypophasphatasia, Infantile Lobar Emphysema, Infantile Myoclonic Encephalopathy, Infantile Myoclonic Encephalopathy and Polymyoclonia, Infantile Myofibromatosis, Infantile Necrotizing Encephalopathy, Infantile Neuronal Ceroid Lipofuscinosis, Infantile Neuroaxonal Dystrophy, Infantile Onset Schindler Disease, Infantile Phytanic Acid Storage Disease, Infantile Refsum Disease (IRD), Infantile Sipoidosis GM-2 Gangliosideosis (Type S), Infantile Sleep Apnea, Infantile Spasms, Infantile Spinal Muscular Atrophy (all types), Infantile Spinal Muscular Atrophy ALS, Infantile Spinal Muscular Atrophy Type I, Infantile Type Neuronal Ceroid Lipofuscinosis, Infectious Jaundice, Inflammatory Breast Cancer, Inflammatory Linear Nevus Sebaceous Syndrome, Iniencephaly, Insulin Resistant Acanthgsis Nigricans, Insulin Lipodystrophy, Insulin dependent Diabetes, Intention Myoclonus, Intermediate Cystinosis, Intermediate Maple Syrup Urine Disease, Intermittent Ataxia with Pyruvate Dehydrogenase Deficiency, Intermittent Maple Syrup Urine Disease, Internal Hydrocephalus, Interstitial Cystitis, Interstitial Deletion of 4q Included, Intestinal Lipodystrophy, Intestinal Lipophagic Granulomatosis, Intestinal Lymphangiectasia, Intestinal Polyposis I, Intestinal Polyposis II, Intestinal Polyposis III, Intestinal Polyposis-Cutaneous Pigmentation Syndrome, Intestinal Pseudoobstruction with External Ophthalmoplegia, Intracranial Neoplasm, Intracranial Tumors, Intracranial Vascular Malformations, Intrauterine Dwarfism, Intrauterine Synechiae, Inverted Smile And Occult Neuropathic Bladder, Iowa Type Amyloidosis (Type IV), IP, IPA, Iridocorneal Endothelial Syndrome, Iridocorneal Endothelial (ICE) Syndrome Cogan-Resse Type, Iridogoniodysgenesis With Somatic Anomalies, Iris Atrophy with Corneal Edema and Glaucoma, Iris Nevus Syndrome, Iron Overload Anemia, Iron Overload Disease, Irritable Bowel Syndrome, Irritable Colon Syndrome, Isaacs Syndrome, Isaacs-Merten Syndrome, Ischemic Cardio myopathy, Isolated Lissencephaly Sequence, Isoleucine 33 Amyloidosis, Isovaleric Acid CoA Dehydrogenase Deficiency, Isovaleric Acidaemia, Isovalericacidemia, Isovaleryl CoA Carboxylase Deficiency, ITO Hypomelanosis, ITO, ITP, IVA, Ivemark Syndrome, Iwanoff Cysts, Jackknife Convulsion, Jackson-Weiss Craniosynostosis, Jackson-Weiss Syndrome, Jacksonian Epilepsy, Jacobsen Syndrome, Jadassohn-Lewandowsky Syndrome, Jaffe-Lichenstein Disease, Jakob's Disease, Jakob-Creutzfeldt Disease, Janeway I, Janeway Dysgammaglobulinemia, Jansen Metaphyseal Dysostosis, Jansen Type Metaphyseal Chondrodysplasia, Jarcho-Levin Syndrome, Jaw-Winking, JBS, JDMS, Jegher's Syndrome, Jejunal Atresia, Jejunitis, Jejunoileitis, Jervell and Lange-Nielsen Syndrome, Jeune Syndrome, JMS, Job Syndrome, Job-Buckley Syndrome, Johanson-Blizzard Syndrome, John Dalton, Johnson-Stevens Disease, Jonston's Alopecia, Joseph's Disease, Joseph's Disease Type I, Joseph's Disease Type II, Joseph's Disease Type III, Joubert Syndrome, Joubert-Bolthauser Syndrome, JRA, Juberg Hayward Syndrome, Juberg-Marsidi Syndrome, Juberg-Marsidi Mental Retardation Syndrome, Jumping Frenchmen, Jumping Frenchmen of Maine, Juvenile Arthritis, Juvenile Autosomal Recessive Polycystic Kidney Disease, Juvenile Cystinosis, Juvenile (Childhood) Dermatomyositis (JDMS), Juvenile Diabetes, Juvenile Gaucher Disease, Juvenile Gout Choreoathetosis and Mental Retardation Syndrome, Juvenile Intestinal Malabsorption of Vit B12, Juvenile Intestinal Malabsorption of Vitamin B12, Juvenile Macular Degeneration, Juvenile Pernicious Anemia, Juvenile Retinoschisis, Juvenile Rheumatoid Arthritis, Juvenile Spinal Muscular Atrophy Included, Juvenile Spinal Muscular Atrophy AILS Included, Juvenile Spinal Muscular Atrophy Type III, Juxta-Articular Adiposis Dolorosa, Juxtaglomerular Hyperplasia, Kabuki Make-Up Syndrome, Kahler Disease, Kallmann Syndrome, Kanner Syndrome, Kanzaki Disease, Kaposi Disease (not Kaposi Sarcoma), Kappa Light Chain Deficiency, Karsch-Neugebauer Syndrome, Kartagener Syndrome-Chronic Sinobronchial Disease and Dextrocardia, Kartagener Triad, Kasabach-Merritt Syndrome, Kast Syndrome, Kawasaki Disease, Kawasaki Syndrome, KBG Syndrome, KD, Kearns-Sayre Disease, Kearns-Sayre Syndrome, Kennedy Disease, Kennedy Syndrome, Kennedy Type Spinal and Bulbar Muscular Atrophy, Kennedy-Stefanis Disease, Kenny Disease, Kenny Syndrome, Kenny Type Tubular Stenosis, Kenny-Caffe Syndrome, Kera. Palmoplant. Con. Pes Planus Ony. Periodon. Arach., Keratitis Ichthyosis Deafness Syndrome, Keratoconus, Keratoconus Posticus Circumscriptus, Keratolysis, Keratolysis Exfoliativa Congenita, Keratolytic Winter Erythema, Keratomalacia, Keratosis Follicularis, Keratosis Follicularis Spinulosa Decalvans, Keratosis Follicularis Spinulosa Decalvans Ichthyosis, Keratosis Nigricans, Keratosis Palmoplantaris with Periodontopathia and Onychogryposis, Keratosis Palmoplantaris Congenital Pes Planus Onychogryposis Periodontosis Arachnodactyly, Keratosis Palmoplantaris Congenital, Pes Planus, Onychogryphosis, Periodontosis, Arachnodactyly, Acroosteolysis, Keratosis Rubra Figurata, Keratosis Seborrheica, Ketoacid Decarboxylase Deficiency, Ketoaciduria, Ketotic Glycinemia, KFS, KID Syndrome, Kidney Agenesis, Kidneys Cysfic-Retinal Aplasia Joubert Syndrome, Killian Syndrome, Killian/Teschler-Nicola Syndrome, Kiloh-Nevin syndrome III, Kinky Hair Disease, Kinsboume Syndrome, Kleeblattschadel Deformity, Kleine-Levin Syndrome, Kleine-Levin Hibernation Syndrome, Klinefelter, Klippel-Feil Syndrome, Klippel-Feil Syndrome Type I, Klippel-Feil Syndrome Type II, Klippel-Feil Syndrome Type III, Klippel Trenaunay Syndrome, Klippel-Trenaunay-Weber Syndrome, Kluver-Bucy Syndrome, KMS, Kniest Dysplasia, Kniest Syndrome, Kobner's Disease, Koebberling-Dunnigan Syndrome, Kohlmeier-Degos Disease, Kok Disease, Korsakoff Psychosis, Korsakoff's Syndrome, Krabbe's Disease Included, Krabbe's Leukodystrophy, Kramer Syndrome, KSS, KTS, KTW Syndrome, Kufs Disease, Kugelberg-Welander Disease, Kugelberg-Welander Syndrome, Kussmaul-Landry Paralysis, KWS, L-3-Hydroxy-Acyl-CoA Dehydrogenase (LCHAD) Deficiency, Laband Syndrome, Labhart-Willi Syndrome, Labyrinthine Syndrome, Labyrinthine Hydrops, Lacrimo-Auriculo-Dento-Digital Syndrome, Lactase Isolated Intolerance, Lactase Deficiency, Lactation-Uterus Atrophy, Lactic Acidosis Leber Hereditary Optic Neuropathy, Lactic and Pyruvate Acidemia with Carbohydrate Sensitivity, Lactic and Pyruvate Acidemia with Episodic Ataxia and Weakness, Lactic and Pyruvate, Lactic acidosis, Lactose Intolerance of Adulthood, Lactose Intolerance, Lactose Intolerance of Childhood, LADD Syndrome, LADD, Lafora Disease Included, Lafora Body Disease, Laki-Lorand Factor Deficiency, LAM, Lambert Type Ichthyosis, Lambert-Eaton Syndrome, Lambert-Eaton Myasthenic Syndrome, Lamellar Recessive Ichthyosis, Lamellar Ichthyosis, Lancereaux-Mathieu-Weil Spirochetosis, Landau-Kleffner Syndrome, Landouzy Dejerine Muscular Dystrophy, Landry Ascending Paralysis, Langer-Salidino Type Achondrogensis (Type II), Langer Giedion Syndrome, Langerhans-Cell Granulomatosis, Langerhans-Cell Histiocytosis (LCH), Large Atrial and Ventricular Defect, Laron Dwarfism, Laron Type Pituitary Dwarfism, Larsen Syndrome, Laryngeal Dystonia, Latah (Observed in Malaysia), Late Infantile Neuroaxonal Dystrophy, Late Infantile Neuroaxonal Dystrophy, Late Onset Cockayne Syndrome Type III (Type C), Late-Onset Dystonia, Late-Onset linmunoglobulin Deficiency, Late Onset Pelizaeus-Merzbacher Brain Sclerosis, Lattice Corneal Dystrophy, Lattice Dystrophy, Launois-Bensaude, Launois-Cleret Syndrome, Laurence Syndrome, Laurence-Moon Syndrome, Laurence-Moon/Bardet-Biedl, Lawrence-Seip Syndrome, LCA, LCAD Deficiency, LCAD, LCAD, LCADH Deficiency, LCH, LCHAD, LCPD, Le Jeune Syndrome, Leband Syndrome, Leber's Amaurosis, Leber's Congenital Amaurosis, Congenital Absence of the Rods and Cones, Leber's Congenital Tapetoretinal Degeneration, Leber's Congenital Tapetoretinal Dysplasia, Leber's Disease, Leber's Optic Atrophy, Leber's Optic Neuropathy, Left Ventricular Fibrosis, Leg Ulcer, Legg-Calve-Perthes Disease, Leigh's Disease, Leigh's Syndrome, Leigh's Syndrome (Subacute Necrotizing Encephalomyelopathy), Leigh Necrotizing Encephalopathy, Lennox-Gastaut Syndrome, Lentigio-Polypose-Digestive Syndrome, Lenz Dysmorphogenetic Syndrome, Lenz Dysplasia, Lenz Microphthalmia Syndrome, Lenz Syndrome, LEOPARD Syndrome, Leprechaunism, Leptomeningeal Angiomatosis, Leptospiral Jaundice, Leri-Weill Disease, Leri-Weil Dyschondrosteosis, Leri-Weil Syndrome, Lermoyez Syndrome, Leroy Disease, Lesch Nyhan Syndrome, Lethal Infantile Cardio myopathy, Lethal Neonatal Dwarfism, Lethal Osteochondrodysplasia, Letterer-Siwe Disease, Leukocytic Anomaly Albinism, Leukocytic Inclusions with Platelet Abnormality, Leukodystrophy, Leukodystrophy with Rosenthal Fibers, Leukoencephalitis Periaxialis Concentric, Levine-Critchley Syndrome, Levulosuria, Levy-Hollister Syndrome, LGMD, LGS, LHON, LIC, Lichen Ruber Acuminatus, Lichen Acuminatus, Lichen Amyloidosis, Lichen Planus, Lichen Psoriasis, Lignac-Debre-Fanconi Syndrome, Lignac-Fanconi Syndrome, Ligneous Conjunctivitis, Limb-Girdle Muscular Dystrophy, Limb Malformations-Dento-Digital Syndrome, Limit Dextrinosis, Linear Nevoid Hypermelanosis, Linear Nevus Sebacous Syndrome, Linear Scleroderma, Linear Sebaceous Nevus Sequence, Linear Sebaceous Nevus Syndrome, Lingua Fissurata, Lingua Plicata, Lingua Scrotalis, Linguofacial Dyskinesia, Lip Pseudocleft-hemangiomatous Branchial Cyst Syndrome, Lipid Granulomatosis, Lipid Histiocytosis, Lipid Kerasin Type, Lipid Storage Disease, Lipid-Storage myopathy Associated with SCAD Deficiency, Lipidosis Ganglioside Infantile, Lipoatrophic Diabetes Mellitus, Lipodystrophy, Lipoid Corneal Dystrophy, Lipoid Hyperplasia-Male Pseudohermaphroditism, Lipomatosis of Pancreas Congenital, Lipomucopolysaccharidosis Type I, Lipomyelomeningocele, Lipoprotein Lipase Deficiency Familial, LIS, LIS1, Lissencephaly 1, Lissencephaly Type I, Lissencephaly variants with agenesis of the corpus callosum cerebellar hypoplasia or other anomalies, Little Disease, Liver Phosphorylase Deficiency, LKS, LM Syndrome, Lobar Atrophy, Lobar Atrophy of the Brain, Lobar Holoprosencephaly, Lobar Tension Emphysema in Infancy, Lobstein Disease (Type I), Lobster Claw Deformity, Localized Epidermolysis Bullosa, Localized Lipodystrophy, Localized Neuritis of the Shoulder Girdle, Loeffler's Disease, Loeffler Endomyocardial Fibrosis with Eosinophilia, Loeffler Fibroplastic Parietal Endocarditis, Loken Syndrome, Loken-Senior Syndrome, Long-Chain 3-hydroxyacyl-CoA Dehydrogenase (LCHAD), Long Chain Acyl CoA Dehydrogenase Deficiency, Long-Chain Acyl-CoA Dehydrogenase (ACADL), Long-Chain Acyl-CoA Dehydrogenase Deficiency, Long QT Syndrome without Deafness, Lou Gehrig's Disease, Lou Gehrig's Disease Included, Louis-Bar Syndrome, Low Blood Sugar, Low-Density Beta Lipoprotein Deficiency, Low Imperforate Anus, Low Potassium Syndrome, Lowe syndrome, Lowe's Syndrome, Lowe-Bickel Syndrome, Lowe-Terry-MacLachlan Syndrome, LS, LTD), Lubs Syndrome, Luft Disease, Lumbar Canal Stenosis, Lumbar Spinal Stenosis, Lumbosacral Spinal Stenosis, Lundborg-Unverricht Disease, Lundborg-Unverricht Disease Included, Lupus, Lupus, Lupus Erythematosus, Luschka-Magendie Foramina Atresia, Lyell Syndrome, Lyelles Syndrome, Lymphadenoid Goiter, Lymphangiectatic Protein-Losing Enteropathy, Lymphangioleiomatosis, Lymphangioleimyomatosis, Lymphangiomas, Lymphatic Malformations, Lynch Syndromes, Lynch Syndrome I, Lynch Syndrome II, Lysosomal Alpha-N-Acetylgalactosaminidase Deficiency Schindler Type, Lysosomal Glycoaminoacid Storage Disease-Angiokeratoma Corporis Diffusum, Lysosomal Glucosidase Deficiency, MAA, Machado Disease, Machado-Joseph Disease, Macrencephaly, Macrocephaly, Macrocephaly Hemihypertrophy, Macrocephaly with Multiple Lipomas and Hemangiomata, Macrocephaly with Pseudopapilledema and Multiple Hemangiomata, Macroglobulinemia, Macroglossia, Macroglossia-Omphalocele-Visceromegaly Syndrome, Macrostomia Ablepheron Syndrome, Macrothrombocytopenia Familial Bernard-Soulier Type, Macula Lutea degeneration, Macular Amyloidosis, Macular Degeneration, Macular Degeneration Disciform, Macular Degeneration Senile, Macular Dystrophy, Macular Type Corneal Dystrophy, MAD, Madelung's Disease, Maffucci Syndrome, Major Epilepsy, Malabsorption, Malabsorption-Ectodermal Dysplasia-Nasal Alar Hypoplasia, Maladie de Roger, Maladie de Tics, Male Malformation of Limbs and Kidneys, Male Turner Syndrome, Malignant Acanthosis, Malignant Acanthosis Nigricans, Malignant Astrocytoma, Malignant Atrophic Papulosis, Malignant Fever, Malignant Hyperphenylalaninemia, Malignant Hyperpyrexia, Malignant Hyperthermia, Malignant Melanoma, Malignant Tumors of the Central Nervous System, Mallory-Weiss Laceration, Mallory-Weiss Tear, Mallory-Weiss Syndrome, Mammary Paget's Disease, Mandibular Ameloblastoma, Mandibulofacial Dysostosis, Mannosidosis, Map-Dot-Fingerprint Type Corneal Dystrophy, Maple Syrup Urine Disease, Marble Bones, Marchiafava-Micheli Syndrome, Marcus Gunn Jaw-Winking Syndrome, Marcus Gunn Phenomenon, Marcus Gunn Ptosis with jaw-winking, Marcus Gunn Syndrome, Marcus Gunn (Jaw-Winking) Syndrome, Marcus (lunn Ptosis (with jaw-winking), Marden-Walker Syndrome, Marden-Walker Type Connective Tissue Disorder, Marfan's Abiotrophy, Marfan-Achard syndrome, Marfan Syndrome, Marfan's Syndrome I, Marfan's Variant, Marfanoid Hypermobility Syndrome, Marginal Corneal Dystrophy, Marie's Ataxia, Marie Disease, Marie-Sainton Disease, Marie Strumpell Disease, Marie-Strumpell Spondylitis, Marinesco-Sjogren Syndrome, Marinesco-Sjogren-Gorland Syndrome, Marker X Syndrome, Maroteaux Lamy Syndrome, Maroteaux Type Acromesomelic Dysplasia, Marshall's Ectodermal Dysplasias With Ocular and Hearing Defects, Marshall-Smith Syndrome, Marshall Syndrome, Marshall Type Deafness-Myopia-Cataract-Saddle Nose, Martin-Albright Syndrome, Martin-Bell Syndrome, Martorell Syndrome, MASA Syndrome, Massive Myoclonia, Mast Cell Leukemia, Mastocytosis, Mastocytosis With an Associated Hematologic Disorder, Maumenee Corneal Dystrophy, Maxillary Ameloblastoma, Maxillofacial Dysostosis, Maxillonasal Dysplasia, Maxillonasal Dysplasia Binder Type, Maxillopalpebral Synkinesis, May-Hegglin Anomaly, MCAD Deficiency, MCAD, McArdle Disease, McCune-Albright, MCD, McKusick Type Metaphyseal Chondrodysplasia, MCR, MCTD, Meckel Syndrome, Meckel-Gruber Syndrome, Median Cleft Face Syndrome, Mediterranean Anemia, Medium-Chain Acyl-CoA dehydrogenase (ACADM), Medium Chain Acyl-CoA Dehydrogenase (MCAD) Deficiency, Medium-Chain Acyl-CoA Dehydrogenase Deficiency, Medullary Cystic Disease, Medullary Sponge Kidney, MEF, Megaesophagus, Megalencephaly, Megalencephaly with Hyaline Inclusion, Megalencephaly with Hyaline Panneuropathy, Megaloblastic Anemia, Megaloblastic Anemia of Pregnancy, Megalocornea-Mental Retardation Syndrome, Meier-Gorlin Syndrome, Meige's Lymphedema, Meige's Syndrome, Melanodermic Leukodystrophy, Melanoplakia-Intestinal Polyposis, Melanoplakia-Intestinal Polyposis, MELAS Syndrome, MELAS, Melkersson Syndrome, Melnick-Fraser Syndrome, Melnick-Needles Osteodysplasty, Melnick-Needles Syndrome, Membranous Lipodystrophy, Mendes Da Costa Syndrome, Meniere Disease, Ménière's Disease, Meningeal Capillary Angiomatosis, Menkes Disease, Menke's Syndrome I, Mental Retardation Aphasia Shuffling Gait Adducted Thumbs (MASA), Mental Retardation-Deafness-Skeletal Abnormalities-Coarse Face with Full Lips, Mental Retardation with Hypoplastic 5th Fingernails and Toenails, Mental Retardation with Osteocartilaginous Abnormalities, Mental Retradation-X-linked with Growth Delay-Deafness-Microgenitalism, Menzel Type OPCA, Mermaid Syndrome, MERRF, MERRF Syndrome, Merten-Singleton Syndrome, MES, Mesangial IGA Nephropathy, Mesenteric Lipodystrophy, Mesiodens-Cataract Syndrome, Mesodermal Dysmorphodystrophy, Mesomelic Dwarfism-Madelung Deformity, Metabolic Acidosis, Metachromatic Leukodystrophy, Metatarsus Varus, Metatropic Dwarfism Syndrome, Metatropic Dysplasia, Metatropic Dysplasia I, Metatropic Dysplasia II, Methylmalonic Acidemia, Methylmalonic Aciduria, Meulengracht's Disease, MFD1, MG, MH, MHA, Micrencephaly, Microcephalic Primordial Dwarfism I, Microcephaly, Microcephaly-Hiatal Hernia-Nephrosis Galloway Type, Microcephaly-Hiatal Hernia-Nephrotic Syndrome, Microcystic Corneal Dystrophy, Microcythemia, Microlissencephaly, Microphthalmia, Microphthalmia or Anophthalmos with Associated Anomalies, Micropolygyria With Muscular Dystrophy, Microtia Absent Patellae Micrognathia Syndrome, Microvillus Inclusion Disease, MID, Midsystolic-click-late systolic murmur syndrome, Miescher's Type I Syndrome, Mikulicz Syndrome, Mikulicz-Radecki Syndrome, Mikulicz-Sjogren Syndrome, Mild Autosomal Recessive, Mild Intermediate Maple Syrup Urine Disease, Mild Maple Syrup Urine Disease, Miller Syndrome, Miller-Dieker Syndrome, Miller-Fisher Syndrome, Mifroy Disease, Minkowski-Chauffard Syndrome, Minor Epilepsy, Minot-Von Willebrand Disease, Mirror-Image Dextrocardia, Mitochondrial Beta-Oxidation Disorders, Mitrochondrial and Cytosolic, Mitochondrial Cytopathy, Mitochondrial Cytopathy, Kearn-Sayre Type, Mitochondrial Encephalopathy, Mitochondrial Encephalo myopathy Lactic Acidosis and Strokelike Episodes, Mitochondrial myopathy, Mitochondrial myopathy Encephalopathy Lactic Acidosis Stroke-Like Episode, Mitochondrial PEPCK Deficiency, Mitral-valve prolapse, Mixed Apnea, Mixed Connective Tissue Disease, Mixed Hepatic Porphyria, Mixed Non-Fluent Aphasia, Mixed Sleep Apnea, Mixed Tonic and Clonic Torticollis, MJD, MKS, ML I, ML II, ML III, ML IV, ML Disorder Type I, ML Disorder Type II, ML Disorder Type III, ML Disorder Type IV, MLNS, MMR Syndrome, MND, MNGIE, MNS, Mobitz I, Mobitz II, Mobius Syndrome, Moebius Syndrome, Moersch-Woltmann Syndrome, Mohr Syndrome, Monilethrix, Monomodal Visual Amnesia, Mononeuritis Multiplex, Mononeuritis Peripheral, Mononeuropathy Peripheral, Monosomy 3p2, Monosomy 9p Partial, Monosomy 11q Partial, Monosomy 13q Partial, Monosomy 18q Syndrome, Monosomy X, Monostotic Fibrous Dysplasia, Morgagni-Turner-Albright Syndrome, Morphea, Morquio Disease, Morquio Syndrome, Morquio Syndrome A, Morquio Syndrome B, Morquio-Brailsford Syndrome, Morvan Disease, Mosaic Tetrasomy 9p, Motor Neuron Disease, Motor Neuron Syndrome, Motor Neurone Disease, Motoneuron Disease, Motoneurone Disease, Motor System Disease (Focal and Slow), Moya-moya Disease, Moyamoya Disease, MPS, MPS I, MPS I H, MPS 1 H/S Hurler/Scheie Syndrome, MPS I S Scheie Syndrome, MPS II, MPS IIA, MPS IIB, MPS II-AR Autosomal Recessive Hunter Syndrome, MPS II-XR, MPS II-XR Severe Autosomal Recessive, MPS III, MPS III A B C and D Sanfiloppo A, MPS IV, MPS IV A and B Morquio A, MPS V, MPS VI, MPS VI Severe Intermediate Mild Maroteaux-Lamy, MPS VII, MPS VII Sly Syndrome, MPS VIII, MPS Disorder, MPS Disorder I, MPS Disorder II, MPS Disorder III, MPS Disorder VI, MPS Disorder Type VII, MRS, MS, MSA, MSD, MSL, MSS, MSUD, MSUD, MSUD Type Ib, MSUD Type II, Mucocutaneous Lymph Node Syndrome, Mucolipidosis I, Mucolipidosis II, Mucolipidosis III, Mucolipidosis IV, Mucopolysaccharidosis, Mucopolysaccharidosis I-H, Mucopolysaccharidosis I-S, Mucopolysaccharidosis II, Mucopolysaccharidosis III, Mucopolysaccharidosis IV, Mucopolysaccharidosis VI, Mucopolysaccharidosis VII, Mucopolysaccharidosis Type I, Mucopolysaccharidosis Type II, Mucopolysaccharidosis Type III, Mucopolysaccharidosis Type VII, Mucosis, Mucosulfatidosis, Mucous Colitis, Mucoviscidosis, Mulibrey Dwarfism, Mulibrey Nanism Syndrome, Mullerian Duct Aplasia-Renal Aplasia-Cervicothoracic Somite Dysplasia, Mullerian Duct-Renal-Cervicothoracic-Upper Limb Defects, Mullerian Duct and Renal Agenesis with Upper Limb and Rib Anomalies, Mullerian-Renal-Cervicothoracic Somite Abnormalities, Multi-Infarct Dementia Binswanger's Type, Multicentric Castleman's Disease, Multifocal Eosinophilic Granuloma, Multiple Acyl-CoA Dehydrogenase Deficiency, Multiple Acyl-CoA Dehydrogenase Deficiency/Glutaric Aciduria Type II, Multiple Angiomas and Endochondromas, Multiple Carboxylase Deficiency, Multiple Cartilaginous Enchondroses, Multiple Cartilaginous Exostoses, Multiple Enchondromatosis, Multiple Endocrine Deficiency Syndrome Type II, Multiple Epiphyseal Dysplasia, Multiple Exostoses, Multiple Exostoses Syndrome, Multiple Familial Polyposis, Multiple Lentigines Syndrome, Multiple Myeloma, Multiple Neuritis of the Shoulder Girdle, Multiple Osteochondromatosis, Multiple Peripheral Neuritis, Multiple Polyposis of the Colon, Multiple Pterygium Syndrome, Multiple Sclerosis, Multiple Sulfatase Deficiency, Multiple Symmetric Lipomatosis, Multiple System Atrophy, Multisynostotic Osteodysgenesis, Multisynostotic Osteodysgenesis with Long Bone Fractures, Mulvihill-Smith Syndrome, MURCS Association, Murk Jansen Type Metaphyseal Chondrodysplasia, Muscle Carnitine Deficiency, Muscle Core Disease, Muscle Phosphofructokinase Deficiency, Muscular Central Core Disease, Muscular Dystrophy, Muscular Dystrophy Classic X-linked Recessive, Muscular Dystrophy Congenital With Central Nervous System Involvement, Muscular Dystrophy Congenital Progressive with Mental Retardation, Muscular Dystrophy Facioscapulohumeral, Muscular Rheumatism, Muscular Rigidity—Progressive Spasm, Musculoskeletal Pain Syndrome, Mutilating Acropathy, Mutism, mvp, MVP, MWS, Myasthenia Gravis, Myasthenia Gravis Pseudoparalytica, Myasthenic Syndrome of Lambert-Eaton, Myelinoclastic Diffuse Sclerosis, Myelomatosis, Myhre Syndrome, Myoclonic Astatic Petit Mal Epilepsy, Myoclonic Dystonia, Myoclonic Encephalopathy of Infants, Myoclonic Epilepsy, Myoclonic Epilepsy Hartung Type, Myoclonus Epilepsy Associated with Ragged Red Fibers, Myoclonic Epilepsy and Ragged-Red Fiber Disease, Myoclonic Progressive Familial Epilepsy, Myoclonic Progressive Familial Epilepsy, Myoclonic Seizure, Myoclonus, Myoclonus Epilepsy, Myoencephalopathy Ragged-Red Fiber Disease, Myofibromatosis, Myofibromatosis Congenital, Myogenic Facio-Scapulo-Peroneal Syndrome, Myoneurogastointestinal Disorder and Encephalopathy, Myopathic Arthrogryposis Multiplex Congenita, Myopathic Camitine Deficiency, Myopathy Central Fibrillar, myopathy Congenital Nonprogressive, myopathy Congenital Nonprogressive with Central Axis, myopathy with Deficiency of Carnitine Palmitoyltransferase, myopathy-Marinesco-Sjogren Syndrome, myopathy-Metabolic Carnitine Palmitoyltransderase Deficiency, myopathy Mitochondrial-Encephalopathy-Lactic Acidosis-Stroke, myopathy with Sarcoplasmic Bodies and Intermediate Filaments, Myophosphorylase Deficiency, Myositis Ossificans Progressiv, Myotonia Atrophica, Myotonia Congenita, Myotonia Congenita Intermittens, Myotonic Dystrophy, Myotonic myopathy Dwarfism Chondrodystrophy Ocular and Facial Anomalies, Myotubular myopathy, Myotubular myopathy X-linked, Myproic Acid, Myriachit (Observed in Siberia), Myxedema, N-Acetylglucosamine-1-Phosphotransferase Deficiency, N-Acetyl Glutamate Synthetase Deficiency, NADH-CoQ reductase deficiency, Naegeli Ectodermal Dysplasias, Nager Syndrome, Nager Acrofacial Dysostosis Syndrome, Nager Syndrome, NAGS Deficiency, Nail Dystrophy-Deafness Syndrome, Nail Dysgenesis and Hypodontia, Nail-Patella Syndrome, Nance-Horan Syndrome, Nanocephalic Dwarfism, Nanocephaly, Nanophthalmia, Narcolepsy, Narcoleptic syndrome, NARP, Nasal-fronto-faciodysplasia, Nasal Alar Hypoplasia Hypothyroidism Pancreatic Achylia Congenital Deafness, Nasomaxillary Hypoplasia, Nasu Lipodystrophy, NBIA1, ND, NDI, NDP, Necrotizing Encephalomyelopathy of Leigh's, Necrotizing Respiratory Ciranulomatosis, Neill-Dingwall Syndrome, Nelson Syndrome, Nemaline myopathy, Neonatal Adrenoleukodystrophy, Neonatal Adrenoleukodystrophy (NALD), Neonatal Adrenoleukodystrophy (ALD), Neonatal Autosomal Recessive Polycystic Kidney Disease, Neonatal Dwarfism, Neonatal Hepatitis, Neonatal Hypoglycemia, Neonatal Lactose Intolerance, Neonatal Lymphedema due to Exudative Enteropathy, Neonatal Progeroid Syndrome, Neonatal Pseudo-Hydrocephalic Progeroid Syndrome of Wiedemann-Rautenstrauch, Neoplastic Arachnoiditis, Nephroblastom, Nephrogenic Diabetes Insipidus, Nephronophthesis Familial Juvenile, Nephropathic Cystinosis, Nephropathy-Pseudohermaphroditism-Wilms Tumor, Nephrosis-Microcephaly Syndrome, Nephrosis-Neuronal Dysmigration Syndrome, Nephrotic-Glycosuric-Dwarfism-Rickets-Hypophosphatemic Syndrome, Netherton Disease, Netherton Syndrome, Netherton Syndrome Ichthyosis, Nettleship Falls Syndrome (X-Linked), Neu-Laxova Syndrome, Neuhauser Syndrome, Neural-tube defects, Neuralgic Amyotrophy, Neuraminidase Deficiency, Neuraocutaneous melanosis, Neurinoma of the Acoustic Nerve, Neurinoma, Neuroacanthocytosis, Neuroaxonal Dystrophy Schindler Type, Neurodegeneration with brain iron accumulation type 1 (NBIA1), Neurofibroma of the Acoustic Nerve, Neurogenic Arthrogryposis Multiplex Congenita, Neuromyelitis Optica, Neuromyotonia, Neuromyotonia, Focal, Neuromyotonia, Generalized, Familial, Neuromyotonia, Generalized, Sporadic, Neuronal Axonal Dystrophy Schindler Type, Neuronal Ceroid Lipofuscinosis Adult Type, Neuronal Ceroid Lipofuscinosis Juvenile Type, Neuronal Ceroid Lipofuscinosis Type 1, Neuronopathic Acute Gaucher Disease, Neuropathic Amyloidosis, Neuropathic Beriberi, Neuropathy Ataxia and Retinitis Pigmentosa, Neuropathy of Brachialpelxus Syndrome, Neuropathy Hereditary Sensory Type I, Neuropathy Hereditary Sensory Type II, Neutral Lipid Storage Disease, Nevii, Nevoid Basal Cell Carcinoma Syndrome, Nevus, Nevus Cavernosus, Nevus Comedonicus, Nevus Depigmentosus, Nevus Sebaceous of Jadassohn, Nezelof's Syndrome, Nezelof's Thymic Aplasia, Nezelof Type Severe Combined Immunodeficiency, NF, NF1, NF2, NF-1, NF-2, NHS, Niemann Pick Disease, Nieman Pick disease Type A (acute neuronopathic form), Nieman Pick disease Type B, Nieman Pick Disease Type C (chronic neuronopathic form), Nieman Pick disease Type D (Nova Scotia variant), Nieman Pick disease Type E, Nieman Pick disease Type F (sea-blue histiocyte disease), Night Blindness, Nigrospinodentatal Degeneration, Niikawakuroki Syndrome, NLS, NM, Noack Syndrome Type I, Nocturnal Myoclonus Hereditary Essential Myoclonus, Nodular Cornea Degeneration, Non-Bullous CIE, Non-Bullous Congenital Ichthyosiform Erythroderma, Non-Communicating Hydrocephalus, Non-Deletion Type Alpha-Thalassemia/Mental Retardation syndrome, Non-Ketonic Hyperglycinemia Type I (NKHI), Non-Ketotic Hyperglycinemia, Non-Lipid Reticuloendotheliosis, Non-Neuronopathic Chronic Adult Gaucher Disease, Non-Scarring Epidermolysis Bullosa, Nonarteriosclerotic Cerebral Calcifications, Nonarticular Rheumatism, Noncerebral, Juvenile Gaucher Disease, Nondiabetic Glycosuria, Nonischemic Cardio myopathy, Nonketotic Hypoglycemia and Carnitine Deficiency due to MCAD Deficiency, Nonketotic Hypoglycemia Caused by Deficiency of Acyl-CoA Dehydrogenase, Nonketotic Glycinemia, Nonne's Syndrome, Nonne-Milroy-Meige Syndrome, Nonopalescent Opalescent Dentine, Nonpuerperal Galactorrhea-Amenorrhea, Nonsecretory Myeloma, Nonspherocytic Hemolytic Anemia, Nontropical Sprue, Noonan Syndrome, Norepinephrine, Normal Pressure Hydrocephalus, Norman-Roberts Syndrome, Norrbottriian Gaucher Disease, Norrie Disease, Norwegian Type Hereditary Cholestasis, NPD, NPS, NS, NSA, Nuchal Dystonia Dementia Syndrome, Nutritional Neuropathy, Nyhan Syndrome, OAV Spectrum, Obstructive Apnea, Obstructive Hydrocephalus, Obstructive Sleep Apnea, OCC Syndrome, Occlusive Thromboaortopathy, OCCS, Occult Intracranial Vascular Malformations, Occult Spinal Dysraphism Sequence, Ochoa Syndrome, Ochronosis, Ochronotic Arthritis, OCR, OCRL, Octocephaly, Ocular Albinism, Ocular Herpes, Ocular Myasthenia Gravis, Oculo-Auriculo-Vertebral Dysplasia, Oculo-Auriculo-Vertebral Spectrum, Oculo-Bucco-Genital Syndrome, Oculocerebral Syndrome with Hypopigmentation, Oculocerebrocutaneous. Syndrome, Oculo-Cerebro-Renal, Oculocerebrorenal Dystrophy, Oculocerebrorenal Syndrome, Oculocraniosomatic Syndrome (obsolete), Oculocutaneous Albinism, Oculocutaneous Albinism Chediak-Higashi Type, Oculo-Dento-Digital Dysplasia, Oculodentodigital Syndrome, Oculo-Dento-Osseous Dysplasia, Oculo Gastrointestinal Muscular Dystrophy, Oculo Gastrointestinal Muscular Dystrophy, Oculomandibulodyscephaly with hypotrichosis, Oculomandibulofacial Syndrome, Oculomotor with Congenital Contractures and Muscle Atrophy, Oculosympathetic Palsy, ODD Syndrome, ODOD, Odontogenic Tumor, Odontotrichomelic Syndrome, OFD, OFD Syndrome, Ohio Type Amyloidosis (Type VII), OI, OI Congenita, OI Tarda, Oldfield Syndrome, Oligohydramnios Sequence, Oligophrenia Microphthalmos, Oligophrenic Polydystrophy, Olivopontocerebellar Atrophy, Olivopontocerebellar Atrophy with Dementia and Extrapyramidal Signs, Olivopontocerebellar Atrophy with Retinal Degeneration, Olivopontocerebellar Atrophy I, Olivopontocerebellar Atrophy II, Olivopontocerebellar Atrophy III, Olivopontocerebellar Atrophy IV, Olivopontocerebellar Atrophy V, Ollier Disease, Ollier Osteochondromatosis, Omphalocele-Visceromegaly-Macroglossia Syndrome, Ondine's Curse, Onion-Bulb Neuropathy, Onion Bulb Polyneuropathy, Onychoosteodysplasia, Onychotrichodysplasia with Neutropenia, OPCA, OPCA I, OPCA II, OPCA III, OPCA IV, OPCA V, OPD Syndrome, OPD Syndrome Type I, OPD Syndrome Type II, OPD I Syndrome, OPD II Syndrome, Ophthalmoarthropathy, Ophthalmoplegia-Intestinal Pseudoobstruction, Ophthalmoplegia, Pigmentary Degeneration of the Retina and Cadio myopathy, Ophthalmoplegia Plus Syndrome, Ophthalmoplegia Syndrome, Opitz BBB Syndrome, Opitz BBB/G Compound Syndrome, Opitz BBBG Syndrome, Opitz-Frias Syndrome, Opitz G Syndrome, Opitz G/BBB Syndrome, Opitz Hypertelorism-Hypospadias Syndrome, Opitz-Kaveggia Syndrome, Opitz Oculogenitolaryngeal Syndrome, Opitz Trigonocephaly Syndrome, Opitz Syndrome, Opsoclonus, Opsoclonus-Myoclonus, Opthamoneuromyelitis, Optic Atrophy Polyneuropathy and Deafness, Optic Neuroencephalomyelopathy, Optic Neuromyelitis, Opticomyelitis, Optochiasmatic Arachnoiditis, Oral-Facial Clefts, Oral-facial Dyskinesia, Oral Facial Dystonia, Oral-Facial-Digital Syndrome, Oral-Facial-Digital Syndrome Type I, Oral-Facial-Digital Syndrome I, Oral-Facial-Digital Syndrome II, Oral-Facial-Digital Syndrome III, Oral-Facial-Digital Syndrome IV, Orbital Cyst with Cerebral and Focal Dermal Malformations, Ornithine Carbamyl Transferase Deficiency, Ornithine Transcarbamylase Deficiency, Orocraniodigital Syndrome, Orofaciodigital Syndrome, Oromandibular Dystonia, Orthostatic Hypotension, Osler-Weber-Rendu disease, Osseous-Oculo-Dento Dysplasia, Osseous-Oculo-Dento D)ysplasia, Osteitis deformans, Osteochondrodystrophy Deformans, Osteochondroplasia, Osteodysplasty of Melnick and Needles, Osteogenesis Imperfect, Osteogenesis Imperfecta, Osteogenesis Imperfecta Congenita, Osteogenesis Imperfecta Tarda, Osteohypertrophic Nevus Flammeus, Osteopathia Hyperostotica Scleroticans Multiplex Infantalis, Osteopathia Hyperostotica Scleroticans Multiplex Infantalis, Osteopathyrosis, Osteopetrosis, Osteopetrosis Autosomal Dominant Adult Type, Osteopetrosis Autosomal Recessive Malignant Infantile Type, Osteopetrosis Mild Autosomal Recessive Intermediate Typ, Osteosclerosis Fragilis Generalisata, Osteosclerotic Myeloma, Ostium Primum Defect (endocardial cushion defects included), Ostium Secundum Defect, OTC Deficiency, Oto Palato Digital Syndrome, Oto-Palato-Digital Syndrome Type I, Oto-Palatal-Digital Syndrome Type II, Otodental Dysplasia, Otopalatodigital Syndrome, Otopalataldigital Syndrome Type II, Oudtshoorn Skin, Ovarian Dwarfism Turner Type, Ovary Aplasia Turner Type, OWR, Oxalosis, Oxidase deficiency, Oxycephaly, Oxycephaly-Acrocephaly, P-V, PA, PAC, Pachyonychia Ichtyosiforme, Pachyonychia Congenita with Natal Teeth, Pachyonychia Congenita, Pachyonychia Congenita Keratosis Disseminata Circumscripta (follicularis), Pachyonychia Congenita Jadassohn-Lewandowsky Type, PAF with MSA, Paget's Disease, Paget's Disease of Bone, Paget's Disease of the Breast, Paget's Disease of the Nipple, Paget's Disease of the Nipple and Areola, Pagon Syndrome, Painful Ophthalmoplegia, PAIS, Palatal Myoclonus, Palato-Oto-Digital Syndrome, Palatal-Oto-Digital Syndrome Type I, Palatal-Oto-Digital Syndrome Type II, Pallister Syndrome, Pallister-Hall Syndrome, Pallister-Killian Mosaic Syndrome, Pallister Mosaic Aneuploidy, Pallister Mosaic Syndrome, Pallister Mosaic Syndrome Tetrasomy 12p, Pallister-W Syndrome, Palmoplantar Hyperkeratosis and Alopecia, Palsy, Pancreatic Fibrosis, Pancreatic Insufficiency and Bone Marrow Dysfunction, Pancreatic Ulcerogenic Tumor Syndrome, Panmyelophthisis, Panmyelopathy, Pantothenate kinase associated neurodegeneration (PKAN), Papillon-Lefevre Syndrome, Papillotonic Psuedotabes, Paralysis Periodica Paramyotonica, Paralytic Beriberi, Paralytic Brachial Neuritis, Paramedian Lower Lip Pits-Popliteal Pyerygium Syndrome, Paramedian Diencephalic Syndrome, Paramyeloidosis, Paramyoclonus Multiple, Paramyotonia Congenita, Paramyotonia Congenita of Von Eulenburg, Parkinson's disease, Paroxysmal Atrial Tachycardia, Paroxysmal Cold Hemoglobinuria, Paroxysmal Dystonia, Paroxysmal Dystonia Choreathetosis, Paroxysmal Kinesigenic Dystonia, Paroxysmal Nocturnal Hemoglobinuria, Paroxysmal Normal Hemoglobinuria, Paroxysmal Sleep, Parrot Syndrome, Parry Disease, Parry-Romberg Syndrome, Parsonage-Turner Syndrome, Partial Androgen Insensitivity Syndrome, Partial Deletion of the Short Arm of Chromosome 4, Partial Deletion of the Short Arm of Chromosome 5, Partial Deletion of Short Arm of Chromosome 9, Partial Duplication 3q Syndrome, Partial Duplication 15q Syndrome, Partial Facial Palsy With Urinary Abnormalities, Partial Gigantism of Hands and Feet-Nevi-Hemihypertrophy-Macrocephaly, Partial Lipodystrophy, Partial Monosomy of Long Arm of Chromosome 11, Partial Monosomy of the Long Arm of Chromosome 13, Partial Spinal Sensory Syndrome, Partial Trisomy 11q, Partington Syndrome, PAT, Patent Ductus Arteriosus, Pathological Myoclonus, Pauciarticular-Onset Juvenile Arthritis, Paulitis, PBC, PBS, PC Deficiency, PC Deficiency Group A, PC Deficiency Group B, PC, Eulenburg Disease, PCC Deficiency, ′PCH, PCLD, PCT, PD, PDA, PDH Deficiency, Pearson Syndrome Pyruvate Carboxylase Deficiency, Pediatric Obstructive Sleep Apnea, Peeling Skin Syndrome, Pelizaeus-Merzbacher Disease, Pelizaeus-Merzbacher Brain Sclerosis, Peflagra-Cerebellar Ataxia-Renal Aminoaciduria Syndrome, Pelvic Pain Syndrome, Pemphigus Vulgaris, Pena Shokeir II Syndrome, Pena Shokeir Syndrome Type II, Penile Fibromatosis, Penile Fibrosis, Penile Induration, Penta X Syndrome, Pentalogy of Cantrell, Pentalogy Syndrome, Pentasomy X, PEPCK Deficiency, Pepper Syndrome, Perheentupa Syndrome, Periarticular Fibrositis, Pericardial Constriction with Growth Failure, Pericollagen Amyloidosis, Perinatal Polycystic Kidney Diseases, Perineal Anus, Periodic Amyloid Syndrome, Periodic Peritonitis Syndrome, Periodic Somnolence and Morbid Hunger, Periodic Syndrome, Peripheral Cystoid Degeneration of the Retina, Peripheral Dysostosis-Nasal Hypoplasia-Mental Retardation, Peripheral Neuritis, Peripheral Neuropathy, Peritoneopericardial Diaphragmatic Hernia, Pernicious Anemia, Peromelia with Micrognathia, Peroneal Muscular Atrophy, Peroneal Nerve Palsy, Peroutka Sneeze, Peroxisomal Acyl-CoA Oxidase, Peroxisomal Beta-Oxidation Disorders, Peroxisomal Bifunctional Enzyme, Peroxisomal Thiolase, Peroxisomal Thiolase Deficiency, Persistent Truncus Arteriosus, Perthes Disease, Petit Mal Epilepsy, Petit Mal Variant, Peutz-Jeghers Syndrome, Peutz-Touraine Syndrome, Peyronie Disease, Pfeiffer, Pfeiffer Syndrome Type I, PGA I, PGA II, PGA III, PGK, PH Type I, PH Type I, Pharyngeal Pouch Syndrome, PHI) Short-Chain Acyl-CoA Dehydrogenase Deficiency, Phenylalanine Hydroxylase Deficiency, Phenylalaninemia, Phenylketonuria, Phenylpyruvic Oligophrenia, Phocomelia, Phocomelia Syndrome, Phosphoenolpyruvate Carboxykinase Deficiency, Phosphofructokinase Deficiency, Phosphoglycerate Kinase Deficiency, Phosphoglycerokinase, Phosphorylase 6 Kinase Deficiency, Phosphorylase Deficiency Glycogen Storage Disease, Phosphorylase Kinase Deficiency of Liver, Photic Sneeze Reflex, Photic Sneezing, Phototherapeutic keratectomy, PHS, Physicist John Dalton, Phytanic Acid Storage Disease, Pi Phenotype ZZ, PI, Pick Disease of the Brain, Pick's Disease, Pickwickian Syndrome, Pierre Robin Anomalad, Pierre Robin Complex, Pierre Robin Sequence, Pierre Robin Syndrome, Pierre Robin Syndrome with Hyperphalangy and Clinodactyly, Pierre-Marie's Disease, Pigmentary Degeneration of Globus Pallidus Substantia Nigra Red Nucleus, Pili Torti and Nerve Deafness, Pili Torti-Sensorineural Hearing Loss, Pituitary Dwarfism II, Pituitary Tumor after Adrenalectomy, Pityriasis Pilaris, Pityriasis Rubra Pilaris, PJS, PKAN, PKD, PKD1, PKD2, PKD3, PKU, PKU1, Plagiocephaly, Plasma Cell Myeloma, Plasma Cell Leukemia, Plasma Thromboplastin Component Deficiency, Plasma Transglutaminase Deficiency, Plastic Induration Corpora Cavernosa, Plastic Induration of the Penis, PLD, Plicated Tongue, PLS, PMD, Pneumorenal Syndrome, PNH, PNM, PNP Deficiency, POD, POH, Poikiloderma Atrophicans and Cataract, Poikiloderma Congenitale, Poland Anomaly, Poland Sequence, Poland Syndactyly, Poland Syndrome, Poliodystrophia Cerebri Progressiva, Polyarthritis Enterica, Polyarteritis Nodosa, Polyarticular-Onset Juvenile Arthritis Type I, Polyarticular-Onset Juvenile Arthritis Type II, Polyarticular-Onset Juvenile Arthritis Types I and II, Polychondritis, Polycystic Kidney Disease, Polycystic Kidney Disease Medullary Type, Polycystic Liver Disease, Polycystic Ovary Disease, Polycystic Renal Diseases, Polydactyly-Joubert Syndrome, Polydysplastic Epidermolysis Bullosa, Polydystrophia Oligophrenia, Polydystrophic Dwarfism, Polyglandular Autoimmune Syndrome Type III, Polyglandular Autoimmune Syndrome Type II, Polyglandular Autoimmune Syndrome Type I, Polyglandular Autoimmune Syndrome Type II, Polyglandular Deficiency Syndrome Type II, Polyglandular Syndromes, Polymorphic Macula Lutea Degeneration, Polymorphic Macular Degeneration, Polymorphism of Platelet Grlycoprotien Ib, Polymorphous Corneal Dystrophy Hereditary, Polymyalgia Rheumatica, Polymyositis and Dermatomyositis, Primary Agammaglobulinemia, Polyneuritis Peripheral, Polyneuropathy-Deafness-Optic Atrophy, Polyneuropathy Peripheral, Polyneuropathy and Polyradiculoneuropathy, Polyostotic Fibrous Dysplasia, Polyostotic Sclerosing Histiocytosis, Polyposis Familial, Polyposis Gardner Type, Polyposis Hamartomatous Intestinal, Polyposis-Osteomatosis-Epidermoid Cyst Syndrome, Polyposis Skin Pigmentation Alopecia and Fingernail Changes, Polyps and Spots Syndrome, Polyserositis Recurrent, Polysomy Y, Polysyndactyly with Peculiar Skull Shape, Polysyndactyly-Dysmorphic Craniofacies Greig Type, Pompe Disease, Pompe Disease, Popliteal Pterygium Syndrome, Porcupine Man, Porencephaly, Porencephaly, Porphobilinogen deaminase (PBG-D), Porphyria, Porphyria Acute Intermittent, Porphyria ALA-D, Porphyria Cutanea Tarda, Porphyria Cutanea Tarda Hereditaria, Porphyria Cutanea Tarda Symptomatica, Porphyria Hepatica Variegate, Porphyria Swedish Type, Porphyria Variegate, Porphyriam Acute Intermittent, Porphyrins, Porrigo Decalvans, Port Wine Stains, Portuguese Type Amyloidosis, Post-Infective Polyneuritis, Postanoxic Intention Myoclonus, Postaxial Acrofacial Dysostosis, Postaxial Polydactyly, Postencephalitic Intention Myoclonus, Posterior Corneal Dystrophy Hereditary, Posterior Thalamic Syndrome, Postmyelographic Arachnoiditis, Postnatal Cerebral Palsy, Postoperative Cholestasis, Postpartum Galactorrhea-Amenorrhea Syndrome, Postpartum Hypopituitarism, Postpartum Panhypopituitary Syndrome, Postpartum Panhypopituitarism, Postpartum Pituitary Necrosis, Postural Hypotension, Potassium-Losing Nephritis, Potassium Loss Syndrome, Potter Type I Infantile Polycystic Kidney Diseases, Potter Type III Polycystic Kidney Disease, PPH, PPS, Prader-Willi Syndrome, Prader-Labhart-Willi Fancone Syndrome, Prealbumin Tyr-77 Amyloidosis, Preexcitation Syndrome, Pregnenolone Deficiency, Premature Atrial Contractions, Premature Senility Syndrome, Premature Supraventricular Contractions, Premature Ventricular Complexes, Prenatal or Connatal Neuroaxonal Dystrophy, Presenile Dementia, Presenile Macula Lutea Retinae Degeneration, Primary Adrenal Insufficiency, Primary Agammaglobulinemias, Primary Aldosteronism, Primary Alveolar Hypoventilation, Primary Amyloidosis, Primary Anemia, Primary Beriberi, Primary Biliary, Primary Biliary Cirrhosis, Primary Brown Syndrome, Primary Carnitine Deficiency, Primary Central Hypoventilation Syndrome, Primary Ciliary Dyskinesia Kartagener Type, Primary Cutaneous Amyloidosis, Primary Dystonia, Primary Failure Adrenocortical Insufficiency, Primary Familial Hypoplasia of the Maxilla, Primary Hemochromatosis, Primary Hyperhidrosis, Primary Hyperoxaluria [Type I], Primary Hyperoxaluria Type 1 (PH1), Primary Hyperoxaluria Type 1, Primary Hyperoxaluria Type II, Primary Hyperoxaluria Type III, Primary Hypogonadism, Primary Intestinal Lymphangiectasia, Primary Lateral Sclerosis, Primary Nonhereditary Amyloidosis, Primary Obliterative Pulmonary Vascular Disease, Primary Progressive Multiple Sclerosis, Primary Pulmonary Hypertension, Primary Reading Disability, Primary Renal Glycosuria, Primary Sclerosing Cholangitis, Primary Thrombocythemia, Primary Tumors of Central Nervous System, Primary Visual Agnosia, Proctocolitis Idiopathic, Proctocolitis Idiopathic, Progeria of Adulthood, Progeria of Childhood, Progeroid Nanism, Progeriod Short Stature with Pigmented Nevi, Progeroid Syndrome of De Barsy, Progressive Autonomic Failure with Multiple System Atrophy, Progressive Bulbar Palsy, Progressive Bulbar Palsy Included, Progressive Cardiomyopathic Lentiginosis, Progressive Cerebellar Ataxia Familial, Progressive Cerebral Poliodystrophy, Progressive Choroidal Atrophy, Progressive Diaphyseal Pysplasia, Progressive Facial Hemiatrophy, Progressive Familial Myoclonic Epilepsy, Progressive Hemifacial Atrophy, Progressive Hypoerythemia, Progressive Infantile Poliodystrophy, Progressive Lenticular Degeneration, Progressive Lipodystrophy, Progressive Muscular Dystrophy of Childhood, Progressive Myoclonic Epilepsy, Progressive Osseous Heteroplasia, Progressive Pallid Degeneration Syndrome, Progressive Spinobulbar Muscular Atrophy, Progressive Supranuclear Palsy, Progressive Systemic Sclerosis, Progressive Tapetochoroidal Dystrophy, Proline Oxidase Deficiency, Propionic Acidemia, Propionic Acidemia Type I (PCCA Deficiency), Propionic Acidemia Type II (PCCB Deficiency), Propionyl CoA Carboxylase Deficiency, Protanomaly, Protanopia, Protein-Losing Enteropathy Secondary to Congestive Heart Failure, Proteus Syndrome, Proximal Deletion of 4q Included, PRP, PRS, Prune Belly Syndrome, PS, Pseudo-Hurler Polydystrophy, Pseudo-Polydystrophy, Pseudoacanthosis Nigricans, Pseudoachondroplasia, Pseudocholinesterase Deficiency, Pseudogout Familial, Pseudohemophilia, Pseudohermaphroditism, Pseudohermaphroditism-Nephron Disorder-Wilm's Tumor, Pseudohypertrophic Muscular Dystrophy, Pseudohypoparathyroidism, Pseudohypophosphatasia, Pseudopolydystrophy, Pseudothalidomide Syndrome, Pseudoxanthoma Elasticum, Psoriasis, Psorospermosis Follicularis, PSP, PSS, Psychomotor Convulsion, Psychomotor Epilepsy, Psychomotor Equivalent Epilepsy, PTC Deficiency, Pterygium, Pterygium Colli Syndrome, Pterygium Universale, Pterygolymphangiectasia, Pulmonary Atresia, Pulmonary Lymphangiomyomatosis, Pulmonary Stenosis, Pulmonic Stenosis-Ventricular Septal Defect, Pulp Stones, Pulpal Dysplasia, Pulseless Disease, Pure Alymphocytosis, Pure Cutaneous Histiocytosis, Purine Nucleoside Phosphorylase Deficiency, Purpura Hemorrhagica, Purtilo Syndrome, PXE, PXE Dominant Type, PXE Recessive Type, Pycnodysostosis, Pyknodysostosis, Pyknoepilepsy, Pyroglutamic Aciduria, Pyroglutamicaciduria, Pyrroline Carboxylate pehydrogenase Deficiency, Pyruvate Carboxylase Deficiency, Pyruvate Carboxylase Deficiency Group A, Pyruvate Carboxylase Deficiency Group B, Pyruvate Dehydrogenase Deficiency, Pyruvate Kinase Deficiency, q25-qter, q26 or q27-qter, q31 or 32-qter, QT Prolongation with Extracellular Hypohypocalcinemia, QT Prolongation without Congenital Deafness, QT Prolonged with Congenital Deafness, Quadriparesis of Cerebral Palsy, Quadriplegia of Cerebral Palsy, Quantal Squander, Quantal Squander, r4, r6, r14, r 18, r21, r22, Rachischisis Posterior, Radial Aplasia-Amegakaryocytic Thrombocytopenia, Radial Aplasia-Thrombocytopenia Syndrome, Radial Nerve Palsy, Radicular Neuropathy Sensory, Radicular Neuropathy Sensory Recessive, Radicular Dentin Dysplasia, Rapid-onset Dystonia-parkinsonism, Rapp-Hodgkin Syndrome, Rapp-Hodgkin (hypohidrotic) Ectodermal Dysplasia syndrome, Rapp-Hodgkin Hypohidrotic Ectodermal Dysplasias, Rare hereditary ataxia with polyneuritic changes and deafness caused by a defect in the enzyme phytanic acid hydroxylase, Rautenstrauch-Wiedemann Syndrome, Rautenstrauch-Wiedemann Type Neonatal Progeria, Raynaud's Phenomenon, RDP, Reactive Functional Hypoglycemia, Reactive Hypoglycemia Secondary to Mild Diabetes, Recessive Type Kenny-Caffe Syndrome, Recklin Recessive Type Myotonia Congenita, Recklinghausen Disease, Rectoperineal Fistula, Recurrent Vomiting, Reflex Neurovascular Dystrophy, Reflex Sympathetic Dystrophy Syndrome, Refractive Errors, Refractory Anemia, Refrigeration Palsy, Refsum Disease, Refsum's Disease, Regional Enteritis, Reid-Barlow's syndrome, Reifenstein Syndrome, Reiger Anomaly-Growth Retardation, Reiger Syndrome, Reimann Periodic Disease, Reimann's Syndrome, Reis-Bucklers Corneal Dystrophy, Reiter's Syndrome, Relapsing Guillain-Barre Syndrome, Relapsing-Remitting Multiple Sclerosis, Renal Agenesis, Renal Dysplasia-Blindness Hereditary, Renal Dysplasia-Retinal Aplasia Loken-Senior Type, Renal Glycosuria, Renal Glycosuria Type A, Renal Glycosuria Type B, Renal Glycosuria Type O, Renal-Oculocerebrodystrophy, Renal-Retinal Dysplasia with Medullary Cystic Disease, Renal-Retinal Dystrophy Familial, Renal-Retinal Syndrome, Rendu-Osler-Weber Syndrome, Respiratory Acidosis, Respiratory Chain Disorders, Respiratory Myoclonus, Restless Legs. Syndrome, Restrictive Cardio myopathy, Retention Hyperlipemia, Rethore Syndrome (obsolete), Reticular Dysgenesis, Retinal Aplastic-Cystic Kidneys-Joubert Syndrome, Retinal Cone Degeneration, Retinal Cone Dystrophy, Retinal Cone-Rod Dystrophy, Retinitis Pigmentosa, Retinitis Pigmentosa and Congenital Deafness, Retinoblastoma, Retinol Deficiency, Retinoschisis, Retinoschisis Juvenile, Retraction Syndrome, Retrobulbar Neuropathy, Retrolenticular Syndrome, Rett Syndrome, Reverse Coarction, Reye Syndrome, Reye's Syndrome, RGS, Rh Blood Factors, Rh Disease, Rh Factor Incompatibility, Rh Incompatibility, Rhesus Incompatibility, Rheumatic Fever, Rheumatoid Arthritis, Rheumatoid Myositis, Rhinosinusogenic Cerebral Arachnoiditis, Rhizomelic Chondrodysplasia Punctata (RCDP), Acatalasemia, Classical Refsum disease, RHS, Rhythmical Myoclonus, Rib Gap Defects with Micrognathia, Ribbing Disease (obsolete), Ribbing Disease, Richner-Hanhart Syndrome, Rieger Syndrome, Rieter's Syndrome, Right Ventricular Fibrosis, Riley-Day Syndrome, Riley-Smith syndrome, Ring Chromosome 14, Ring Chromosome 18, Ring 4, Ring 4 Chromosome, Ring 6, Ring 6 Chromosome, Ring 9, Ring 9 Chromosome R9, Ring 14, Ring 15, Ring 15 Chromosome (mosaic pattern), Ring 18, Ring Chromosome 18, Ring 21, Ring 21 Chromosome, Ring 22, Ring 22 Chromosome, Ritter Disease, Ritter-Lyell Syndrome, RLS, RMSS, Roberts SC-Phocomelia Syndrome, Roberts Syndrome, Roberts Tetraphocomelia Syndrome, Robertson's Ectodermal Dysplasias, Robin Anomalad, Robin Sequence, Robin Syndrome, Robinow Dwarfism, Robinow Syndrome, Robinow Syndrome Dominant Form, Robinow Syndrome Recessive Form, Rod myopathy, Roger Disease, Rokitansky's Disease, Romano-Ward Syndrome, Romberg Syndrome, Rootless Teeth, Rosenberg-Chutorian Syndrome, Rosewater Syndrome, Rosselli-Gulienatti Syndrome, Rothmund-Thomson Syndrome, Roussy-Levy Syndrome, RP, RS X-Linked, RS, RSDS, RSH Syndrome, RSS, RSTS, RTS, Rubella Congenital, Rubinstein Syndrome, Rubinstein-Taybi Syndrome, Rubinstein Taybi Broad Thumb-Hallux syndrome, Rufous Albinism, Ruhr's Syndrome, Russell's Diencephalic Cachexia, Russell's Syndrome, Russell Syndrome, Russell-Silver Dwarfism, Russell-Silver Syndrome, Russell-Silver Syndrome X-linked, Ruvalcaba-Myhre-Smith syndrome (RMSS), Ruvalcaba Syndrome, Ruvalcaba Type Osseous Dysplasia with Mental Retardation, Sacral Regression, Sacral Agenesis Congenital, SAE, Saethre-Chotzen Syndrome, Sakati, Sakati Syndrome, Sakati-Nyhan Syndrome, Salaam Spasms, Salivosudoriparous Syndrome, Salzman Nodular Corneal Dystrophy, Sandhoff Disease, Sanfilippo Syndrome, Sanfilippo Type A, Sanfilippo Type B, Santavuori Disease, Santavuori-Haltia Disease, Sarcoid of Boeck, Sarcoidosis, Sathre-chotzen, Saturday Night Palsy, SBMA, SC Phocomelia Syndrome, SC Syndrome, SCA 3, SCAD Deficiency, SCAD Deficiency Adult-Onset Localized, SCAD Deficiency Congenital Generalized, SCAD, SCADH -Deficiency, Scalded Skin Syndrome, Scalp Defect Congenital, Scaphocephaly, Scapula Elevata, Scapuloperoneal myopathy, Scapuloperoneal Muscular Dystrophy, Scapuloperoneal Syndrome Myopathic Type, Scarring Bullosa, SCHAD, Schaumann's Disease, Scheie Syndrome, Schereshevkii-Turner Syndrome, Schilder Disease, Schilder Encephalitis, Schilder's Disease, Schindler Disease Type I (Infantile Onset), Schindler Disease Infantile Onset, Schindler Disease, Schindler Disease Type II (Adult Onset), Schinzel Syndrome, Schinzel-Giedion Syndrome, Schinzel Acrocallosal Syndrome, Schinzel-Giedion Midface-Retraction Syndrome, Schizencephaly, Schmid Type Metaphyseal Chondrodysplasia, Schmid Metaphyseal Dysostosis, Schmid-Fraccaro Syndrome, Schmidt Syndrome, Schopf-Schultz-Passarge Syndrome, Schueller-Christian Disease, Schut-Haymaker Type, Schwartz-Jampel-Aberfeld Syndrome, Schwartz-Jampel Syndrome Types 1A and 1B, Schwartz-Jampel Syndrome, Schwartz-Jampel Syndrome Type 2, SCID, Scleroderma, Sclerosis Familial Progressive Systemic, Sclerosis Diffuse Familial Brain, Scott Craniodigital Syndrome With Mental Retardation, Scrotal Tongue, SCS, SD, SDS, SDYS, Seasonal Conjunctivitis, Sebaceous Nevus Syndrome, Sebaceous nevus, Seborrheic Keratosis, Seborrheic Warts, Seckel Syndrome, Seckel Type Dwarfism, Second Degree Congenital Heart Block, Secondary Amyloidosis, Secondary Blepharospasm, Secondary Non-tropical Sprue, Secondary Brown Syndrome, Secondary Beriberi, Secondary Generalized Amyloidosis, Secondary Dystonia, Secretory Component Deficiency, Secretory IgA Deficiency, SED Tarda, SED Congenital, SEDC, Segmental linear achromic nevus, Segmental Dystonia, Segmental Myoclonus, Seip Syndrome, Seitelberger Disease, Seizures, Selective Deficiency of IgG Subclasses, Selective Mutism, Selective Deficiency of IgG Subclass, Selective IgM Deficiency, Selective Mutism, Selective IgA Deficiency, Self-Healing Histiocytosis, Semilobar Holoprosencephaly, Seminiferous Tubule Dysgenesis, Senile Retinoschisis, Senile Warts, Senior-Loken Syndrome, Sensory Neuropathy Hereditary Type I, Sensory Neuropathy Hereditary Type II, Sensory Neuropathy Hereditary Type I, Sensory Radicular Neuropathy, Sensory Radicular Neuropathy Recessive, Septic Progressive Granulomatosis, Septo-Optic Dysplasia, Serous Circumscribed Meningitis, Serum Protease Inhibitor Deficiency, Serum Carnosinase Deficiency, Setleis Syndrome, Severe Combined Immunodeficiency, Severe Combined Immunodeficiency with Adenosine Deaminase Deficiency, Severe Combined Immunodeficiency (SCID), Sex Reversal, Sexual Infantilism, SGB Syndrome, Sheehan Syndrome, Shields Type Dentinogenesis Imperfecta, Shingles,varicella-zoster virus, Ship Beriberi, SHORT Syndrome, Short Arm 18 Deletion Syndrome, Short Chain Acyl CoA Dehydrogenase Deficiency, Short Chain Acyl-CoA Dehydrogenase (SCAD) Deficiency, Short Stature and Facial Telangiectasis, Short Stature Facial/Skeletal Anomalies-Retardation-Macrodontia, Short Stature-Hyperextensibility-Rieger Anomaly-Teething Delay, Short Stature-Onychodysplasia, Short Stature Telangiectatic Erythema of the Face, SHORT Syndrome, Shoshin Beriberi, Shoulder girdle syndrome, Shprintzen-Goldberg Syndrome, Shulman Syndrome, Shwachman-Bodian Syndrome, Shwachman-Diamond Syndrome, Shwachman Syndrome, Shwachman-Diamond-Oski Syndrome, Shwachmann Syndrome, Shy Drager Syndrome, Shy-Magee Syndrome, SI Deficiency, Sialidase Deficiency, Sialidosis Type I Juvenile, Sialidosis Type II Infantile, Sialidosis, Sialolipidosis, Sick Sinus Syndrome, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Sideroblastic Anemias, Sideroblastic Anemia, Sideroblastosis, SIDS, Siegel-Cattan-Mamou Syndrome, Siemens-Bloch type Pigmented Dermatosis, Siemens Syndrome, Sieweriing-Creutzfeldt Disease, Siewert Syndrome, Silver Syndrome, Silver-Russell Dwarfism, Silver-Russell Syndrome, Simmond's Disease, Simons Syndrome, Simplex Epidermolysis Bullosa, Simpson Dysmorphia Syndrome, Simpson-Golabi-Behmel Syndrome, Sinding-Larsen-Johansson Disease, Singleton-Merten Syndrome, Sinus Arrhythmia, Sinus Venosus, Sinus tachycardia, Sirenomelia Sequence, Sirenomelus, Situs Inversus Bronchiectasis and Sinusitis, SJA Syndrome, Sjogren Larsson Syndrome Ichthyosis, Sjogren Syndrome, Sjögren's Syndrome, SJS, Skeletal dysplasia, Skeletal Dysplasia Weismann Netter Stuhl Type, Skin Peeling Syndrome, Skin Neoplasms, Skull Asymmetry and Mild Retardation, Skull Asymmetry and Mild Syndactyly, SLE, Sleep Epilepsy, Sleep Apnea, SLO, Sly Syndrome, SMA, SMA Infantile Acute Form, SMA I, SMA III, SMA type I, SMA type II, SMA type III, SMA3, SMAX1, SMCR, Smith Lemli Opitz Syndrome, Smith Magenis Syndrome, Smith-Magenis Chromosome Region, Smith-McCort Dwarfism, Smith-Opitz-Inborn Syndrome, Smith Disease, Smoldering Myeloma, SMS, SNE, Sneezing From Light Exposure, Sodium valproate, Solitary Plasmacytoma of Bone, Sorsby Disease, Sotos Syndrome, Souques-Charcot Syndrome, South African Genetic Porphyria, Spasmodic Dysphonia, Spasmodic Torticollis, Spasmodic Wryneck, Spastic Cerebral Palsy, Spastic Colon, Spastic Dysphonia, Spastic Paraplegia, SPD Calcinosis, Specific Antibody Deficiency with Normal Immunoglobulins, Specific Reading Disability, SPH2, Spherocytic Anemia, Spherocytosis, Spherophakia-Brachymorphia Syndrome, Sphingomyelin Lipidosis, Sphingomyelinase Deficiency, Spider fingers, Spielmeyer-Vogt Disease, Spielmeyer-Vogt-Batten Syndrome, Spina Bifida, Spina Bifida Aperta, Spinal Arachnoiditis, Spinal Arteriovenous Malformation, Spinal Ataxia Hereditofamilial, Spinal and Bulbar Muscular Atrophy, Spinal Diffuse Idiopathic Skeletal Hyperostosis, Spinal DISH, Spinal Muscular Atrophy, Spinal Muscular Atrophy All Types, Spinal Muscular Atrophy Type ALS, Spinal Muscular Atrophy-Hypertrophy of the Calves, Spinal Muscular Atrophy Type I, Spinal Muscular Atrophy Type III, Spinal Muscular Atrophy type 3, Spinal Muscular Atrophy-Hypertrophy of the Calves, Spinal Ossifying Arachnoiditis, Spinal Stenosis, Spino Cerebellar Ataxia, Spinocerebellar Atrophy Type I, Spinocerebellar Ataxia Type I (SCAI), Spinocerebellar Ataxia Type II (SCAII), Spinocerebellar Ataxia Type III (SCAIII), Spinocerebellar Ataxia Type III (SCA 3), Spinocerebellar Ataxia Type IV (SCAIV), Spinocerebellar Ataxia Type V (SCAV), Spinocerebellar Ataxia Type VI (SCAVI), Spinocerebellar Ataxia Type VII (SCAVII), Spirochetal Jaundice, Splenic Agenesis Syndrome, Splenic Ptosis, Splenoptosis, Split Hand Deformity-Mandibulofacial Dysostosis, Split Hand Deformity, Spondyloarthritis, Spondylocostal Dysplasia—Type I, Spondyloepiphyseal Dysplasia Tarda, Spondylothoracic Dysplasia, Spondylotic Caudal Radiculopathy, Sponge Kidney, Spongioblastoma Multiforme, Spontaneous Hypoglycemia, Sprengel Deformity, Spring Ophthalmia, SRS, ST, Stale Fish Syndrome, Staphyloccal Scalded Skin Syndrome, Stargardt's Disease, Startle Disease, Status Epilepticus, Steele-Richardson-Olszewski Syndrome, Steely Hair Disease, Stein-Leventhal Syndrome, Steinert Disease, Stengel's Syndrome, Stengel-Batten-Mayou-Spielmeyer-Vogt-Stock Disease, Stenosing Cholangitis, Stenosis of the Lumbar Vertebral Canal, Stenosis, Steroid Sulfatase Deficiency, Stevanovic's Ectodermal Dysplasias, Stevens Johnson Syndrome, STGD, Stickler Syndrome, Stiff-Man Syndrome, Stiff Person Syndrome, Still's Disease, Stilling-Turk-Duane Syndrome, Stillis Disease, Stimulus-Sensitive Myoclonus, Stone Man Syndrome, Stone Man, Streeter Anomaly, Striatonigral Degeneration Autosomal Dominant Type, Striopallidodentate Calcinosis, Stroma, Descemet's Membrane, Stromal Corneal Dystrophy, Struma Lymphomatosa, Sturge-Kalischer-Weber Syndrome, Sturge Weber Syndrome, Sturge-Weber Phakomatosis, Subacute Necrotizing Encephalomyelopathy, Subacute Spongiform Encephalopathy, Subacute Necrotizing Encephalopathy, Subacute Sarcoidosis, Subacute Neuronopathic, Subaortic Stenosis, Subcortical Arteriosclerotic Encephalopathy, Subendocardial Sclerosis, Succinylcholine Sensitivity, Sucrase-Isomaltase Deficiency Congenital, Sucrose-Isomaltose Malabsorption Congenital, Sucrose Intolerance Congenital, Sudanophilic Leukodystrophy ADL, Sudanophilic Leukodystrophy Pelizaeus-Merzbacher Type, Sudanophilic Leukodystrophy Included, Sudden Infant Death Syndrome, Sudeck's Atrophy, Sugio-Kajii Syndrome, Summerskill Syndrome, Summit Acrocephalosyndactyly, Summitt's Acrocephalosyndactyly, Summitt Syndrome, Superior Oblique Tendon Sheath Syndrome, Suprarenal glands, Supravalvular Aortic Stenosis, Supraventricular tachycardia, Surdicardiac Syndrome, Surdocardiac Syndrome, SVT, Sweat Gland Abscess, Sweating Gustatory Syndrome, Sweet Syndrome, Swiss Cheese Cartilage Syndrome, Syndactylic Oxycephaly, Syndactyly Type I with Microcephaly and Mental Retardation, Syndromatic Hepatic Ductular Hypoplasia, Syringomyelia, Systemic Aleukemic Reticuloendotheliosis, Systemic Amyloidosis, Systemic Carnitine Deficiency, Systemic Elastorrhexis, Systemic Lupus Erythematosus, Systemic Mast Cell Disease, Systemic Mastocytosis, Systemic-Onset Juvenile Arhritis, Systemic Sclerosis, Systopic Spleen, T-Lymphocyte Deficiency, Tachyalimentation Hypoglycemia, Tachycardia, Takahara syndrome, Takayasu Disease, Takayasu Arteritis, Talipes Calcaneus, Talipes Equinovarus, Talipes Equinus, Talipes Varus, Talipes Valgus, Tandem Spinal Stenosis, Tangier Disease, Tapetoretinal Degeneration, TAR Syndrome, Tardive Dystonia, Tardive Muscular Dystrophy, Tardive Dyskinesia, Tardive Oral Dyskinesia, Tardive Dystonia, Tardy Ulnar Palsy, Target Cell Anemia, Tarsomegaly, Tarui Disease, TAS Midline Defects Included, TAS Midline Defect, Tay Sachs Sphingolipidosis, Tay Sachs Disease, Tay Syndrome Ichthyosis, Tay Sachs Sphingolipidosis, Tay Syndrome Ichthyosis, Taybi Syndrome Type I, Taybi Syndrome, TCD, TCOF1, TCS, TD, TDO Syndrome, TDO-I, TDO-II, TDO-III, Telangiectasis, Telecanthus with Associated Abnormalities, Telecanthus-Hypospadias Syndrome, Temporal Lobe Epilepsy, Temporal Arteritis/Giant Cell Arteritis, Temporal Arteritis, TEN, Tendon Sheath Adherence Superior Obliqu, Tension Myalgia, Terminal Deletion of 4q Included, Terrian Corneal Dystrophy, Teschler-Nicola/Killian Syndrome, Tethered Spinal Cord Syndrome, Tethered Cord Malformation Sequence, Tethered Cord Syndrome, Tethered Cervical Spinal Cord Syndrome, Tetrahydrobiopterin Deficiencies, Tetrahydrobiopterin Deficiencies, Tetralogy of Fallot, Tetraphocomelia-Thrombocytopenia Syndrome, Tetrasomy Short Arm of Chromosome 9, Tetrasomy 9p, Tetrasomy Short Arm of Chromosome 18, Thalamic Syndrome, Thalamic Pain Syndrome, Thalamic Hyperesthetic Anesthesia, Thalassemia Intermedia, Thalassemia Minor, Thalassemia Major, Thiamine Deficiency, Thiamine-Responsive Maple Syrup Ulrine Disease, Thin-Basement-Membrane Nephropathy, Thiolase deficiency, RCDP, Acyl-CoA dihydroxyacetonephqsphate acyltransferase, Third and Fourth Pharyngeal Pouch Syndrome, Third Degree Congenital (Complete) Heart Block, Thomsen Disease, Thoracic-Pelvic-Phalangeal Dystrophy, Thoracic Spinal Canal, Thoracoabdominal Syndrome, Thoracoabdominal Ectopia Cordis Syndrome, Three M Syndrome, Three-M Slender-Boned Nanism, Thrombasthenia of Glanzmann and Naegeli, Thrombocythemia Essential, Thrombocytopenia-Absent Radius Syndrome, Thrombocytopenia-Hemangioma Syndrome, Thrombocytopenia-Absent Radii Syndrome, Thrombophilia Hereditary Due to AT III, Thrombotic Thrombocytopenic Purpura, Thromboulcerative Colitis, Thymic Dysplasia with Normal Immunoglobulins, Thymic Agenesis, Thymic Aplasia DiGeorge Type, Thymic Hypoplasia Agammaglobulinemias Primary Included, Thymic Hypoplasia DiGeorge Type, Thymus Congenital Aplasia, Tic Douloureux, Tics, Tinel's syndrome, Tolosa Hunt Syndrome, Tonic Spasmodic Torticollis, Tonic Pupil Syndrome, Tooth and Nail Syndrome, Torch Infection, TORCH Syndrome, Torsion Dystonia, Torticollis, Total Lipodystrophy, Total anomalous pulmonary venous connection, Touraine's Aphthosis, Tourette Syndrome, Tourette's disorder, Townes-Brocks Syndrome, Townes Syndrome, Toxic Paralytic Anemia, Toxic Epidermal Necrolysis, Toxopachyosteose Diaphysaire Tibio-Peroniere, Toxopachyosteose, Toxoplasmosis Other Agents Rubella Cytomegalovirus Herpes Simplex, Tracheoesophageal Fistula with or without Esophageal Atresia, Tracheoesophageal Fistula, Transient neonatal myasthenia gravis, Transitional Atrioventricular Septal Defect, Transposition of the great arteries, Transtelephonic Monitoring, Transthyretin. Methionine-30 Amyloidosis (Type I), Trapezoidocephaly-Multiple Synostosis Syndrome, Treacher Collins Syndrome, Treacher Collins-Franceschetti Syndrome 1, Trevor Disease, Triatrial Heart, Tricho-Dento-Osseous Syndrome, Trichodento Osseous Syndrome, Trichopoliodystrophy, Trichorhinophalangeal Syndrome, Trichorhinophalangeal Syndrome, Tricuspid atresia, Triflnctional Protein Deficiency, Trigeminal Neuralgia, Triglyceride Storage Disease Impaired Long-Chain Fatty Acid Oxidation, Trigonitis, Trigonocephaly, Trigonocephaly Syndrome, Trigonocephaly “C” Syndrome, Trimethylaminuria, Triphalangeal Thumbs-Hypoplastic Distal Phalanges-Onychodystrophy, Triphalangeal Thumb Syndrome, Triple Symptom Complex of Behcet, Triple X Syndrome, Triplo X Syndrome, Triploid Syndrome, Triploidy, Triploidy Syndrome, Trismus-Pseudocamptodactyly Syndrome, Trisomy, Trisomy G Syndrome, Trisomy X, Trisomy 6q Partial, Trisomy 6q Syndrome Partial, Trisomy 9 Mosaic, Trisomy 9P Syndrome (Partial) Included, Trisomy 11q Partial, Trisomy 14 Mosaic, Trisomy 14 Mosaicism Syndrome, Trisomy 21 Syndrome, Trisomy 22 Mosaic, Trisomy 22 Mosaicism Syndrome, TRPS, TRPS1, TRPS2, TRPS3, True Hermaphroditism, Truncus arteriosus, Tryptophan Malabsorption, Tryptophan Pyrrolase Deficiency, TS, TTP, TnTS, Tuberous Sclerosis, Tubular Ectasia, Turcot Syndrome, Turner Syndrome, Turner-Kieser Syndrome, Turner Phenotype with Normal Chromosomes (Karyotype), Turner-Vamy Syndrome, Turricephaly, Twin-Twin Transfusion Syndrome, Twin-to-Twin Transfusion Syndrome, Type A, Type B, Type AB, Type O, Type I Diabetes, Type I Familial Incomplete Male, Type I Familial Incomplete Male Pseudohermaphroditism, Type I Gaucher Disease, Type I (PCCA Deficiency), Type I Tyrosinemia, Type II Gaucher Disease, Type II Histiocytosis, Type II (PCCB Deficiency), Type II Tyrosinnemia, Type IIA Distal Artirogryposis Multiplex Congenita, Type III Gaucher Disease, Type III Tyrosinemia, Type III Dentinogenesis Imperfecta, Typical Retinoschisis, Tyrosinase Negative Albinism (Type I), Tyrosinase Positive Albinism (Type II), Tyrosinemia type 1 acute form, Tyrosinemia type 1 chronic form, Tyrosinosis, UCE, Ulcerative Colitis, Ulcerative Colitis Chronic Non-Specific, Ulnar-Mammary Syndrome, Ulnar-Mammary Syndrome of Pallister, Ulnar Nerve Palsy, UMS, Unclassified FODs, Unconjugated Benign Bilirubinemiav, Underactivity of Parathyroid, Unilateral Ichthyosiform Erythroderma with Ipsilateral Malformations Limb, Unilateral Chondromatosis, Unilateral Defect of Pectoralis Muscle and Syndactyly of the Hand, Unilateral Hemidysplasia Type, Unilateral Megalencephaly, Unilateral Partial Lipodystrophy, Unilateral Renal Agenesis, Unstable Colon, Unverricht Disease, Unverricht-Lundborg Disease, Unverricht-Lundborg-Laf Disease, Unverricht Syndrome, Upper Limb—Cardiovascular Syndrome (Holt-Oram), Upper Motor Neuron Disease, Upper Airway Apnea, Urea Cycle Defects or Disorders, Urea Cycle Disorder Arginase Type, Urea Cycle Disorder Arginino Succinase Type, Urea Cycle Disorders Carbamyl Phosphate Synthetase Type, Urea Cycle Disorder Citrullinemia Type, Urea Cycle Disorders N-Acrtyl Glutamate Synthetase Typ, Urea Cycle Disorder OTC Type, Urethral Syndrome, Urethro-Oculo-Articular Syndrome, Uridine Diphosphate Glucuronosyltransferase Severe Def. Type I, Urinary Tract Defects, Urofacial Syndrome, Uroporphyrinogen HI cosynthase, Urticaria pigmentosa, Usher Syndrome, Usher Type I, Usher Type II, Usher Type III, Usher Type IV, Uterine Synechiae, Uoporphyrinogen I-synthase, Uveitis, Uveomeningitis Syndrome, V-CJD, VACTEL Association, VACTERL Association, VACTERL Syndrome, Valgus Calcaneus, Valine Transaminase Deficiency, Valinemia, Valproic Acid, Valproate acid exposure, Valproic acid exposure, Valproic acid, Van Buren's Disease, Van der Hoeve-Habertsma-Waardenburg-Ciauldi Syndrome, Variable Onset Immunoglobulin Deficiency Dysgammaglobulinemia, Variant Creutzfeldt-Jakob Disease (V-CJD), Varicella Embryopathy, Variegate Porphyria, Vascular Birthmarks, Vascular Dementia Binswanger's Type, Vascular Erectile Tumor, Vascular Hemophilia, Vascular Malformations, Vascular Malformations of the Brain, Vasculitis, Vasomotor Ataxia, Vasopressin-Resistant Diabetes Insipidus, Vasopressin-Sensitive Diabetes Insipidus, VATER Association, Vcf syndrome, Vcfs, Velocardiofacial Syndrome, VeloCardioFacial Syndrome, Venereal Arthritis, Venous Malformations, Ventricular Fibrillation, Ventricular Septal Defects, Congenital Ventricular Defects, Ventricular Septal Defect, Ventricular Tachycardia, Venual Malformations, VEOHD, Vermis Aplasia, Vermis Cerebellar Agenesis, Vernal Keratoconjunctivitis, Verruca, Vertebral Anal Tracheoesophageal Esophageal Radial, Vertebral Ankylosing Hyperostosis, Very Early Onset Huntington's Disease, Very Long Chain Acyl-CoA Dehydrogenase (VLCAD) Deficiency, Vestibular Schwannoma, Vestibular Schwannoma Neurofibromatosis, Vestibulocerebellar, Virchow's Oxycephaly, Visceral Xanthogranulomatosis, Visceral Xantho-Granulomatosis, Visceral myopathy-Extemal Ophthalmoplegia, Visceromegaly-Umbilical Hernia-Macroglossia Syndrome, Visual Amnesia, Vitamin A Deficiency, Vitamin B-1 Deficiency, Vitelline Macular Dystrophy, Vitiligo, Vitiligo Capitis, Vitreoretinal Dystrophy, VKC, VKH Syndrome, VLCAD, Vogt Syndrome, Vogt Cephalosyndactyly, Vogt Koyanagi Harada Syndrome, Von Bechterew-Strumpell Syndrome, Von Eulenburg Paramyotonia Congenita, Von Frey's Syndrome, Von Gierke Disease, Von Hippel-Lindau Syndrome, Von Mikulicz Syndrome, Von Recklinghausen Disease, Von Willebrandt Disease, VP, Vrolik Disease (Type II), VSD, Vulgaris Type Disorder of Cornification, Vulgaris Type Ichthyosis, W Syndrome, Waardenburg Syndrome, Waardenburg-Klein Syndrome, Waardenburg Syndrome Type I (WS1), Waardenburg Syndrome Type II (WS2), Waardenburg Syndrome Type IIA (WS2A), Waardenburg Syndrome Type IIB (WS2B), Waardenburg Syndrome Type III (WS3), Waardenburg Syndrome Type IV (WS4), Waelsch's Syndrome, WAGR Complex, WAGR Syndrome, Waldenstroem's Macroglobulinemia, Waldenstrom's Purpura, Waldenstrom's Syndrome, Waldmann Disease, Walker-Warburg Syndrome, Wandering Spleen, Warburg Syndrome, Warm Antibody Hemolytic Anemia, Warm Reacting Antibody Disease, Wartenberg Syndrome, WAS, Water on the Brain, Watson Syndrome, Watson-Alagille Syndrome, Waterhouse-Friderichsen syndrome, Waxy Disease, WBS, Weaver Syndrome, Weaver-Smith Syndrome, Weber-Cockayne Disease, Wegener's Granulomatosis, Weil Disease, Weil Syndrome, Weill-Marchesani, Weill-Marchesani Syndrome, Weill-Reyes Syndrome, Weismann-Netter-Stuhl Syndrome, Weissenbacher-Zweymuller Syndrome, Wells Syndrome, Wenckebach, Werdnig-Hoffinan Disease, Werdnig-Hoffman Paralysis, Weribof s Disease, Werner Syndrome, Wernicke's (C) I Syndrome, Wernicke's aphasia, Wernicke-Korsakoff Syndrome, West Syndrome, Wet Beriberi, WHCR, Whipple's Disease, Whipple Disease, Whistling face syndrome, Whistling Face-Windmill Vane Hand Syndrome, White-Darier Disease, Whitnall-Norman Syndrome, Whorled nevoid hypermelanosis, WHS, Wieacker Syndrome, Wieacher Syndrome, Wieacker-Wolff Syndrome, Wiedmann-Beckwith Syndrome, Wiedemann-Rautenstrauch Syndrome, Wildervanck Syndrome, Willebrand-Juergens Disease, Willi-Prader Syndrome, Williams Syndrome, Williams-Beuren Syndrome, Wilms' Tumor, Wilms' Tumor-Aniridia-Gonadoblastoma-Mental Retardation Syndrome, Wilms Tumor Aniridia Gonadoblastoma Mental Retardation, Wilms' Tumor-Aniridia-Cienitourinary Anomalies-Mental Retardation Syndrome, Wilms Tumor-Pseudohermaphroditism-Nephropathy, Wilms Tumor and Pseudohermaphroditism, Wilms Tumor-Pseuodohermaphroditism-Glomerulopathy, Wilson's Disease, Winchester Syndrome, Winchester-Grossman Syndrome, Wiskott-Aldrich Syndrome, Wiskott-Aldrich Type Immunodeficiency, Witkop Ectodermal Dysplasias, Witkop Tooth-Nail Syndrome, Wittmaack-Ekbom Syndrome, WM Syndrome, WMS, WNS, Wohlfart-Disease, Wohlfart-Kugelberg-Welander Disease, Wolf Syndrome, Wolf-Hirschhom Chromosome Region (WHCR), Wolf-Hirschhorn Syndrome, Wolff-Parkinson-White Syndrome, Wolfram Syndrome, Wolman Disease (Lysomal Acid Lypase Deficiency), Woody Guthrie's Disease, WPW Syndrome, Writer's Cramp, WS, WSS, WWS, Wyburn-Mason Syndrome, X-Linked Addison's Disease, X-linked Adrenoleukodystrophy (X-ALD), X-linked Adult Onset Spinobulbar Muscular Atrophy, X-linked Adult Spinal Muscular Atrophy, X-Lin~ked Agammaglobulinemia with Growth Hormone Deficiency, X-Linked Agammaglobulinemia, Lymphoproliferate X-Linked Syndrome, X-linked Cardio myopathy and Neutropenia, X-Linked Centronuclear myopathy, X-linked Copper Deficiency, X-linked Copper Malabsorption, X-Linked Dominant Conradi-Hunermann Syndrome, X-Linked Dominant Inheritance Agenesis of Corpus Callosum, X-Linked Dystonia-parkinsonism, X Linked Ichthyosis, X-Linked Infantile Agammaglobulinemia, X-Linked Infantile Nectrotizing Encephalopathy, X-linked Juvenile Retinoschisis, X-linked Lissencephaly, X-linked Lymphoproliferative Syndrome, X-linked Mental Retardation-Clasped Thumb Syndrome, X-Linked Mental Retardation with Hypotonia, X-linked Mental Retardation and Macroorchidism, X-Linked Progressive Combined Variable Immunodeficiency, X-Linked Recessive Conradi-Hunermann Syndrome, X-Linked Recessive Severe Combined Immunodeficiency, X-Linked Retinoschisis, X-linked Spondyloepiphyseal Dysplasia, Xanthine Oxidase Deficiency (Xanthinuria Deficiency, Hereditary), Xanthinuria Deficiency, Hereditary (Xanthine Oxidase Deficiency), Xanthogranulomatosis Generalized, Xanthoma Tuberosum, Xeroderma Pigmentosum, Xeroderma Pigmentosum Dominant Type, Xeroderma Pigmentosum Type A I XPA Classical Form, Xeroderma Pigmentosum Type B II XPB, Xeroderma Pigmentosum Type E V XPE, Xeroderma Pigmentosum Type C III XPC, Xeroderma Pigmentosum Type D IV XPD, Xeroderma Pigmentosum Type F VI XPF, Xeroderma Pigmentosum Type G VII XPG, Xeroderma Pigmentosum Variant Type XP-V, Xeroderma-Talipes-and Enamel Defect, Xerodermic Idiocy, Xerophthalmia, Xerotic Keratitis, XLP, XO Syndrome, XP, XX Male Syndrome, Sex Reversal, XXXXX Syndrome, XXY Syndrome, XYY Syndrome, XYY Chromosome Pattern, Yellow Mutant Albinism, Yellow Nail Syndrome, YKL, Young Female Arteritis, Yunis-Varon Syndrome, YY Syndrome, Z-E Syndrome, Z- and -Protease Inhibitor Deficiency, Zellweger Syndrome, Zellweger cerebro-hepato-renal syndrome, ZES, Ziehen-Oppenheim Disease (Torsion Dystonia), Zimmermann-Laband Syndrome, Zinc Deficiency Congenital, Zinsser-Cole-Engman Syndrome, ZLS, Zollinger-Ellison Syndrome.

As used herein a “cancer” refers to a group of diseases and disorders that are characterized by uncontrolled cellular growth (e.g. formation of tumor) without any differentiation of those cells into specialized and different cells. Cancers which can be treated using the methods of the present invention include, without being limited to, ABL1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem ulioma, Brain and CNS Tumours, Breast Cancer, CNS tumqurs, Carcinoid Tumours, Cervical Cancer, Childhood Brain Tumours, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dermatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumours, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumours, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumours, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, Wilms' Tumour.

As used herein, a “brain disease or disorder” refers to any disease or disorder of the brain which results in either impaired cognitive ability or abnormal pathology. Brain diseases and disorders which can be treated using the methods of the present invention, include without being limited to, Acute Disseminated Encephalomyelitis, Arteriovenous Malformations and Other Vascular Lesions of the Central Nervous System, Cavernous Malformation, Cerebral Atrophy, Corticobasal Degeneration, Encephalopathy, Fahr's Syndrome, Kuru Moyamoya Disease, Neuronal Migration Disorders, Progressive Multifocal Leukoencephalopathy, Pseudotumor Cerebri (Benign Intracranial Hypertension), Transmissible Spongiform Encephalopathies, Wernicke-Korsakoff Syndrome, Chordoma Craniopharyngioma Medulloblastoma Meningioma Pineal Tumors Pituitary Adenoma Primitive Neuroectodermal Tumors Schwannoma Vascular Tumors, astrocytoma, glioblastomas, metastatic brain tumors, amyotrophic lateral sclerosis (ALS), progressive muscular atrophy, postpolio syndrome, Adrenoleukodystrophy, Alexander Disease, Alpers' Disease, Canavan Disease, Dementia with Lewy Bodies, Friedreich's Ataxia, Spanish Friedreich's Ataxia, Hallervorden-Spatz Disease, Krabbe Disease, Leigh's Disease, Leukodystrophy, Monomelic Amyotrophy, Olivopontocerebellar Atrophy, Opsoclonus Myoclonus, Paraneoplastic Syndromes, Pelizaeus-Merzbacher Disease, Progressive Multifocal Leukoencephalopathy, Progressive Supranuclear Palsy, Spanish Ramsay Hunt Syndrome Type II, Shy-Drager Syndrome, Alzheimer's disease, amyotrophic lateral sclerosis, aphasia, attention deficit disorder with hyperactivity, back pain, Bell's palsy, brain cancer, brain diseases, carpal tunnel syndrome, cerebral palsy, Charcot-Marie-tooth disease, Creutzfeldt-Jakob disease, degenerative nerve diseases, dementia, dizziness and vertigo, dystonia, encephalitis, epilepsy, Guillain-Barre syndrome, head and brain injuries, headache and migraine, hydrocephalus, memory, meningitis, movement disorders, multiple sclerosis, myasthenia gravis, neural tube defects, neurofibromatosis, neurologic diseases (general), pain, paralysis, Parkinson's disease, peripheral nerve disorders, phenylketonuria, pituitary disorders, reflex sympathetic dystrophy, restless legs, Reye syndrome, seizures, shingles (herpes zoster), sleep disorders, spina bifida, spinal cord diseases and injuries, spinal cord injuries, stroke, thoracic outlet syndrome, tourette syndrome, tremor, tuberous sclerosis, and West Nile virus.

As used herein “inflammatory diseases and disorders” encompass those disease and disorders which result in a response of redness, swelling, pain, and a feeling of heat in certain areas that is meant to protect tissues affected by injury or disease. Inflammatory diseases which can be treated using the methods of the present invention, include, without being limited to, acne, angina, arthritis, aspiration pneumonia, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections.

The treatment of diseases and disorders associated with immune function are also contemplated by the methods of the present invention. Immunosuppression is a disorder or condition where the immune response is reduced or absent. The immune system protects the body from potentially harmful substances (antigens) such as microorganisms, toxins, cancer cells, and blood or tissues from another person. The immune response consists of general actions such as phagocytosis, where white blood cells engulf and destroy “foreign” material. It protects against specific antigens by producing antibodies (immunoglobulins), which are molecules that attach to a specific antigen and make destruction of the antigen more efficient. It also protects against specific antigens by producing lymphocytes (a group of white blood cells) that become specialized (sensitized). The sensitized lymphocytes “recognize” the foreign substance, and destroy it.

Immunity is, in part, a product of lymphoid tissue in the body that includes the thymus, lymph nodes, tonsils, parts of the spleen and gastrointestinal tract, and bone marrow. Lymphocytes (the specialized white blood cells that provide acquired immunity) are produced or mature in various lymphoid tissues. Lymphocytes are divided into two groups. T lymphocytes become the sensitized lymphocytes that directly attack (cellular immunity). B lymphocytes produce antibodies (huumoral immunity) that attach to the antigen and make phagocytes and body chemicals such as complement proteins much more efficient in the destruction of the antigen.

Immune system disorders occur when the immune response is inappropriate, excessive, or lacking. Immunodeficiency disorders occur when the immune system fails to fight tumors or invading substances. This causes persistent or recurrent infections, severe infections by organisms that are normally mild, incomplete recovery from illness or poor response to treatment, and an increased incidence of cancer and other tumors. Opportunistic infections are widespread infections by microorganisms that are usually controllable.

This deficiency may affect any part of the immune system. Most commonly, it involves decreased functioning of T or B lymphocytes (or both), or deficient antibody production. The causes include congenital/inherited defects and acquired immunodeficiency caused by a disease that affects the immune system.

Examples of congenital rimmunodeficiency disorders of antibody production (B lymphocyte abnormalities) include hypogammaglobulinemia (lack of one or more specific antibodies), which usually causes repeated mild respiratory infections, and agammaglobulinemia (lack of all or most antibody production), which results in frequent severe infections and is often fatal. Congenital disorders affecting the T lymphocytes may cause increased susceptibility to fungi, resulting in repeated Candida (yeast) infections. Inherited combined immunodeficiency affects both T lymphocytes and B lymphocytes. It is often fatal within the first year of life because there is no resistance to disease or infection.

People are said to be “immunosuppressed” when they experience immunodeficiency that is caused by. medications such as corticosteroids or other immunosuppressant medications. This is a desired part of treatment for disorders such as autoimmune disorders. It is used after organ transplantation to prevent transplant rejections.

Immunosuppression is also a common side effect of chemotherapy to treat many types of cancer because the chemotherapy often reduces the number of white blood cells available to fight infection.

Acquired immunodeficiency may be a complication of diseases such as HIV infection and AIDS (acquired immunodeficiency syndrome). Malnutrition, particularly with lack of protein, can cause acquired immunodeficiency. Many cancers can cause immunodeficiency.

Those who have had a splenectomy, or removal of the spleen, face a higher risk of infection from certain encapsulated bacteria, such as but not limited to Streptococcus pneumoniae, that the spleen would normally help fight.

Increasing age also reduces the effectiveness of the immune system. Immune system tissues (particularly lymphoid tissue such as the thymus) shrink with aging. There is also reduced lymphocyte number and activity with increasing age.

The present invention, therefore, is directed in part, to the treatment of immunosuppressed individuals who are suffering from, for example, without limitation, Ataxia-telangiectasia, DiGeorge syndrome, Chediak-Higashi syndrome, Job syndrome, Leukocyte adhesion defects, Panhypogammaglobulinemia, Bruton disease, Congenital agammaglobulinemia, Selective deficiency of IgA, Combined immunodeficiency disease, Wiscott-Aldrich syndrome, and Complement deficiencies.

As used herein, the term “infertility” refers to the inability to conceive an offspring. Disease and disorders associated with in infertility which can be treated using the methods of the present invention include, without being limited to, Varicocoele, Galactorrhoea-Hyperprolactinaemia, Cryptorchism (maldescended or ectopic testis), Gonadal dysgenesis, Young's syndrome, Klinefelter's syndrome, Germinal cell aplasia, Haemochromatosis, Kallmann syndrome, Myotonic dystrophy, 5-Alpha reductase deficiency, Cystic fibrosis, Kartagener's syndrome, Incomplete androgen insensitivity, Kennedy's disease, Galactorrhoea-Hyperprolactinaemia, Hypopituitarism, Epididymo-orchitis, Pituitary tumour, Amenorrhoea (Specific type of Female ininfertility), Haemosiderosis, Hypokalaemic distal renal tubular acidosis, Idiopathic premature ovarian failure, Dyspareunia, Coalactorrhoea-Hyperprolactinaemia, FSH receptor deficiency, Gonadal dysgenesis (female), Mullerian dysgenesis, Trisomy X, Turner's syndrome, Kallmann syndrome, Myotonic dystrophy, C21-hydroxylase deficiency, Galactosaemia, Testicular feminization syndrome, Malabsorption syndrome, Conn's syndrome, Cushing's syndrome, Diabetes mellitus type 2, Galactorrhoea-Hyperprolactinaemia, Hyperthyroidism, Hypopituitarism, Hypothyroidism, Sheehan's syndrome, Autoimmune adrenalitis, Systemic lupus erythematosus, Adrenal cortex tumours, Pituitary tumour, Prolactin secreting pituitary tumour, Benign neoplastic conditions, Cushing's disease, Malignant neoplastic conditions, Ovarian cancer, Polycystic ovary syndrome and Pelvic inflammatory disease.

An agent includes proteinaceous or non-proteinaceous molecules such as antibodies, natural products, chemical entities or nucleic acid molecules (including antisense molecules, sense molecules, ribozymes, ds-RNA molecules or DNA-targeting molecules).

An “effective amount” means an amount necessary at least partly to attain the desired immune response (e.g. against AGT-701, AGT-702, AGT-704, AGT-705, AGT-706, AGT-707, AGT-708, AGT-709 or AGT-710) or to delay the onset or inhibit progression or halt altogether the onset or progression of a particular condition.

In accordance with these methods, AGT-701, AGT-702, AGT-704, AGT-705, AGT-706, AGT-707, AGT-708, AGT-709 and/or AGT-710 or AGT-701, AGT-702, AGT-704, AGT-705, AGT-706, AGT-707, AGT-708, AGT-709 and/or AGT-710 or agents capable of modulating the expression or activity of said molecules may be co-administered with one or more other compounds or Other molecules. EBy “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of AGT-701, AGT-702, AGT-704, AGT-705, AGT-706, AGT-707, AGT-708, AGT-709 and/or AGT-710 or a derivative, homolog or analog thereof in the manufacture of a medicament for the treatment of a condition characterized by healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

In still yet another aspect, the present invention relates to the use of an agent capable of modulating the activity of AGT-701, AGT-702, AGT-704, AGT-705, AGT-706, AGT-707, AGT-708, AGT-709 and/or AGT-710 or a derivative, homolog, analog, chemical equivalent or mimetic thereof in the manufacture of a medicament for the treatment of a condition characterized by healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

A further aspect of the present invention relates to the use of AGT-701, AGT-702, AGT-704, AGT-705, AGT-706, AGT-707, AGT-708, AGT-709 and/or AGT-710 or derivative, homolog or analog thereof or AGT-701, AGT-702, AGT-704, AGT-705, AGT-706, AGT-707, AGT-708, AGT-709 and/or AGT-710 or derivative, homolog, analog, chemical equivalent or mimetic thereof in the manufacture of a medicament for the treatment of a condition characterized by obesity, anorexia, weight maintenance, diabetes and/or energy imbalance.

Still yet another aspect of the present invention relates to agents for use in modulating the expression of AGT-701, AGT-702, AGT-704, AGT-705, AGT-706, AGT-707, AGT-708, AGT-709 and/or AGT-710 or a derivative, hqmolog or analog thereof.

A further aspect relates to agents for use in modulating AGT-701, AGT-702, AGT-704, AGT-705, AGT-706, AGT-707, AGT-708, AGT-709 and/or AGT-710 activity or a derivative, homolog, analog, chemical equivalent or mimetic thereof.

Still another aspect of the present invention relates to AGT-701, AGT-702, AGT-704, AGT-705, AGT-706, AGT-707, AGT-708, AGT-709 and/or AGT-710 or derivative, homolog or analog thereof or AGT-701, AGT-702, AGT-704, AGT-705, AGT-706, AGT-707, AGT-708, AGT-709 and/or AGT-710 or derivative, homolog, analog, chemical equivalent or mimetic thereof for use in treating a condition characterized by one or more symptoms of healthy state, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

In a related aspect of the present invention, the mammal undergoing treatment may be a human or an animal in need of therapeutic or prophylactic treatment.

The terms “treating” and “treatment” as used herein refer to a reduction in the severity and/or frequency of symptoms associated with inter alia myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels, including any condition associated with varying levels of selenoproteins, elimination of symptoms and/or the underlying cause, prevention of the occurrence of symptoms of disease and/or the underlying cause and improvement or remediation of damage.

“Treating” a subject may involve prevention of the disorder or disease condition or adverse physiological event in a susceptible individual as well as treatment of a clinically symptomatic individual by inhibiting a disease or disorder. Generally, such conditions involve, weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems weakness, hypotonia, cramping, muscle pain, proximal renal tubular wasting resulting in loss of protein, magnesium, phosphorous, calcium and other electrolytes, cardiac conduction defects (heart blocks) and cardio myopathy, hypoglycaemia (low blood sugar) and liver failure, visual loss and blindness, hearing loss and deafness, diabetes and exocrine pancreatic failure (inability to make digestive enzymes), mitochondrial dysfunction, including failure to gain weight, short statue, fatigue and respiratory problems.

Accordingly, the present invention contemplates in one embodiment a composition comprising a modulator of AGT-701, AGT-702, AGT-704, AGT-705, AGT-706, AGT-707, AGT-708, AGT-709 and AGT-710 expression or AGT-701, AGT-702, AGT-704, AGT-705, AGT-706, AGT-707, AGT-708, AGT-709 and AGT-710 activity and one or more pharmaceutically acceptable carriers and/or diluents. In another embodiment, the composition comprises AGT-701, AGT-702, AGT-704, AGT-705, AGT-706, AGT-707, AGT-708, AGT-709 and AGT-710 or a derivative, hqmolog, analog or mimetic thereof and one or more pharmaceutically acceptable carriers and/or diluents. The compositions may also comprise leptin or modulations of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must.be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When AGT-701, AGT-702, AGT-704, AGT-705, AGT-706, AGT-707, AGT-708, AGT-709 and AGT-710 or AGT-701, AGT-702, AGT-704, AGT-705, AGT-706, AGT-707, AGT-708, AGT-709 and AGT-710 are suitably protected, they may be orally administered, for example, with an inert diluent or with an assimilable edible carrer, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of AGT-701, AGT-702, AGT-704, AGT-705, AGT-706, AGT-707, AGT-708, AGT-709 and AGT-710 or AGT-701, AGT-702, AGT-704, AGT-705, AGT-706, AGT-707, AGT-708, AGT-709 and AGT-710 will range from 0.01 ng/kg/body weight to above 10,000 mg/kg/body weight. Alternative amounts range from 0.1 ng/kg/body weight to above 1000 mg/kg/body weight. The active ingredients may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating AGT-701, AGT-702, AGT-704, AGT-705, AGT-706, AGT-707, AGT-708, AGT-709 and AGT-710 expression or AGT-701, AGT-702, AGT-704, AGT-705, AGT-706, AGT-707, AGT-708, AGT-709 and AGT-710 activity. The vector may, for example, be a viral vector.

Still another aspect of the present invention is directed to antibodies to AGT-701, AGT-702, AGT-704, AGT-705, AGT-706, AGT-707, AGT-708, AGT-709 and AGT-710 and their derivatives and homologs insofar as AGT-701, AGT-702, AGT-704, AGT-705, AGT-706, AGT-707, AGT-708, AGT-709 and AGT-710 are proteins. Such antibodies may be monoclonal or polyclonal and may be selected from naturally occurring antibodies to AGT-701, AGT-702, AGT-704, AGT-705, AGT-706, AGT-707, AGT-708, AGT-709 and AGT-710 or may be specifically raised to AGT-701, AGT-702, AGT-704, AGT-705, AGT-706, AGT-707, AGT-708, AGT-709 and AGT-710 or derivatives or homologs thereof. In the case of the latter, AGT-701, AGT-702, AGT-704, AGT-705, AGT-706, AGT-707, AGT-708, AGT-709 and AGT-710 or their derivatives or homologs may first need to be associated with a carrier molecule. The antibodies and/or recombinant AGT-701, AGT-702, AGT-704, AGT-705, AGT-706, AGT-707, AGT-708, AGT-709 and AGT-710 or their derivatives of the present invention are particularly useful as therapeutic or diagnostic agents. An antibody “to” a molecule includes an antibody specific for said molecule.

AGT-701, AGT-702, AGT-704, AGT-705, AGT-706, AGT-707, AGT-708, AGT-709 and AGT-710 and their derivatives can be used to screen for naturally occurring antibodies to AGT-701, AGT-702, AGT-704, AGT-705, AGT-706, AGT-707, AGT-708, AGT-709 and AGT-710 which may occur in certain autoimmune diseases. Alternatively, specific antibodies can be used to screen for AGT-701, AGT-702, AGT-704, AGT-705, AGT-706, AGT-707, AGT-708, AGT-709 and AGT-710. Techniques for such assays are well known in the art and include, for example, sandwich assays and ELISA.

Antibodies to AGT-701, AGT-702, AGT-704, AGT-705, AGT-706, AGT-707, AGT-708, AGT-709 and AGT-710 of the present invention may be monoclonal or polyclonal and may be selected from naturally occurring antibodies to the AGT-701, AGT-702, AGT-704, AGT-705, AGT-706, AGT-707, AGT-708, AGT-709 and AGT-710 or may be specifically raised to these gene products. In the case of the latter, the AGT-701, AGT-702, AGT-704, AGT-705, AGT-706, AGT-707, AGT-708, AGT-709 and AGT-710 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying AGT-701, AGT-702, AGT-704, AGT-705, AGT-706, AGT-707, AGT-708, AGT-709 and AGT-710.

For example, specific antibodies can be used to screen for AGT-701, AGT-702, AGT-704, AGT-705, AGT-706, AGT-707, AGT-708, AGT-709 and AGT-710 proteins. The latter would be important, for example, as a means for screening for levels of AGT-701, AGT-702, AGT-704, AGT-705, AGT-706, AGT-707, AGT-708, AGT-709 and AGT-710 in a cell extract or other biological fluid or purifying AGT-701, AGT-702, AGT-704, AGT-705, AGT-706, AGT-707, AGT-708, AGT-709 and AGT-710 made by recombinant means from culture supernatant fluid. Techniques for the assays contemplated herein are known in the art and include, for example, sandwich assays and ELISA.

It is within the scope of this invention to include any second antibodies (monoclonal, pqlyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of AGT-119, AGT-120, AGT-121, AGT-122, AGT-422, AGT-123 and AGT-504.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of AGT-119, AGT-120, AGT-121, AGT-122, AGT-422, AGT-123 and AGT-504, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffinan, Basic Facts about Hybridomas, in Compendium of Immunology Vol. II, ed. by Schwartz, 1981; Kohler and Milstein, Nature 256: 495-499, 1975; Kohler and Milstein, European Journal of Immunology 6: 511-519, 1976.)

Another aspect of the present invention contemplates a method for detecting AGT-701, AGT-702, AGT-704, AGT-705, AGT-706, AGT-707, AGT-708, AGT-709 and AGT-710 or a derivative or homolog thereof in a biological sample from a subject, said method comprising contacting said biological sample with an antibody specific for AGT-701, AGT-702, AGT-704, AGT-705, AGT-706, AGT-707, AGT-708, AGT-709 and AGT-710 or their antigenic derivatives or homologs for a time and under conditions sufficient for a complex to form, and then detecting said complex.

The presence of the complex is indicative of the presence of AGT-701, AGT-702, AGT-704, AGT-705, AGT-706, AGT-707, AGT-708, AGT-709 and AGT-710. This assay may be quantitated or semi-quantitated to determine a propensity to develop obesity or other conditions or to monitor a therapeutic regimen.

The presence of AGT-701, AGT-702, AGT-704, AGT-705, AGT-706, AGT-707, AGT-708, AGT-709 and AGT-710 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-AGT-701, AGT-702, AGT-704, AGT-705, AGT-706, AGT-707, AGT-708, AGT-709 and AGT-710 complex, a second antibody specific to the AGT-701, AGT-702, AGT-704, AGT-705, AGT-706, AGT-707, AGT-708, AGT-709 and AGT-710, labeled with a reporter molecule capable of producing a detectable signal, is then added and incubated, allowing time sufficient for the formation of another complex of antibody-AGT-701, AGT-702, AGT-704, AGT-705, AGT-706, AGT-707, AGT-708, AGT-709 and AGT-710-labeled antibody. Any unreacted material is washed away, and the presence of AGT-701, AGT-702, AGT-704, AGT-705, AGT-706, AGT-707, AGT-708, AGT-709 and AGT-710 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain AGT-701, AGT-702, AGT-704, AGT-705, AGT-706, AGT-707, AGT-708, AGT-709 and AGT-710 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing, the polymer-antibody complex to the solid surface which is then washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of AGT-701, AGT-702, AGT-704, AGT-705, AGT-706, AGT-707, AGT-708, AGT-709 and AGT-710. The second antibody is linked to a reporter molecule which is used to indicate the binding of the second antibody to AGT-701, AGT-702, AGT-704, AGT-705, AGT-706, AGT-707, AGT-708, AGT-709 and AGT-710.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody absorbs the light energy, inducing a state to excitability in the molecule, followed by emission of the light at a characteristic colour visually detectable with a light microscope. As in the EIA, the fluorescent-labelled antibody is allowed to bind to the first antibody-hapten complex. After washing off the unbound reagent, the remaining tertiary complex is then exposed to the light of the appropriate wavelength. The fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed.

The present invention also contemplates genetic assays such as involving, for example, PCR analysis to detect AGT-701, AGT-702, AGT-704, AGT-705, AGT-706, AGT-707, AGT-708, AGT-709 and AGT-710 or their derivatives.

Real-time PCR is also particularly useful for assaying for particular genetic molecules.

The present invention is further described by the following non-limiting Examples.

### EXAMPLE 1

### Psammomys obesus

In the following examples, Psammomys obesus rats were used for differential expression studies under different conditions. The rats are divided into three groups, based on metabolic phenotype, as follows:
Group A animals: lean
Group B animals: obese, non-diabetic
Group C animals: obese, diabetic.

### EXAMPLE 2

### Sequence of Psanimoinys obesus AGT-701

AGT-701 was identified using microarray analysis of red gastrocnemius muscle in exercise trained and control P. obesus.

The nucleotide sequence is as follows:

[SEQ ID NO:1]

GGACATCTTTTCAGCCATGAGGAGCTTTCTGGAAACTCGGAGTTGATACA
GAAATATAGGAATATAATCACGCAGGCTCCTAACCTGGAGAACATTGAGC
TGTACTGGAACAGCTACAACAACCGCCGAGACCTGAACTTCGAGCGAGGT
GGTGAGATGACCCTCAAGTGCCCTGTGATGCTGGTGGTAGGAGACCAAGC
GCCTCATGAGGATGCCGTGGTGGAGTGTAACTCAAAACTGGACCCCACAC
AGACCTCGTTCCTCAAGATGGCTGATTCTGGAGGTCAGCCACAGCTGACC
CAGCCAGGCAAGCTGACTGAGGCTTTCAAGTACTTNCTGCAAGGCATGGG
CTACATGGCCTCCTCCTGCATGACTCGCCTATCGAGGTCTCGCACGGCAT
CTTTGACCAGCGCAGCATCCATTGAT

### EXAMPLE 3

### AGT-701 Sequence Homology

AGT-701 demonstrated sequence homology to N-myc downstream-regulated gene 2 (NDRG2)

### EXAMPLE 4

### AGT-701 Gene Expression

NDRG2 is a cytosolic protein of 371 amino acids with a molecular mass of 40.7 kDa (Zhou et al., Genomics 73(1): 86-97, 2001) and is encoded by a 2.4 kb mRNA. By radiation hybrid analysis, Kalaydjieva et al. (Am. J. Hum. Genet. 67(1): 47-58, 2000) mapped the NDRG2 gene to chromosome 14q11.2. No information currently exists on the function of NDRG2. The human and mouse NDRG2 proteins are 92% identical (Zhou et al., 2001). Rat NDRG2 was recently identified and has approximately 90% homology to the mouse and human protein (Boulkroun et al., J. Biol. Chem. 277(35): 31506-31515, 2002).

NDRG2 is part of the NDRG family, which includes NDRG1, NDRG3 and NDRG4. At the amino acid level, the four members share 53-65% identity (Kalaydjieva et al., 2000; Zhou et al., 2001). NDRG1 and NDRG3 belong to one subfamily and NDRG2 and NDRG4 to another. The N— and C-terminal regions are the most divergent regions between the four NDRG proteins, however the C-terminal five aa residues, Met-Glu-Val-Ser-Cys-COOH [SEQ ID NO:12] are conserved in all human and mouse proteins. There are three tandem repeats of GTRSRSHTSE [SEQ ID NO:13] in the C-terminal region of NDRG1 which are not present in NDRG2, NDRG3 and NDRG4.

All members of the NDRG family are cytosolic proteins (Qu et al., Mol. Cell. Biochem. 229(1-2): 35-44, 2002). Each of the four proteins contains an α/β hydrolase fold which is common to a number of hydrolytic enzymes, suggesting that NDRG2 may have an enzymatic function (Boulkroun et al., 2002). Hydrolases are enzymes that catalyze the hydrolysis of various bonds, e.g. C—O, C—N, C—C, phosphoric anhydride bonds, etc. Hydrolysis is the rupture of one or more chemical bonds by reaction with, and involving the addition of, the elements of water.

NDRG1 was the first of the family identified and has been shown to be involved in stress responses, hormone responses, cell growth, and differentiation (Zhou et al., 2001 and references therein). NDRG1 gene expression is up-regulated by many agents, such as reducing agents, tunicamycin, lysophosphatidylcholine, okadaic acid, calcium ionophore, DNA damaging agents, nickel compounds, forskolin, and androgens. The gene is also up-regulated during cell differentiation, in response to hypoxia, and at certain stages of the cell cycle in a p53-dependent manner. Over-expression of NDRG1 in tumor cells decreases the proliferation rate, enhances differentiation, and suppresses the metastatic potency of the cancer cells. In contrast, NDRG1 is repressed by N-myc and c-myc and in many tumor cells. A nonsense mutation in the NDRG1 gene is causative for hereditary motor and sensory neuropathy-Lom (HMSNL), a severe peripheral neuropathy characterized by Schwann cell dysfunction and progressive axonal loss in the peripheral nervous system. This suggests that NDRG1 functions in the peripheral nervous system necessary for axonal survival.

After the identification of NDRG1 in human and mouse, two other members of the family were then identified in mouse, Ndr2 and Ndr3 (Okuda and Kondoh, Biochem. Biophys. Res. Commun. 266(1): 208-215, 1999). In contrast to NDRG1, Ndr2 and Ndr3 were not under negative regulation by N-myc. Their expression during mouse development indicates that the three members of the family are under distinct spatio-temporal regulations, implying that genes of the NDRG family probably have tissue-dependent allotments of the possibly related functions (Okuda and Kondoh, 1999). Using the novel mouse sequences of Ndr2 and Ndr3 to search the human genome databases, Kalaydjieva et al., 2000 identified the homologous human genes, which they referred as NDRG2 and NDRG3. Zhou et al., 2001 cloned NDRG3 and NDRG4, studied the human NDRG gene family and further characterized NDRG4.

NDRG1, NDRG2 and NDRG3 are all expressed in a wide variety of tissues. NDRG2 is most highly expressed in adult skeletal muscle, brain and heart (Zhou et al., 2001; Qu et al., 2002), and NDRG3 is most highly expressed in brain and testis (Zhou et al., 2001; Qu et al., 2002). NDRG4 is specifically expressed in brain and heart (Zhou et al., 2001; Qu et al., 2002).

Zhou et al., 2001 identified two forms of NDRG2, with and without a 14 amino acid insertion in the N-terminal region, located after arnino acid number 25. This insertion was also present in mouse Ndr2. They designated the form with the insertion NDRG2 and the form without the insertion NDRG2^{var}. Rat NDRG2 has four isoforms (Boulkroun et al., 2002). They differ in their 5′UTR sequence, which are either 87 or 50 nucleotides in length, and the presence or absence of the 42 base pairs (14 amino acids) insertion in the coding sequence at the same site as in human and mouse. The proteins are 357 or 371 amino acids in length. The insertion corresponds to the inclusion of exon 3 of the human DRG2 gene, suggesting an alternative splicing event. Sequences highly homologous to the 2 rat NDRG2 5′UTR were found on different exons on the human NDRG2 gene, indicating that they might correspond to alternative 5′ untranslated exons (Boulkroun et al., 2002). This genomic organization strongly suggests the presence of alternative promoters which could direct expression of NDRG2 in a tissue specific and developmentally regulated manner.

NDRG2 has 34% identity to Drosophila MESK2, a component of the Ras pathway (Boulkroun et al., 2002). Ras is an upstream regulator of phosphatidylinositol 3 kinase, and it may be hypothesized that NDRG2 may affect skeletal muscle insulin signalling through that pathway.

A Glucocorticoid Responsive Element (GRE) half-site (TGTTCT) is present in the human NDRG2 promoter (Boulkroun et al., 2002). However, the glucocorticoid dexamethasone did not alter NDRG2 expression in RCCD2 cells, a rat kidney cortical collecting duct cell line, while the glucocorticoid-regulated gene sgk was induced.

In conclusion, NDRG2 belongs to a family of genes putatively involved in growth arrest and induction of cell differentiation, the Ras pathway, and the peripheral nervous system. Although it has strong homology to NDRG1, it is not under negative regulation by N-myc. NDRG2 is a cytosolic protein, probably with an enzymatic function.

### EXAMPLE 5

### Red Gastrocnemius Muscle: Exercise Training

AGT-701 gene expression in skeletal muscle of P. obesus increased with exercise training (p<0.001), and was negatively correlated with blood glucose (R²=0.2872, p=0.015) and the change in blood glucose after training (R²=0.2291, p=0.033). AGT-701 expression also correlated positively with energy expenditure (R²=0.416, p=0.003).

### EXAMPLE 6

### Red Gastrocnemiuis Muscle: Fasting (24 h)

AGT-701 expression was significantly lower in C fed P. obesus, compared to A fed animals (p=0.011) and significantly higher in B fasted P. obesus compared to A fasted (p=0.04) and C fasted animals (p=0.03). AGT-701 expression negatively correlated with body fat (R²=0.1803, p=0.043), body weight (R²=0.2672, p=0.012) and blood glucose (R²=0.1865, p=0.04) in the fed P. obesus.

### EXAMPLE 7

### Sequence of Psammomys obesus AGT-792

AGT-702 was identified using microarray analysis of red gastrocnemius muscle in exercise trained and control P. Obesus.

The nucleotide sequence is as follows:

[SEQ ID NO:2]

GCTGGTACCGGTCCGGAATTCCCGGGATATCGTCGACCCACGCGTCCGGT
GGTGGAGAAGATCGCTCCTGCCGTGGTTCACATTGAACTGTATCGCAAAC
TTCCTTTCTCGAAGAGGGAGGTGCCAGTGGCGAGTGGGTCCGGATTTATC
GTGTCTGAGGATGGACTGATTGTGACCAATGCTCACGTGGTGACCAACAA
AAACAGGGTCAAGGTTGAGCTGAAGAATGGAGCAACCTATGAAGCTAAAA
TCAAGGATGTGGATGAAAAGGCAGACATCGCACTTATCAAAATTGACCAC
CAGGGAAAGCTGCCAGTCTTGCTGCTGGGCCGCTCCTCAGAGCTTCGACC
AGGAGAGTTTGTGGTCGCCATCGGAAGCCCCTTTTCCCTTCAAAACACAG
TCACCACTGGGATCGTCAGTACCACCCAGCGAGGCGGCAAAGAGCTGGGG
C

### EXAMPLE 8

### AGT-702 Sequence Homology

AGT-702 demonstrated sequence homology to Protease, serine 11 (PRSS11).

### EXAMPLE 9

### Gene Expression as Measured by SYBR Green Real Time PCR: Red Gastrocnemius Muscle; Exercise Training

AGT-702 gene expression in skeletal muscle of P. obesus increased after exercise training (p<0.001) and was negatively correlated with body weight (R²=0.4538, p=0.008) in exercise trained P. obesus. AGT-702 expression was positively correlated with energy expenditure (R²=0.2823, p=0.019), and negatively correlated with blood glucose (R²=0.3903, p=0.006). and the training induced change in blood glucose (R²=0.1987, p=0.007) in all P. obesus.

### EXAMPLE 10

### Sequence of AGT-704 Psammomys obesus

AGT-704 was identified using microarray analysis of red gastrocnemius muscle in exercise trained and control P. obesus.

The nucleotide sequence is as follows:

[SEQ ID NO:3]

TGACATTTTCTTTCCACCTCTTATGATAGCTGATATATACTAAATCTTTA
TACAGAAATGTCAGTACTTGAACAAATTCAAAACACATTGGTTTATTAAC
TTTTGGCTCATGCATGGTTTATTAGGTTCAAATTATACCTGATTCATCTA
TATTTACTTTTAAAATGTGTGGTTTCCTCATTTTAAAAGTAAAACTAAAC
AGTGCTTTTGGAATTTCTAAGCTACTAATTGTTGATAGATACAGCCTGTG
TCTAGTAAAATAGTTTTGTGGGTGTGGGTTCTATCTTTCCATGAAAAAGT
GGGAGGTGTAAGTTAGTTTGGTTAGTGCCTAATAGTTAAATTTATATAAA
ATAAGAATGAGCATTTGGTATCTGTATGAAAGGGCCCTAAATCAAAATGA
TTATCCATAATCAATCTTTATTCTTGTTTTATAAAAACCAAAGGGCACTC
ATTGGTTAAGTGTGCTGAGATAGAAAAG

### EXAMPLE 11

### AGT-704 Sequence Homology

AGT-704 demonstrated sequence homology to Mus musculus RIKEN cDNA 1200009K13 gene (1200009K13Rik), mRNA. There are no human matches with the P. obesus sequence. However when BLASTing the mouse sequence NM_{—}025814 against the NR database, it matches strongly to Homo sapiens CGI-55 protein MRNA and Homo sapiens PAI-1 mRNA-binding protein (PAI-RBP1). These are the same gene with the LocusLink and Unigene cluster calling it PAI-RBP 1.

### EXAMPLE 12

### AGT-704 Gene Expression

PAI-RBP1 is a 387 amino acid protein (with additional six and/or 15 amino acids insert in some variants) that plays a role in regulation of mRNA stability. Regulation of mRNA stability is an important component of the regulation of gene expression and is known to have a significant role in normal physiology and development.

The PAI-RBP1 protein binds to an A-rich region in the 3′ 134 nucleotides of the PAI-1 mRNA. This 134 nucleotides region is able to confer cyclic nucleotide regulation of mRNA stability and is, therefore, called the CRS (cyclic nucleotide-responsive sequence) The PAI-1 CRS includes a 75 nucleotide U-rich region at its 5′ end and a 24 nucleotides A-rich region at its 3′ end. Mutation of the A-rich portion reduces binding by PAI-RBP1 and eliminates cyclic nucleotide regulation of mRNA decay.

The amino acid sequence of PAI-RBP1 includes an RGG box at amino acid 343-359, as well as an Arg-rich (amino acid 126-137) and an RG-rich (amino acid 163-184) motif, which places it in the general category with RNA-binding proteins, even though it does not have other RNA binding motifs such as an RNA recognition motif (RRM) or K-homology (KH) domain. The potential protein kinase A phosphorylation site (RKES) at serine 74 is also important given that this protein could be regulated by cyclic nucleotides.

PAI-RBP1 includes blocks of sequence that are highly conserved in a number of metazoans including mammals, birds, Drosophila and Arabidopsis. Thus, PAI-RBP1 identifies a family of proteins with a previously unidentified domain that may define a new RNA-binding motif.

PAI-RBP1 has four splice variants, from two alternative splice sites, in both human and rat. An insertion of six amino acids after position 202 is found in some transcripts, and an insertion of 15 amino acids after amino acid 226 is found in some, both with or without the six amino acid insert.

PAI-RBP1 mRNA is expressed in a wide variety of tissues suggesting that it has a more general biological role involving regulation of mRNA stability or processes requiring interaction with RNA.

Plasmin is a broad spectrum protease. It is the major fibrinolytic enzyme in blood and also participates in a number of physiological and pathological processes involving localized proteolysis. Plasminogen is converted to plasmin by plasminogen activators (PAs), which are serine proteases and hydrolyze one peptide bond of plasminogen. Plasminogen activator activity is regulated by plasminogen activator inhibitor 1 (PAI-1). It is the mRNA of PAI-1 that PAI-1-RBP1 binds to. PAI-1 expression is also regulated by growth factors, cytokines and hormones including agents that regulate cAMP levels.

PAI-1 is consistently elevated in obesity and type 2 diabetes (ertens and Van Gaal, Obes. Rev. 3(2): 85-101, 2002). There is a strong positive correlation between this elevated PAI-1 and the degree of hyperinsulinemia. Both modest and substantial weight loss have been found to significantly reduce PAI-1 levels. Recently it has been demonstrated that the adipocyte itself is able to produce PAI-1. Only the abdominal fat, not femoral subcutaneous fat, PAI-1 gene expression contributes to increases in plasma PAI-1 in obesity (Mavri et al., Diabetologia 44(11): 2025-2031, 2001). Adipose tissue also produces several effector molecules that can up regulate PAI-1. These molecules include transforming growth factor β, TNF α, angiotensin II and interleukin 6. Insulin stimulates PAI-1 gene expression but glucose transport and PAI-1 gene expression are mediated by different insulin signaling pathways (Samad et al., Mol. Med. 6(8): 680-692, 2000). The disturbances in the haemostatic and fibrinolytic systems in part explains why obese and type 2 diabetic patients are at risk for the development of cardiovascular diseases. Increased PAI-1 levels in the blood vessel wall decreases local fibrinolysis which may elevate thrombus formation and the evolution of atherosclerotic plaques (Pandolfi et al., Arterioscler Thomb. Vasc. Biol. 21(8): 1378-1382, 2001). Chronic inflammation has emerged as a new risk factor for the development of type 2 diabetes. Elevated levels of acute-phase proteins and PAI-1 predict type 2 diabetes independent of insulin resistance and other known risk factors for diabetes (Festa et al., 51(4): 1131-1137, 2002).

A 4G/5G polymorphism in the PAI-1 promoter is strongly linked to obesity, and a markedly increased risk for obesity is associated with the 4G allele in its homozygous form (Hoffstedt et al., Diabetologia 45(4): 584-587, 2002). Regular exercise has been shown to be effective for controlling elevated PAI-1 levels in subjects homozygous for the 4G allele (Vaisanen et al., Thomb. Haemost. 82(3): 1117-1120, 1999).

### EXAMPLE 13

### Gene Expression as Measured by SYBR Green Real Time PCR: Red Gastrecnemitis Muscle; Exercise Training

AGT-704 gene expression in skeletal muscle of P. Obesus increased with exercise training (p<0.001). AGT-704 gene expression negatively correlated with blood glucose (R²=0.3903, p=0.006) and positively correlated with energy expenditure (R²=0.4767, p=0.001) in P. obesus.

### EXAMPLE 14

### Red Gastrocnemius Muscle; Fasting (24 hr)

There were no differences in AGT-704 gene expression between the fed or the fasted groups of P. obesus, although there was a trend towards decreased expression in C fed P. obesus compared to A fed (p=0.08). However, AGT-704 gene expression showed a significant negative correlation with blood glucose in fed P. obesus (R²=0.3059, p=0.007)

### EXAMPLE 15

### Sequence of AGT-705 Psammomys obesus

AGT-705 was identified using microarray analysis of red gastrocnemius muscle in exercise trained and control P. obesus.

The nucleotide sequence is as follows:

[SEQ ID NO:4]

CGCAGATACTGCAGAGCTGACGCATTCTTTGCCTGGCATCTCAGCTTGCT
AGGTGTCTCATCTTCGCTCTGCGCTCGTGGCCTCCTCCAAGGCCTCCAGT
CTCCTTTAAGAAAACTCAAGACCTGGGAAGCTACGATGCGAGCTTGATGC
CGCTACCCTAGCAGGCTATGGACTTCCTGAGGGTCCTCGGACTGTTGACA
CCCATTCCGATCCGCATCCTTCCAAGCTGATAAGCCCGGGACCCTAGGGC
GGGGTGCCCAGACTCATGTGTGACGCCTTGCAGTGAAACCCCATTCCCAG
TGTGGTTGCTTCTTTGCTGGGCTTTGGCCCATTTGATACCACGAAGGATG
ACGATGCTAGTTATGCAGCAGCCAACACACCTCCCCCCAACTCTCCGCTG
TCACTGGTGGGCCCCACTGTCCAGGAAGCAGGTGTCCGGAACTGACATCT
TGGAGCAGAGGGGCCATGAGAGGTGTGTGTATCCTGCCAGAAAGCAGCTG
GACCACGACGCTCCCAAGATGAACCCACTGTATACAGAGGCATCATGGGA
GTTGTTATGTCAGGAGCATTCTAGACCCACGTGTACTTGAGCGTGGAAAG
ACAGAAGANANGCGCAGAGACTGGGGCACTTGATCTGCTCACCATGATCG
CCTGCACGGGTCTCATCCAGTTCCTGCCTTAGGCTACAGTGGCGGTGTCC
ACGGGCTTGCCATTCAACGTGCTCTCAGACCCAGATCGGGCTCACCACTG
AGGAGAACCTTTTCACTTTGGTGGGTATGCAGAGGGAAGGGTCTCGACTC
CAGAGACCTGGAGTCCAAAGTTTGTTTTTTTTTATTTACCAGTAATTTAT
TTATTTTTTTTATTATTAGTTATGAGTCCAAAGTTTAATACCATTCAAGC
TACTGCTGTCTGTCTGCTTAGAGCCACAGCATGCAATGTGGCACCAAGGC
ATCCTTGTCCTCACAGTTTCACACTGTGGGAACAGGCATCCTTGTTCTTA
CAGATTAGCGCGAGGGAAACCAGAAATATTAAACACGCAGGGTTGTCTCT
CCAAAGGGAGAGGCACATACCCTGTTTTCCTCCCGAAGGCTGGGAGCGGA
GGTGTTTGATATCCTGGCTACCTCTGTGCAATCTGTAGGCCATGTCCTTA
AGATGTAGCTGTCAGTCGGTAGTGGAGCCGGAGCCGTCAGTCAGTAGATT
GGGGTTGTGGCATGCGCCTTTAACTCCATTTAATTCCAGCACTCTAGTGG
TTTGGTACAGCAGCAGCAGCAGCGGTTGCAGTGGCCCGGGGAAGTCCTGA
AGACCAGCTTTCATCCCAGCACTCAGGCAGCAAAGGCGGGTGGATTCTCT
GAGTTCGAGGCCGGCCTGGTCTACAGAGTGAGTCCAGNCNAGCCAGGGCT
ACAACAGAGAAANCCTCTCTATTGAAAAATAAATAAATTATAAAAAAAAA
AAGGTGTCATGTGTCCTGTGTACTTTACAAAGAATGTTGATGCTTAAGCT
TTTTTGTGCACNCAAGAAAATTGTTTAACTGGTGTCAGACTCCTGAAGTT
TGAACCAGCACTTAGCCNGGCGTGGTGGCGCACGCCTGTAATCCCAGCCC
TCGGGAGGCAGAGGCAGGTGGATCTCTGAGTTAGAGGCCAGCCTGGTCTA
CAGAGTGAGTCCAGGACAGCCAGGATTACACAGAGAAACCCCGTCTCAAA
AATGTAAAATAAATTAAAATAAAGTTTGAACCAACAGTGTTTACTGAGTC
GTGTTGAAACAGATTACCTTTTTGCTTCTCTTTGATCATTATTCTACTGT
GGTGTCAGCAGAGACCCCTCCAGCAGGTGGCCAACGTGAGAGTCTCAAGC
CCGAGAAGGTAAGAATTTAAAAAAAAAAAAAAAA

### EXAMPLE 16

### AGT-705 Sequence Homology

The full clone sequence of AGT-705 matches a mouse mRNA clone BC030414 (not full length mRNA) but no human sequences on the GenBank database.

### EXAMPLE 17

### Gene Expression as Measured by SYBR Green Real Time PCR: Red Gastrocnemius Muscle; Exercise Training

AGT-705 expression was not different between the exercise trained and the control P. obesus, although there was a tendency for expression to increase with exercise training (p=0.35).

### EXAMPLE 18

### Red Gastrocnemius Muscle; Fasting (24 hr)

In this study, there was no difference in AGT-705 expression between the fed P. obesus, however, there was a trend towards increased expression in the C fed P. obesus. AGT-705 expression was significantly higher in the B fasted group when compared with the A fasted group (p=0.035) and the C fasted group (p=0.007) and gene expression was negatively correlated with blood glucose in the fasted P. obesus (R²=0.3701, p=0.027).

### EXAMPLE 19

### Sequence of AGT-706 Psammomys obesus

AGT-706 was identified using microarray analysis of red gastrocnemius muscle in exercise trained and control P. obesus.

The nucleotide sequence is as follows:

[SEQ ID NO:5]

GTTGGGAAAGAATGAAGAAACAACCCGATGAATAGAAATGAAAAGCCTAA
GCCAAATGGATTTCTGTTGAGATGTTGGATGAAAACAAGTATCCACTGTT
TACCAACTTGACGAAAAATCTCAACTGAGGTTTGGCTGTTAAAAAAAAAA
ATTCACTGTGGCCTCTGTGCTTAATTGTCGTAAACCATTGTGACTGTTAC
TGCTCAAAGTATCGTACTGTTCATTAGTAACTACATCAGAATTGCACCGC
TGCTGTTGGAAAAGCCAATAAAGAAACCCCCAGACTGCTGCTCAGCAAAT
GTTAATAAAGTGTGCGCACCGTAGGCCTGTCCACCCAGTCACCAAGCAGC
GTCCCTTTGTCTGCGAGTGGCTGTGGGTGTGATTNACCACCTCAGAGGTG
CACAGCACCTGCTTGNGCCCTTAAGTGTGNGTCAGAAGACAAGCAGCTTC
TCGGTAACCAACAACCTGCTTTTCGGAGCTCAGTGTTTAGGCTGTTTACT
GAATCANATATGTAACTCAGCACACATAAGCGAAGAGAGATTTTGGCTGC
ACTGGCAAGAGTGAACCAAATTTACTTCTATTTTTTAAAGGCAGATCATA
TTTAAGCATATAAGTAATTTATGGATATAAATTGTTGGATATTTATTTTA
GTCTGAATATTTGTTTTTAAATTATTACATGTGTTCTCTATGTCTTTATC
TCTGGAATAACGATGCCATTAACCACATGGCCATATGTTTTGAAAGTTGG
GTGNAACAGAGGAAAAGTCATCCTTCTTGGTTCTTGACTCCCTTTCCTCA
ACTACATGATAAGTCTATCAATAAAGCATTTGACCTCAGCAGGGGCAGAA
GCCTGNAAAGTTAGAAAACTCATTGACCACAGTAGACAATTGATTTCTTA
GAAATAAGAAGTGAGAAGCAGCTGCTGNGCTGAGCAGGGGATGTAAACCA
AGTCCAGATGCACCAACGTGAAGAGGCTTNTAGCAAAAATATGTTTGCCT
CTCACCCCTGCACATGTTCTAGATGCTTAAAAACAGCCACATGGCCCCGC
TGCGAGGACCTCGTAATGTTGTTGTTGTTGTTGTTGTTGTTAAAGGAGTT
CTCACAAGCGTACAAGTGCAGCACTGAAAGTGGCTGAGGCCCACAGTCCT
CAGCACCCAAGTCTNTTCCGCAGCACGCCAAGCTGGTGTTGTCCGGGTGN
GTATGTCTGTGCTCAGTGCCAAGCTGGTGTTTGGTCCCGTTGTATATTAT
GTGCCCCAAGTGTTTTGGGGCANAGCTGACCCANGCTGGACACACTTCTT
TTNGNCTTCGAGTTTACTGGTTGATNCAGNTAAAAATAAATTAATTAATT
AAAGACTT

### EXAMPLE 20

### AGT-706 Sequence Homology

AGT-706 demonstrated sequence homology to Human hypothetical protein FLJ20069 and mouse Ahi-1 (also called mouse 1700015F03).

### EXAMPLE 21

### AGT-706 Gene Expression

The Ahi-1 locus was initially identified as a common helper provirus integration site in Abelson pre-B-cell lymphomas and shown to be closely linked to the c-myb proto-oncogene. Proviral integration within the Ahi-1 region has also been shown in thymomas of T cell origin.

Jiang et al. (J. Virol. 76(16): 9046-9059, 2002) identified the murine gene (Ahi-1) targeted by these provirus insertional mutations. The Ahi-1 cDNA encodes a 1,047-amino-acid protein. The predicted Ahi-1 protein is a modular protein that exhibits several features of a signaling molecule. It contains one SH3 motif and seven WD40 repeats. The Ahi-1 gene is conserved in mammals and encodes two major RNA species of 5 and 4.2 kb and several other shorter splicing variants. The Ahi-1 gene is expressed in mouse embryos and in several organs of the mouse and rat, notably at high levels in the brain and testes.

The Ahi-1 proviral insertions were found at the 3′ end of the gene, in an inverse transcriptional orientation, with most of them located around and downstream of the last exon, whereas another insertion was within intron 22. In addition, another previously identified provirus insertion site, Mis-2, was found to map within the 16th intron of the Ahi-1 gene. In tumor cells harboring insertional mutations in Ahi-1, truncated Ahi-1/viral fused transcripts were identified, including some splicing variants with deletion of the SH3 domain.

In summary, Ahi-1 encodes a protein that exhibits several features of a signaling molecule and is targeted by provirus insertion. Ahi-1 may play an important role in signal transduction in normal cells and may be involved in tumor development, possibly in cooperation with other oncogenes (such as v-abl and c-myc) or with a tumor suppressor gene Nf1).

### EXAMPLE 22

### Gene Expression as Measured by SYBR Green Real Time PCR: Red Gastrocnemius Muscle; Exercise Training

AGT-706 gene expression increased in skeletal muscle of P. obesus after exercise training (p=0.001). In addition, AGT-706 gene expression was negatively correlated with blood glucose (R²=0.3903, p=0.012) and positively correlated with insulin (R²=0.2213, p=0.036) and energy expenditure (R²=0.4031, p=0.003) in P. obesus.

### EXAMPLE 23

### Red Gastrocnemius Muscle; Fasting (24 hr)

In this study, AGT-706 gene expression was significantly higher in the B fasted and C fasted groups, when compared to the A fasted group (p<0.001 for both comparisons). AGT-706 gene expression was negatively correlated with blood glucose in fed P. obesus, (R²=0.2228, p=0.003) and positively correlated with insulin in fasted P. obesus (R²=0.2469, p=0.026).

AGT-706 gene expression tissue distribution was examined in Psammomys obesus. Relative to the hypothalamus, expression was highest in the muscles, testes and ovary, however, detectable levels of gene expression were seen in most tissues.

Expression of AGT-706 in liver cDNA decreased after fasting for 24 h (p=0.001, t test). A positive correlation between AGT-706 gene expression in the liver and plasma insulin concentration was observed (R²=0.1236, p=0.026).

In mesenteric fat, AGT-706 gene expression was significantly elevated in obese animals (p=0.004).

Positive correlations between AGT-706 gene expression in mesenteric fat and body weight (R²=0.13, p=0.01) and insulin (R²=0.1761, p=0.004) were observed.

AGT-706 gene expression was examined in L6 muscle cells treated with increasing concentrations of glucose for 24 h. When cells were incubated in 17.5, 25 or 35 mM glucose, AGT-706 gene expression was significantly elevated compared with cells incubated in 5 mM glucose (p<0.04).

### EXAMPLE 24

### Sequence of AGT-707 Psammomys obesus

AGT-707 was identified using microarray analysis of red gastrocnemius muscle in exercise trained and control P. obesus.

The nucleotide sequence is as follows:

[SEQ ID NO:6]

GTNGAAGCNTAGGAGTTCGAGGATGCGCCCGATGTCGAGCCGCTGGAACC
CACGCTTAGCAATATCATCGAGCAGCGCAGCCTTAAGTGGATCTTCGTCG
GGGGCAAGGGTGGCGTTGGTAAGACCACCTGCAGCTGCAGCCTGGCGGTC
CAGCTGTCTAAGGGACGTGAGAGTGTTCTAATCATTTCCACAGACCCAGC
TCACAACATCTCAGATGCATTTGACCAGAAGTTCTCCAAGGTGCCTACCA
AGGTCAAAGGCTATGACAACCTCTTTGCTATGGAGATAGACCCGAGCCTG
GGCGTGGCAGAGCTCCCTGATGAAGTTCTTCGAGGAAGACAACATGCTGA
GCATGGGCAAGAAGATGATGCAGGAGGCCATGAGCGCCTT

### EXAMPLE 25

### AGT-707 Sequence Homology

AGT-707 demonstrated sequence ASNA1: Human homolog of bacterial arsA arsenite transporter, ATP-binding.

### EXAMPLE 26

### AGT-707Gene Expression

ASNA1 is the human homolog of the bacterial arsA gene. In E. coli, ArsA ATPase is the catalytic component of a multi-subunit oxyanion pump that is responsible for resistance to arsenicals and antimonials. The E. coli ars operon contains two regulatory (arsR and arsD) and three structural genes (arsA, B and C). The arsA gene codes for an oxyanion ATPase that associates with the protein encoded for by arsB, the channel-forming transmembrane protein. Together, the two proteins transport arsenite and antimonite out of the cells across the plasma membrane.

Human ASNA1 encodes a 332-amino acid polypeptide having an N-terminal ATP-binding cassette (ABC) domain and a C-terminal domain of unknown function (Kurdi-Haidar et al., Genomics 36: 486-491, 1996). The protein sequence is highly homologous throughout both domains to hypothetical arsA proteins of C. elegans and yeast. Southern blot analysis indicated the existence of two closely related ARSA genes in the human genome. The existence of a second human ARSA protein was further supported by Western blot analysis, which demonstrated that anti-ARSA1 antibodies identify two proteins of 37 and 42 kD. Kurdi-Haider et al., 1996 expressed ASNA1 and found that the resulting 37-kD protein had ATPase activity.

Northern blot analysis revealed that the ASNA1 gene is expressed in a variety of tissues, with highest expression in the cardiac and skeletal muscle (Kurdi-Haider et al., 1996). Immunohistochemical analysis of normal human tissues detected ASNA1 only in the epithelial cells of the liver, kidney, and stomach wall, in the adrenal medulla, in the islet cells of the pancreas, in the red pulp of the spleen, and in cardiac and skeletal muscle (Kurdi-Haidar et al., J. Histochem. Cytochem. 46: 1243-1248, 1998d). In skeletal muscle the fibers were strongly positive. Interestingly, ASNA1 levels were markedly increased in breast fibroadenomas and carcinomas.

ASNA1 shows a cytoplasmic, perinuclear, and nucleolar distribution (Kurdi-Haidar et al., J. Biol. Chem. 273: 22173-22176, 1998a; Kurdi-Haidar et al., J. Cell. Biochem. 71: 1-10, 1998b). Since the nuclear membrane and the nucleolus were enriched for ASNA1, with no detectable protein in the nucleoplasm, suggests that the nuclear ASNA1 is bound and does not diffuse freely. The cytoplasmic ASNA1 is soluble. The ASNA1 at the nuclear membrane was associated with invaginations into the nucleus in interphase cells. These results and the fact that it is not found in the plasma membrane suggest that ASNA1 is a paralog rather than an ortholog of ArsA and that it probably plays a different role in human cells than does the ArsA protein in bacteria. In human cells it appears to play a role in the nucleocytoplasmic transport of a nucleolar component.

Kurdi-Haider et al., 1998a characterized purified recombinant ASNA1. They determined that the ATPase activity increases in the presence of sodium arsenite (but not antimonite) and that Vmax rather than ATP affinity is enhanced. Human ASNA1 is an arsenite-stimulated rather than an arsenite-dependent ATPase, and has significant basal ATPase activity even in the absence of oxyanions. Kurdi-Haider et al., 1998a found that the active species is likely a dimer or tetramer.

Kurdi-Haidar et al., Somat. Cell Molec. Genet. 24: 307-311, 1998c mapped the ASNA1 gene to chromosome 19q13.3 and determined that it contains four exons and spans 6 kb.

### EXAMPLE 27

### Mouse ASNA1 Gene

Mouse Asna1 encodes a 348-amino acid protein sharing 27% and 99% identity with the E. coli and human proteins, respectively (Bhattachaoee et al., Gene 272: 291-299, 2001). Northern blot analysis detected a 1.3-kb transcript in mouse at highest levels in kidney and testis, moderate levels in brain, liver, lung, and skin, low levels in heart, small intestine, spleen, stomach, and thymus, and negligible levels in skeletal muscle. Bhattachaijee et al., 2001 mapped the mouse Asna1 gene to the C3-D1 region of chromosome 8, and determined that it consists of seven exons spanning over 7 kb.

### EXAMPLE 28

### Gene Expression as Measured by SYBR Green Real Time PCR: Red Gastrocnemius Muscle; Exercise Training

AGT 707 showed increased expression in the red gastrocnemius muscle of exercise trained P. obesus compared to control group animals (p=0.037). There were no correlations between gene expression and other phenotypic variables.

### EXAMPLE 29

### Red Gastrocnemius Muscle; Fasting (24 hr)

There were no differences in AGT-707 gene expression between the fed groups or the fasted groups, although there was a trend for increased expression in group A compared to group B and C P. obesus in the fed state (p=0.075 and p=0.055, respectively). AGT-707 gene expression was negatively correlated with body weight in the fed P. obesus (R²=0.2267, p=0.026).

### EXAMPLE 30

### Sequence of AGT-708 Psanmmomys obesus

AGT-708 was identified using microarray analysis of red gastrocnemius muscle in exercise trained and control P. obesus.

The nucleotide sequence is as follows:

[SEQ ID NO:7]

AAAATTTTACAAATGAGTGTGAATTGCATTCTGATATAATAATTATCACC
CCACCACACTTTTACTGACACTGTTGATGGCCTATGCTGTGTTTTCACAT
CACAATTCTTGTATGGAAAAATTTCTGTGGCCTGTGTAACCCCTCTGGTC
AGTATTATGAAACCAACTATCTTTGGTGATAAATAAGGTTCCGGTAAGAT
GCCCAGGGTTCATGAGTATGGCACAAATAACAGAGGACAGGAGGCCTTCA
CGACGAAGGAGCCCGTAAGTGGCCTGGAGGGCACAGATGCAGTTCCAGGT
CAAGAAAAGAGCAGCTTTTTCAACAGGCAGTCTGTGGGTATGATGGGAAC
TCAGCCTGTCTCTGTAGTTATGGACAGCGTGGCAGGTGACTGTGCCCACA
TCTTCCTATACAGTGCTTTTTTTTTACTGACTGGAAGTACGTGAATCTCA
CTTAGTCCCCAACTGGACGTTTTCTGGAAAAACAAAGCAAATGTTAAAGT
ATGTCTTTCTGGATATAGGCCAGNAGNAAATACATTAAGAATGAGAGGCC
TTGCTTTGATCTCAGCCATTGGAGGCTAGAAAAAAATTGAAAGGAACCTT
CCTGTTGATAGACTCAAAGCCGTGAACAGAAGCCTCTTGGCCTGTTTCAG
ACAATCTCTGGTAATCTACTGACAATATCCAACAGTTTCGATGTCCTTGT
TTAACTACCCTGGTAGCTTTCTTGTGGATTTGAAGTTCATTTTTAAAGCT
GTGGAATTTCAAACTGAATTCACGTGCATTTTGTAAAAGTTCAGAACCAG
TGCTGAGTCTGTGTGGCAGGTTTTTTTCACCGCGTGATATACTATTACAA
ATGCATGTGGTGCCATGCTTGTCTTCAAATATATAAGTAGTGCTAAATGG
ATAAGTCATATGGAGCTTTTGATTTAG

### EXAMPLE 31

### AGT-708 Sequence Homology

AGT-708 demonstrated sequence homology to Protein kinase inhibitor α (PKIα).

### EXAMPLE 32

### AGT-708 Gene Expression

cAMP-dependent protein kinases co-ordinate cellular responses to hormones and neurotransmitters by altering processes such as cell division, membrane permeability and transcription. Most of the effects of cAMP in the eukaryotic cell are mediated through the phosphorylation of target proteins on serine or threonine residues by the cAMP-dependent protein kinase (EC 2.7.1.37). The inactive cAMP-dependent protein kinase is a tetramer composed of two regulatory and two catalytic subunits. The cooperative binding of four molecules of cAMP dissociates the enzyme in a regulatory subunit dimer and two free active catalytic subunits. In the human, four different regulatory subunits (PRKAR1A, PRKAR1B, PRKAR2A, and PRKAR2B) and three catalytic subunits (PRKACA, PRKACB and PRKACG) have been identified.

Members of the cAMP-dependent protein kinase inhibitor family are specific and extremely potent competitive inhibitors of cAMP-dependent protein kinase activity. These proteins interact with the catalytic subunit of the enzyme after the cAMP-induced dissociation of its regulatory chains. The inhibitory site contains regions very similar to the hinge regions (sites that directly interact with the enzyme active site) and “pseudosubstrate site” of the regulatory chains; but unlike these chains, PKI does not contain cAMP-binding sites. The arginine residues within the inhibitory site are essential for inhibition and recognition of the enzyme active site.

### EXAMPLE 33

### Cloning

Using the mouse protein kinase inhibitor-alpha to screen a human neuroblastoma cell line cDNA library, Olsen and Uhler (Molec. Endocr. 5: 1246-1256, 1991) isolated clones encoding human PKIA. The deduced 75-amino acid PKIA protein shares 100% and 97% sequence identity with the rabbit and mouse homologs, respectively. Northern blot analysis detected major 4-kb and minor 2-kb PKIA transcripts in skeletal muscle. Using kinase assays with transfected COS cells, Olsen and Uhler, 1991 verified that the PKIA cDNA produces a heat-stable inhibitor of protein kinase. Protein extracts inhibited both the α (601639) and β (176892) isoforms of the protein kinase catalytic subunit with equal efficacy. Using a transcriptional activation system, Olsen and Uhler, 1991 demonstrated that elimination of a conserved alternative translation start site in PKI increased the inhibitory activity of the PKI expression vector.

### EXAMPLE 34

### Gene Expression as Measured by SYBR Green Real Time PCR: Red Gastrocnemius Muscle; Exercise Training

AGT-708 gene expression in skeletal muscle of P. obesus was negatively correlated with activity (R²=0.3543, p=0.006) and the change in carbohydrate oxidation after exercise training (R²=0.368, p=0.01), and was positively correlated with energy expenditure (R²=0.5377, p<0.001) and the change in fat oxidation after exercise training (R²=0.3987, p=0.007) when all animals were analyzed together. AGT-708 gene expression positively correlated with food intake (R²=0.2996, p=0.043) and the change in fat oxidation (R²=0.3452, p=0.047) and negatively correlated with the change in carbohydrate oxidation (R²=0.395, p=0.021) in the exercise trained P. obesus.

### EXAMPLE 35

### Red Gaswocnemius Muscle; Fasting (24 hr)

AGT-708 gene expression was significantly higher in the B fasted and C fasted groups when compared to the A fasted group (p<0.013 for both groups). AGT-708 gene expression positively correlated with blood glucose in fasted P. obesus (R²=0.2512, p=0.021).

### EXAMPLE 36

### Sequence of AGT-709 Psammomys obesus

AGT-709 was identified using microarray analysis of red gastrocnemius muscle in exercise trained and control P. obesus.

The nucleotide sequence is as follows:

[SEQ ID NO:8]

TGAGATAGCTACTCCATAAGCCTCTGAAGAGCAATAGCTAATTTATTATT
ACTGTAATTNTTTTAAAGGCTTTAAAGTGCCTCGGGGGTTCCTTGAAACT
AATTTTCTACTTCTGGGATTCCCTGGATTCTTTATAAGAGATGGTGACAT
GACTAGGGAAATTCTTTTTTTAGTATGAAAATTGTCCCTTCAATACTTTT
CTCTTACTGGCATTGAATTATCACAGAGACAGAAAATTGGTAATTTTTTT
AATTTCTAACTCTCCCAGAAAACTCCTCTTGCCTAGTATTTATTTGATGT
GCTTTAACCATGGGAGGAGGGGTGGGGGGGGAACTCATTCAAGCTGCCAG
TATTTTGATCTACAACCTGTAGCA

### EXAMPLE 37

### AGT-799 Sequence Homology

AGT-709 demonstrated sequence homology to Human KIAA0663

### EXAMPLE 38

### Gene Expression as Measured by SYBR Green Real Time PCR: Red Gastrocnemius Muscle; Exercise Training

There was no difference in AGT-709 gene expression in exercise trained animals when compared to controls. A significant positive correlation between AGT-709 gene expression and body weight (R²=0.37, p=0.004) and energy expenditure (R²=0.5377, p=0.008) was found when all animals were analyzed together. AGT-709 expression in exercise trained animals showed a positive association with body weight (R²=0.4538, p=0.045), and the change in glucose (R²=0.3198, p=0.035) and in body weight (R²=0.3244, p=0.033) after exercise training

### EXAMPLE 39

### Red Gastrocnemius Muscle; Fasting (24 hr)

AGT-709 expression was significantly lower in the C fed P. obesus than the A fed P. obesus(p=0.049), although there were no differences in gene expression between the fasted groups. AGT-709 expression was negatively correlated with body weight (R²=0.2158, p=0.039) and blood glucose (R²=0.2495, p=0.026) in fed P. obesus.

### EXAMPLE 40

### Sequence of AGT-710 Psammomys obesus

AGT-710 was identified using microarray analysis of red gastrocnemius muscle in exercise trained and control P. obesus.

The nucleotide sequence is as follows:

[SEQ ID NO:9]

CCCACGCAGTCCGGGTGGCTCTGCAGCACAATTTAGGCCTTGGAGGAGCT
GTGGTTGTCACCCTCTACAANATGGGCTTCCCCGAAGCGGNCAGCTCCTT
CAGAACACACCANATTTCGGCTGCTCCCACCAGCTCTGCAAGGGATGGAT
TCAAGGCCAATCTTGTCTTTAAGGAGATCGAGAAGAAGCTTGAAGAGGAA
GGGGAACAGTTCGTGAAGAAGATCGGTGGGATTTTTGCCTTCAAAGTGAA
GGACGGCCCTGGAGGCAAAGAAGCCACCTGGGTGGTGGATGTGAAGAATG
GCAAGGGATCCGTGCTTCCCAACTCAGATAAGAAGGCTGACTGCACAATC
ACCATGGCCGACTCCGACTTGCTGGCTCTGATGACTGNCAAAATGAACCC
TC

### EXAMPLE 41

### AGT-710 Sequence Homology

AGT-710 demonstrated sequence homology to Sterol carrier protein 2 (SCP2)

### EXAMPLE 42

### AGT-710 Gene Expression

Sterol carrier protein 2 (SCP-2, SCP2) is also known as Nonspecific lipid-transfer protein, mitochondrial precursor (NSL-TP) and Sterol carrier protein X (SCP-X, SCPX) (SCP2 Genecard record, Genecards Website, Weizmann Instsitute of Science; Human SCP-2 (NLTP_HUMAN) record froM SWISS-PROT database, ExPasy Website). The SCP-2 gene is a fusion gene that has two initiation sites. The gene encodes two proteins: SCP-2 and SCP-X. Both proteins share the same C-terminal 13 kDa (123 aa) sequence. The SCP-2 transcript encodes a 15 kDa (143 amino acid) pro-SCP-2 protein which is post-translationally cleaved to form the mature 13 kDa SCP-2 protein. The longer isoform, SCP-X, is translated into a 58 kDa (547 amino acid) protein and is partially cleaved to form two proteins—the 13 kDa SCP-2 and a 45 kDa (404 amino acid) protein (Gallegos et al., Prog. Lipid. Res. 40(6): 498-563, 2001). The latter is a 3-ketoacyl-CoA-thiolase specific for branched chain acyl CoAs (Stolowich et al., Cell Mol. Life Sci. 59(2): 193-212, 2002). In most tissues however, the majority of the 58 kDa protein remains intact (Stolowich et al., 2002).

The 13 kDa SCP-2 binds a number of different ligands such as fatty acids, fatty acyl CoAs, cholesterol and phospholipids. It is thought that the 13 kDa SCP-2 facilitates the intracellular transport of lipids such as cholesterol between membranes. SCP-2 has been shown to also interact with a number of other ligands, and other possible physiological functions are being examined (Gallegos et al., 2001).

Related genes proteins that also contain the C-terminal SCP-2 domain are: DHB4 HUMAN: 17β-hydrgxysteroid dehydrogenase IV (DBH4 record fromPfam database of protein families Website, Sanger Centre; Gallegos et al., 2001) (the C-terminal SCP-2 domain is known to be required for peroxisomal import of this protein) and UNC-24 protein from C. elegans (this protein consists of an N-terminal SPFH (or band 7) domain and a SCP-2-like C-terminal domain. The human homologue of this protein is stomatin-like protein (hSLP). Its function is unknown (Gallegos et al., 2001; Barnes et al., J. Neurochem. 67(1): 46-57, 1996).

### EXAMPLE 43

### Chromosomal Location and Gene Structure

The SCP-2 gene is located at chromosome 1p32 (Vesa et al., Hum. Molec. Genet. 3: 341-346, 1994; SCP2 Genecard record, Genecards Website, Weizmann Institute of Science; Sterol Carrier Protein 2 record, OMIM Website). It consists of 16 exons and 15 introns in humans, mice, rats and chickens (Ohba et al., Genomics 24: 370-274, 1994). The mouse homolqgue is found on chromosome 4 (Welch et al., Genome 7: 624-625, 1996). There are 2 promoter regions that initiate at least 4 mRNA species (Stolowich et al., 2002). Two alternatively polyadenylated mRNA transcripts (mRNAs of 2.8 kB and 2.2 kb) encode the 58 kDa SCP-X protein, and two alternatively polyadenylated transcripts (mRNAs of 1.5 kb and 0.9 kb) encode the 15 kDa pro-SCP-2 protein (Stolowich et al., 2002). Another study (Yamamoto et al., Proc. Natl. Acad. Sci. USA 88(2): 463-467, 1991) identified a further two transcripts in the liver (1.8 kB and 3.2 kb species) where the 1.8 kb isoform was most abundant. Little is known about the transcriptional regulation of the gene (Gallegos et al., 2001].

### EXAMPLE 44

### Structural Features

The secondary and tertiary structures for the 13 kDa molecule are available (Stolowich et al., 2002; Szyperski et al., FEBS Lett. 335(1): 18-26, 1993), however no structures are available for the 15 kDa, 45 kDa or 58 kDa molecules. At the protein sequence level the SCP-X protein consists of 3 domains (SCP-2 record from Pfam database of protein families Website, Sanger Center):
Thiolase N-terminal domain (residues 11-240).
Thiolase C-terminal domain (residues 245-402).
SCP-2 at the C-terminal of the protein (residues 433-543).

The mature 13 kDa SCP-2 protein consists of only the SCP-2 domain. A tertiary structure is available for this molecule (Stolowich et al., 2002). Functionally important structural elements that have been identified by elucidation of the structure are:
The N-terminal 32 residues form an amphipathic helix, one face of which is a membrane binding domain that binds to anionic phospholipids at membrane surfaces.
The hydrophobic faces of the N-terminal amphipathic helices plus β-strands 4,5 and β-helix D form a ligand-binding cavity able to accommodate multiple types of lipids (fatty acids, acyl coAs, cholesterol, phospholipids, isoprenoids).
The C-terminus is highly hydrophobic and it is thought to form a hydrophobic cap that closes around the ligand upon binding.

### EXAMPLE 45

### Putative Biochemical Function

Gallegos et al., 2001 and Stolowich et al., 2002 have reviewed the various functions of SCP-2. In summary:
SCP-2 is believed to affect cholesterol synthesis—overexpression in mice of the rat SCP-2 gene by adenoviral infection increased liver cholesterol levels by 70% and decreased liver cholesterol synthesis by 60% (Zanlungo et al., Gastroenterology 119(6): 1708-17-19, 2000).
SCP-2 (Fuichs et al., Biochem. J. 336(1): 33-37, 1998; Publielli et al., Biochem. J. 317(3): 681-687, 1996; Kawata et al., Clin. Chim. Acta 197(3): 201-208, 1991; Fuchs et al., J. Biol. Chem. 276(51): 48058-48065, 2001) and SCP-X (Bun-ya et al., J. Biochem. (Tokyo) 123(2): 347-352, 1998; Ferdinandusse et al., J. Lipid. Res. 41(3): 336-342, 2000; Wanders et al., J. Inherit. Metab. Dis. 21(3): 302-305, 1998) participate in different aspects of bile acid synthesis.
SCP-2 is believed to be involved in transport of cholesterol from ER to bile (Ito et al., Gastroenterology 110(5): 1619-1627, 1996).
SCP-2 and possibly SCP-X and pro-SCP-2 are believed to be involved in triacylglyceride formation (Seedorf et al., Genes Dev. 12(8): 1189-1201, 1998; Atshaves et al., J. Lipid Res. 40(4): 610-622, 1999; Murphy and Schroeder, Biochim. Biophys. Acta. 1345(3): 283-292, 1997; Starodub et al., Am. J. Physiol. Cell Physiol. 279(4): C1259-1269, 2000).
SCP-2 and possible SCP-X and pro-SCP-2 are believed to participate in peroxisomal fatty acid oxidation (Seedorf et al., 1998; Schroeder et al., Biochemistry 34(37): 11919-11927, 1995; Ossendorp et al., Arch. Biochem. Biophys. 334(2): 251-260, 1996).
SCP-2 is thought to facilitate cholesterol transport to mitochondria and be involved in regulating steroidogenesis (Yamamoto et al., Proc. Natl. Acad. Sci. USA 88(2): 463-467, 1991; Yamamoto Hokkaido Igaku Zasshi 67: 839-848, 1992).
The 58 kDa SCP-X is one of three 3-ketoacyl-CoA thiolases found in peroxisomes. SCP-X is believed to play an exclusive role in peroxisomal branched chain fatty acid oxidation (Antonenkov et al., J. Biol. Chem. 272(41): 26023-26031, 1997; Antonenkov et al., Protein Expr. Purif 18(3): 249-256, 2000; Wanderrs et al, Biochem. Biophys. Res. Commun. 236(3): 565-569, 1997) and in oxidation of the branched side chain of cholesterol to form bile acids (Fuchs et al., 2001; Bun-ya et al., 1998; Ferdinandusse et al., 2000).

### EXAMPLE 46

### Scp2 Knockout Mice

scp2 null mice show a severe block at the level of thiolytic cleavage in pristanic acid β-oxidation and lack normal peroxisomal degradation of the cholesterol side chain in bile acid synthesis. The knockout mice show spontaneous peroxisome proliferation and increased mRNA levels of genes regulated by PPARα. The scp2 null phenotype is similar to that seen in acyl-CoA oxidase (ACO) null mice (Kannenberg et al., J. Biol. Chem. 274(50): 35455-35460, 1999). The scp2 null mice also have affected peroxisomal α-oxidation of phytanic acid (Atshaves et al., 1999). Whether these phenotypes are secondary affects of the gene knockout has yet to be clarified (Seedorf et al., Biochim. Biopsies. Acta. 1486(1): 45-54, 2000).

### EXAMPLE 47

### Tissue Distribution

The human SCP2 gene was cloned from the liver (Yamamoto et al., 1991). The protein has been found most highly expressed in liver, intestine, adrenal and kidney (Baum et al., J. Lipid. Res. 34(5): 729-739, 1993). It is also expressed in lung, brain, testes, ovary and heart, fibroblasts, and placenta (Human SCP-2 (NLTP_HUMAN) record from SWISS—PROT database, ExPasy Website). EST data from normal human tissues in Unigene indicate that the gene is expressed in bone marrow, brain, heart, skeletal muscle, liver, pancreas, prostate, kidney, and lung (SCP2 Genecard record, Genecards Website, Weizmann Institute of Science).

### EXAMPLE 48

### Cellular Localization

The identical C-termini of both SCP-2 and SCP-X contain an SKL peroxisomal targeting signal, however, as much as half of the total SCP-2 is located outside the peroxisome. The SCP-2 N-terminal presequence in the pro-SCP-2 protein strongly modulates intracellular targeting coded for by the C-terminal peroxisomal signal sequence (Gallegos et al., 2001; Stolowich et al., 2002). Other studies indicate that mammalian SCP-2 is found in the cytoplasm or the mitochondria and that SCP-X is found in peroxisomes (Baker et al., DNA Cell Biol. 10(9): 695-698, 1991; Interpro database of proteins [http://www.ebi.ac.uk/interpro/Ientry?ac-IPR003033]).

### EXAMPLE 49

### Role in Disease

SCP-2 levels are altered in diseases where lipid metabolism is abnormal, such as diabetes, Zellweger, Niemann Pick C (NPC) and atherosclerosis (Stolowich et al., 2002). SCP-2 is present in low levels in Zellweger syndrome but is not causal of this syndrome in which the cells are deficient in peroxisomes (SCP2 Genecard recod, Genecards Website, Weizmann Institute of Science). Zellweger patients have no detectable 15 kDa pro-SCP-2 protein or the mature 13 kDa SCP-2 and are deficient in very long chain fatty acid oxidation (Wirtz, Biochem. J. 324(2): 353-360, 1997; van Heusden et al., J. Biol. Chem. 265(7): 4105-4110, 1990).

The SCP-X/SCP-2 gene was investigated as a candidate gene for infantile neuronal ceroid lipofuscinosis. However, despite the gene mapping to the same chromosomal location as markers for this disease, no association could be found between mutations in the SCP-2/SCP-X gene and the disease (Vesa et al., 1994).

NPC1 disease is caused by a mutation in the NPC protein. In this disease cholesterol accumulates in liver lysosomes and the Golgi. This disease shows markedly reduced levels of hepatic 13 kDa SCP-2 as well as accumulation of lipids in lysosomes and Golgi (Roff et al., J. Biol. Chem. 267(22): 15902-15908, 1992).

### EXAMPLE 50

### Relationship to Obesity or Diabetes

Streptozotocin-induced diabetes in rats decreased liver levels of SCP-2 by 60-90% and ovarian levels by 60% (McLean et al., Biol. Reprod. 55(1): 38-46, 1996). Reduced 13 kDa SCP-2 expression in pregnant diabetic mice was associated with pregnancy loss (McLean et al., 1996).

### EXAMPLE 51

### Gene Expression as Measured by SYBR Green Real Time PCR: Red Gastrocnemius Muscle; Exercise Training

AGT-710 gene expression in skeletal muscle of P. obesus increased with exercise training (p=0.016). Gene expression was positively correlated with energy expenditure when all P. obesus were analysed together (R²=0.6508, p=0.001). Gene expression was negatively correlated with blood glucose (R²=0.3903, p=0.016) and activity (R²=0.3543, p=0.030) when all P. obesus were analyzed together. Gene expression was positively correlated with fat oxidation (R²=0.3147, p=0.046) and the change in fat oxidation (R²=0.3452, p=0.035) in exercise trained P. obesus.

### EXAMPLE 52

### Red Gastrocnemius Muscle; Fasting (24 hr)

AGT-710 gene expression was significantly lower in C fed P. obesus when compared to A fed P. obesus (p=0.021). AGT-710 gene expression was negatively correlated with body weight (R²=0.2267, p=0.029) and blood glucose (R²=0.3438, p=0.004) in fed P. obesus. There were no differences in gene expression between the fasted groups, and no correlations with phenotypic variables.

### EXAMPLE 53

### cDNA Microarray Production

RNA extracted from P. obesus was used to generate a cDNA library in the pCMV-SPORT 6 Vector (Invitrogen Life Technologies). Individual cDNA clones were arrayed into 384 well plates (The Australian Genome Research Facitlity, Queensland, Australia). The clones were then PCR amplified using vector complimentary primers (SP6 5′ATT TAG GTG ACA CTA TAG 3′ [SEQ ID NO:10]; T7: 5′-TAATACGACT CACTATAGGG-3′ [SEQ ID NO:11). PCR amplification of each clone was performed in a GeneAmp PCR System 9700 thermal cycler (PE Applied BioSystems, Sunnyvale, Calif.) for 35 cycles of denaturation at 95° C. for 30 sec, annealing at 56° C. for 30 sec and extension at 72° C. for 120 sec. A final extension step was perfonned at 72° C. for 5 min. Products were visualized by TAE agarose gel (1.5% w/v) electrophoresis at 6 V/cm for 90 min to ensure successful amplification had taken place.

PCR products were purified using the ArrayIt vacuum manifold system (TeleChem International, Sunnyvale, Calif.) and resuspended in 20 μL of 1× spotting solution (TeleChem) at a concentration of 0.5 mg/ml in 384 well plate format. 5 μL of the resuspended purified cDNA solution was transferred to 384 well uniplates (Whatman Inc, Clifton, USA). This cDNA was arrayed onto Super Amine Microarray Substrates (TeleChem) using a Chip Writer Pro robotic arrayer (Virtek, Toronto, Canada) fitted with 16 Stealth SMP-03 quill tipped microarray pins (Telechem). The distance between adjacent cDNA spots was 200 μM. Each pin drew 0.25 μL of cDNA and deposited approximately 0.6 nL on each slide. Humidity was maintained between 55-65% during printing. Approximately 12,000 elements were printed per microarray. Spotted DNAs were allowed to dry overnight, after which the slides were washed and blocked as recommended by the manufacturer (TeleChem).

### EXAMPLE 54

### RNA Extraction

Total RNA was extracted from tissue in a two-step process utilising Trizol (Invitrogen Life Technologies, Carlsbad, USA) and RNeasy (Qiagen, Hilden, Germany) protocols. The tissue samples were lysed in 1.5 ml of Trizol (Invitrogen Life Technologies) and homogenised using a Ystral Homogeniser (model D-7801, Dottingen, Germany). 300 μL of chloroform was added to the homogenate, which was then mixed, transferred into a fresh 2 ml tube and incubated at room temperature for 3 min. The homogenates were then separated by centrifugation at 13 000×g for 15 min (4° C.). Following centrifugation the aqueous supernatant was collected in 2 ml tubes and an equal volume of 70% v/v ethanol added, with the solution mixed by pipetting. 700 μL of the sample was then transferred via pipette into RNeasy kit mini spin columns (Qiagen) placed in 2 ml tubes (supplied) for purification. Initially the sample was centrifuged at 10 000×g for 20 sec. The flow-through was then poured back into the column and the centrifngation repeated. Further purification was performed according to manufacturer's instructions and the RNA was eluted using RNAase free water.

Following purification, total RNA integrity, quantity and concentration was assessed using the RNA 6000 Nano Assay (Agilent Technologies, Palo Alto, USA) with the Agilent 2100 Bioanalyser (Agilent Technologies) as per the manufacturer's instructions. This system utilises capillary electrophoresis to separate and detect nucleic acid fragments by size through the interconnected micro channels on a Nano chip (Agilent Technologies). Good quality RNA is signified by an electropherogram displaying a marker peak, and two ribosomal peaks of which the 18s band is at an approximate ratio of 1:2 to the 28s band.

### EXAMPLE 55

### Indirect Labeling of cDNA

Fluorescently labelled cDNA was prepared from 20 μg of total RNA using an indirect labelling method. cDNA synthesis was performed in a 30 μL reaction containing 5 μg oligo-dT primer, 400U SuperscriptII (Invitrogen), 1x first strand buffer, 0.01 M DTT, 0.5 mM of each dATP, dCTP and dGTP, 0.150 mM dTTP (Amersham, Buckinghamshire, UK) and 0.2 mM aminoallyl-dUTP (Sigma, St. Louis, Mo.). Synthesis was conducted in a GeneAmp PCR System 9700 (PE Applied Systems) at 42° C. for 2 hours. The reaction was stopped by addition of 5 μL of 0.5 M EDTA and RNA was hydrolyzed by addition of 20 μl of 1 M NaOH at 70° C. for 20 minutes. The reaction was neutralized with 25 μL of 1 M HEPES and the cDNA was purified using QIAGEN PCR purification kits according to manufacturer's instructions and eluted in nuclease-free water. The cDNA was concentrated using Microcon30 spin columns (Millipore, Bedford, Mass.) and the volume retrieved dried down under vacuum. The cDNA pellet was resuspended in 0.09 M sodium bicarbonate and coupled to Cy3 or Cy5 monofunctional NHS ester reactive dye (Amersham). The coupling reaction was conducted in the dark for 1 hour.

Dye-coupled cDNA was purified using Qiagen PCR purification columns, combined and added to 10 μg of Human Cot1 DNA (Invitrogen Life Technologies). The cDNAs were again concentrated with Microcon 30 spin columns (Millipore). The cDNA was hybridized in a 40 μL volume containing the labeled cDNA, 20×SSC, 8 μg PolydA, 2.5× Denhardt's solution, 4 μg yeast tRNA and 10% w/v SDS. The cDNA was then denatured at 98° C. for 2 min and maintained at 60° C. until required. 38 μL of the hybridization solution was applied to a cover slip and then mounted onto an array slide. Hybridization was conducted in a humid hybridisation chamber, in a hybridization oven, at 60° C. for 16 hours. Following hybridization the array slides were removed from their chamber and washed for 2 min in each of a 0.5×SSC and 0.1% w/v SDS, 0.5×SSC and 0.01% w/v SDS, 0.6×SSC and 0.06% w/v SDS solution. The array slides were dried in a centrifuge for 1 min at 500×g.

### EXAMPLE 56

### Image Acquisition and Data Analysis

Fluorescent images of the microarrays were acquired using a ScanArray Lite confocal laser scanner (Perkin Elmer) or GenePix 4000B scanner (Axon Instruments) and the images were analysed using GenePix Pro 4.0 and Acuity 2.0 (Axon Instruments) and GeneSight 3.0 (Biopiscovery, Sunnyvale, Calif.). Slides were scanned for both Cy3 and Cy5 signal at a 10 μM pixel resolution. Laser intensity and amplification of the photomultiplier tubes were adjusted to ensure approximately equal overall signal intensity for both Cy3 and Cy5. Data obtained from the scanner was imported into Gene Pix Pro (Version 4.0, Axon Instruments). False colour images were generated for each dye and combined to provide a representation of the relative Cy3 and Cy5 intensities. Individual cDNA spots were flagged if spot size was too small, if the overall signal intensity was too low, or if the Cy3 and Cy5 signal intensities within the spots were not linearly related. GenePix allows for the “flagging” of bad elements (defined by present GenePix parameters as feature signal intensity; feature background; element morphology; elements size and the percentage of pixels greater than feature background) that were then excluded from further analysis.

Median Cy3 and Cy5 signal intensities for each cDNA spot were imported from Genepix and data transformation conducted using Genesight (Version 3.0, BioDiscovery Inc, Los Angeles, USA). Signal intensities were corrected for local background and low expression values were omitted. The ratio of Cy3 to Cy5 was calculated and the data logarithmically transformed (base2). Signal intensity was normalized to the mean intensity of all respective signal intensities, providing a relative measure of gene expression for each element on the microarray slide. Gene expression analysis between control animals and animals separated for 4 days was assessed using an independent samples t-test. Differential gene expression as measured by microarray was screened for significance at p<0.05.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul et al., Nucl. Acids Res. 25: 3389, 1997.

Antonenkov et al., Isolation and subunit composition of native sterol carrier protein 2/3-oxoacyl-coenzyme A thiolase from normal rat liver peroxisomes. Protein Expr Purif. 18(3): 249-256, 2000.

Antonenkov et al., Substrate specificities of 3-oxoacyl-CoA thiolase A and sterol carrier protein 2/3-oxoacyl-CoA thiolase purified from normal rat liver peroxisomes. Sterol carrier protein 2/3-oxoacyl-CoA thiolase is involved in the metabolism of 2-methyl-branched fatty acids and bile acid intermediates. J. Biol. Chem. 272(41): 26023-26031, 1997.

Atshaves et al., Expression and intracellular processing of the 58 kDa sterol carrier protein-2/3-oxoacyl-CoA thiolase in transfected mouse L-cell fibroblasts. J Lipid Res. 40(4): 610-622, 1999.

Australian Institute of Health and Welfare (AIWH), Australia's Health, 2002, Canberra: AIWH.

Ausubel et al. “Current Protocols in Molecular Biology” John Wiley & Sons Inc, Unit 19.3, Chapter 15, 1994-1998.

Baker et al., Similarity between the amino-terminal portion of mammalian 58-kD sterol carrier protein (SCPx) and Escherichia coli acetyl-CoA acyltransferase: evidence for a gene fusion in SCPx. DNA Cell Biol 10(9): 695-698, 1991.

Barnes et al., The Caenorhabditis elegans behavioral gene unc-24 encodes a novel bipartite protein similar to both erythrocyte band 7.2 (stomatin) and nonspecific lipid transfer protein. J. Neurochem. 67(1): 46-57, 1996.

Barnett et al., Diabete Nutr. Metab. 8: 42-47, 1995.

Barnett et al., A cross-sectional and short-term longitudinal characterisation of NIDDM in Psammomys obesus. Diabetologia 377: 671-676, 1994a.

Barnett et al., The effect of restricting energy intake on diabetes in Psammomys obesus. Int. J. Obesity 18: 789-794, 1994b.

Baum et al., Regulation of sterol carrier protein-2 gene expression in rat liver and small intestine. J. Lipid Res. 34(5): 729-739, 1993.

Bhattacharjee et al., Genomic organization and chromosomal localization of the Asna1 gene, a mouse homologue of a bacterial arsenic-translocating ATPase gene. Gene 272: 291-299, 2001.

Bonner and Laskey, Eur. J. Biochem. 46: 83, 1974.

Bouchard, The genetics of Obesity, Boca Raton: CRC Press, 1994.

Boulkroun et al., Characterization of rat NDRG2 (N-myc downstream-regulated gene 2), a novel early mineralqcorticoid-specific induced gene. J. Biol. Chem. 277(35): 31506-31515, 2002.

Bun-ya et al., Thiolase involved in bile acid formation. J Biochem (Tokyo) 123(2): 347-352, 1998.

Collier et al., Development of obesity and insulin resistance in the Israeli sand rat (Psammomys obesus). Does leptin play a role? Ann. New York Acad. Sci. 827. 50-63, 1997a.

Collier et al., Exp. Clin. Endocrinol. Diabetes 105: 36-37, 1997b. DBH4 record from Pfam database of protein families Website, Sanger Centre http://www.sanger.ac.uk/cgi-bin/Pfam/swisspfamget.pl?name-DHB4 HUMAN

Dunstan et al., The Australian Diabetes, Obesity and Lifestyle Study (AusDiab)—methods and response rates. Diabetes Res. Clin. Pract. 57: 119-129, 2002.

Ferdinandusse et al., Peroxisomal fatty acid oxidation disorders and 58 kDa sterol carrier protein X (SCPx). Activity measurements in liver and fibroblasts using a newly developed method. J Lipid Res. 41(3): 336-342, 2000.

Festa et al., Elevated levels of acute-phase proteins and plasminogen activator inhibitor-1 predict the development of type 2 diabetes: the insulin resistance atherosclerosis study. 51(4): 1131-1137,2002.

Fuchs et al., Disruption of the sterol carrier protein 2 gene in mice impairs biliary lipid and hepatic cholesterol metabolism. J Biol Chem. 276(51): 48058-48065, 2001.

Fuchs et al., Sterol carrier protein 2 participates in hypersecretion of biliary cholesterol during gallstone formation in genetically gallstone-susceptible mice. Biochem J. 336(1): 33-37, 1998.

Hoffstedt et al., The common −675 4G/5G polymorphism in the plasminogen activator inhibitor-1 gene is strongly associated with obesity. Diabetologia 45(4): 584-587, 2002.

Human SCP-2 (NLTP_HUMAN) record from SWISS-PROT database, ExPasy Website http://kr.exipasy.org/cgi-bin/niceprot.pl?P22307

Interpro database of proteins http://www.ebi.ac.uk/interpro/IEntry?ac=IPR003033

Ito et al., Hepatic cholesterol metabolism in patients with cholesterol gallstones: enhanced intracellular transport of cholesterol. Gastroenterology 110(5): 1619-1627, 1996.

Jiang et al., Ahi-1, a novel gene encoding a modular protein with WD40-repeat and SH3 domains, is targeted by the Ahi-1 and Mis-2 provirus integrations. J. Virol. 76(18): 9046-9059, 2002.

Kalaydjieva et al., N-myc downstream-regulated gene 1 is mutated in hereditary motor and sensory neuropathy-Lom. Am. J. Hum. Genet. 67(1): 47-58, 2000.

Kannenberg et al., Aberrant oxidation of the cholesterol side chain in bile acid synthesis of sterol carrier protein-2/sterol carrier protein-x knockout mice. J Biol Chem. 274(50): 35455-35460, 1999.

Kawata et al., Modulation of cholesterol 7 alpha-hydroxylase activity by nonspecific lipid transfer protein in human liver--possibly altered regulation of its cytosolic level in patients with gallstones. Clin Chim Acta.197(3): 201-208, 1991.

Kopelman et al., ASO consensus statement on obesity. UK Association for the Study of Obesity, Int. J. Obesity 18: 188-191, 1994.

Kopelman, Obesity as a medical problem. Nature 404: 635-643, 2000.

Kurdi-Haidar et al., Isolation of the ATP-binding human homolog of the arsA component of the bacterial arsenite transporter. Genomics 36: 486-491, 1996.

Kurdi-Haidar et al., Biochemical characterization of the human arsenite-stimulated ATPase (hASNA-I). J. Biol. Chem. 273: 22173-22176, 1998a.

Kurdi-Haidar et al., Dual cytoplasmic and nuclear distribution of the novel arsenite-stimulated human ATPase (hASNA-I). J. Cell Biochem. 71: 1-10, 1998b.

Kurdi-Haidar et al., Chromosomal localization and genomic structure of the human arsenite-stimulated ATPase (hASNA-1). Somat. Cell Molec. Genet. 24: 307-311, 1998c.

Kurdi-Haidar et al., Immunohistochemical analysis of the distribution of the human ATPase (bASNA-1) in normal tissues and its overexpression in breast adenomas and carcinomas. J. Histochem. Cytochem. 46:1243-1248, 1998d.

Marmur and Doty, J. Mol. Biol. 5: 109, 1962.

Marx, Unraveling the causes of diabetes. Science 296: 686-689, 2002.

Mavri et al., Subcutaneous abdominal, but not femoral fat expression of plasminogen activator inhibitor-1 (PAI-1) is related to plasma PAI-1 levels and insulin resistance and decreases after weight loss. Diabetologia 44(11): 2025-2031, 2001.

McLean et al., Altered ovarian sterol carrier protein expression in the pregnant streptozotocin-treated diabetic rat. Biol Reprod. 55(1): 38-46, 1996.

Mertens and Van Gaal, Obesity, haemostasis and the fibrinqlytic system. Obes. Rev. 3(2): 85-101, 2002.

Mokdad et al., The spread of the obesity epidemic in the United States, 1991-1998. JAMA 282(16): 1519-1522, 1999.

Murphy and Schroeder, Sterol carrier protein-2 mediated cholesterol esterification in transfected L-cell fibroblasts. Biochim Biophys Acta. 1345(3): 283-292, 1997.

Must et al., The disease burden associated with overweight and obesity. JAMA 282(16): 1523-1529, 1999.

National Health and Medical Research Council, Acting on Australia's weight: A strategy for the prevention of overweight and obesity. Canberra: National Health and Medical Research Council, 1996.

Ohba et al., The structure of the human sterol carrier protein X/sterol carrier protein 2 gene (SCP2). Genomics 24: 370-374, 1994.

Okuda and Kondoh, Identification of new genes ndr2 and ndr3 which are related to Ndr1/RTP/Drg1 but show distinct tissue specificity and response to N-myc. Biochem. Biophys. Res. Commun. 266(1): 208-215, 1999.

Olsen and Uhler, Inhibition of protein kinase-A by overexpression of the cloned human protein kinase inhibitor. Molec. Endocr. 5: 1246-1256, 1991.

Ossendorp et al., Tissue-specific distribution of a peroxisomal 46-kDa protein related to the 58-kDa protein (sterol carrier protein x; sterol carrier protein 2/3-oxoacyl-CoA thiolase). Arch Biochem Biophys. 334(2): 251-260, 1996.

Pandolfi et al., Plasminogen activator inhibitor type 1 is increased in the arterial wall of type II diabetic subjects. Arterioscler Thomb. Vasc. Biol 21(8): 1378-1382, 2001.

Puglielli et al., Modulation of intrahepatic cholesterol trafficking: evidence by in vivo antisense treatment for the involvement of sterol carrier protein-2 in newly synthesized cholesterol transport into rat bile. Biochem J. 317(3): 681-687, 1996.

Qu et al., Characterization and expression of three novel differentiation-related genes belong to the human NDRG gene family. Mol. Cell. Biochem. 229(1-2): 35-44, 2002.

Ravussin, Metabolic differences and the development of obesity. Metabolism 44(3): 12-14, 1995.

Roff et al., Deficiencies in sex-regulated expression and levels of two hepatic sterol carrier proteins in a murine model of Niemann-Pick type C disease. J Biol Chem. 267(22): 15902-15908, 1992.

Samad et al., Insulin continues to induce plasminogen activator inhibitor 1 gene expression in insulin-resistant mice and adipocytes. Mol. Med. 6(8): 680-692, 2000.

Schroeder et al., Probing the ligand binding sites of fatty acid and sterol carrier proteins: effects of ethanol. Biochemistry. 34(37): 11919-11927, 1995.

SCP2 Genecard record, Genecards Website, Weizmann Institute of Science http://bioinfo.weizmann.ac.il/cards-bin/carddisp?SCP2&search=scp2&suff=txt

SCP-2 record from Pfam database of protein families Website, Sanger Centre http://www.sanger.ac.uk/cgi-bin/Pfam/getacc?PF02036

Seedorf et al., Defective peroxisomal catabolism of branched fatty acyl coenzyme A in mice lacking the sterol carrier protein-2/sterol carrier protein-x gene function. Genes Dev. 12(8): 1189-1201, 1998.

Seedorf et al., Sterol carrier protein-2. Biochim Biophys Acta. 1486(1): 45-54, 2000. Review.

Shafrir and Gutman, Psammomys obesus of the Jerusalem colony: a model for nutritionally induced, non-insulin-dependent diabetes. J. Basic Clin. Physiol. Pharm. 4: 83-99, 1993.

Starodub et al., Sterol carrier protein-2 localization in endoplasmic reticulum and role in phospholipid formation. Am J Physiol Cell Physiol. 279(4): C1259-1269, 2000.

Sterol Carrier Protein 2 record, OMIM Website http://www.ncbi.nlm.nih.gov/htbin-post/Omim/dispmim?184755

Szyperski et al., NMR determination of the secondary structure and the three-dimensional polypeptide backbone fold of the human sterol carrier protein 2. FEBS Lett. 335(1): 18-26, 1993.

Vaisanen et al. , Vaisanen S B, Humphries S E, Luong L A. Penttila I. Bouchard C. Rauramaa R. Regular exercise, plasminogen activator inhibitor-1 (PAI-1) activity and the 4G/5G promoter polymorphism in the PAI-1 gene. Thomb. Haemost. 82(3): 1117-1120, 1999.

van Heusden et al., Chinese hamster ovary cells deficient in peroxisomes lack the nonspecific lipid transfer protein (sterol carrier protein 2). J Biol Chem. 265(7): 4105-4510, 1990.

Vesa et al., Assignment of sterol carrier protein X/sterol carrier protein 2 to 1p32 and its exclusion as the causative gene for infantile neuronal ceroid lipofuscinosis. Hum. Molec. Genet. 3: 341-346, 1994.

Walder et al., The effect of dietary energy restriction on body weight gain and the development of noninsulin-dependent diabetes mellitus (NIDDM) in Psammomys obesus. Obesity Res. 5:-193-200, 1997a.

Wanders et al., Identification of the newly discovered 58 kDa peroxisomal thiolase SCPx as the main thiolase involved in both pristanic acid and trihydroxycholestanoic acid oxidation: implications for peroxisomal beta-oxidation disorders. J Inherit Metab Dis. 21(3): 302-305, 1998.

Wanders et al., Sterol carrier protein X (SCPx) is a peroxisomal branched-chain beta-ketothiolase specifically reacting with 3-oxo-pristanoyl-CoA: a new, unique role for SCPx in branched-chain fatty acid metabolism in peroxisqmes. Biochem Biophys Res Commun. 236(3): 565-569,1997.

Welch et al., Assignment of the mouse sterol carrier protein gene (Scp2) to chromosome 4. Mammalian Gengme 7: 624-625, 1996.

Wirtz, Phospholipid transfer proteins revisited. Biochem J. 324(2): 353-360, 1997. Review.

World Trade Organisation. Obesity. Preventing and managing the global epidemic. Report of a WHO Consultation on Obesity. Geneva: World Health Organisation, 1998.

Yamamoto et al., Cloning and expression of a cDNA encoding human sterol carrier protein 2. Proc Natl Acad Sci USA. 88(2): 463-467, 1991.

Yamamoto, Localization of human sterol carrier protein 2 gene and cDNA expression in COS-7 cells. Hokkaido Igaku Zasshi 67: 839-848, 1992.

Zanlungo et al., Sterol carrier protein 2 gene transfer changes lipid metabolism and enterohepatic sterol circulation in mice. Gastroenterology. 119(6): 1708-1719, 2000.

Zhou et al., Characterization of the human NDRG gene family: a newly identified member, NDRG4, is specifically expressed in brain and heart. Genomics 73(1): 86-97, 2001.

Zimmet, Kelly West Lecture 1991. Challenges in diabetes epidemiology—from West to the rest. Diabetes Care 15: 232-252, 1992.

1. An isolated nucleic acid molecule comprising a sequence of nucleotides encoding or complementary to a sequence encoding a molecule or derivative or homolog thereof wherein said nucleic acid molecule is expressed in a larger amount in hypothalamus tissue of obese animals compared to lean animals or in fasted animals compared to fed animals wherein the nucleic acid molecule is selected from:
(i) a nucleic acid molecule comprises a nucleotide sequence as set forth in SEQ ID NO:1 or a nucleotide sequence having at least 40% identity thereto or a nucleotide sequence capable of hybridizing to SEQ ID NO:1 or its complementary form under low stringency conditions;
(ii) a nucleic acid molecule comprises a nucleotide as set forth in SEQ ID NO:2 or a nucleotide sequence having at least 40% identity thereto or a nucleotide sequence capable of hybridizing to SEQ ID NO:2 or its complementary form under low stringency conditions;
(iii) a nucleic acid molecule comprises a nucleotide sequence as set forth in SEQ ID NO:3 or a nucleotide sequence having at least 40% identity thereto or a nucleotide sequence capable of hybridizing to SEQ ID NO:3 or its complementary form under low stringency conditions;
(iv) a nucleic acid molecule comprises a nucleotide sequence as set forth in SEQ ID NO:4 or a nucleotide sequence having at least 40% identity thereto or a nucleotide sequence capable of hybridizing to SEQ ID NO:4 or its complementary form under low stringency conditions;
(v) a nucleic acid molecule comprises a nucleotide sequence as set forth in SEQ ID NO:5 or a nucleotide sequence having at least 40% identity thereto or a nucleotide sequence capable of hybridizing to SEQ ID NO:5 or its complementary form under low stringency conditions;
(vi) a nucleic acid molecule comprises a nucleotide sequence as set forth in SEQ ID NO:6 or a nucleotide sequence having at least 40% identity thereto or a nucleotide sequence capable of hybridizing to SEQ ID NO:6 or its complementary form under low stringency conditions;
(vii) a nucleic acid molecule comprises a nucleotide sequence as set forth in SEQ ID NO:7 or a nucleotide sequence having at least 40% identity thereto or a nucleotide sequence capable of hybridizing to SEQ ID NO:7 or its complementary form under low stringency conditions;
(viii) a nucleic acid molecule comprises a nucleotide sequence as set forth in SEQ ID NO:8 or a nucleotide sequence having at least 40% identity thereto or a nucleotide sequence capable of hybridizing to SEQ ID NO:8 or its complementary form under low stringency conditions; and
(ix) a nucleic acid molecule comprises a nucleotide sequence as set forth in SEQ ID NO:9 or a nucleotide sequence having at least 40% identity thereto or a nucleotide sequence capable of hybridizing to SEQ ID NO:9 or its complementary form under low stringency conditions.

2. The isolated nucleic acid molecule of claim 1 wherein the nucleic acid molecule comprises the nucleotide sequence as set forth in SEQ ID NO:1.

3. The isolated nucleic acid molecule of claim 1 wherein the nucleic acid molecule comprises the nucleotide sequence as set forth in SEQ ID NO:2.

4. The isolated nucleic acid molecule of claim 1 wherein the nucleic acid molecule comprises the nucleotide sequence as set forth in SEQ ID NO:3.

5. The isolated nucleic acid molecule of claim 1 wherein the nucleic acid molecule comprises the nucleotide sequence as set forth in SEQ ID NO:

6. The isolated nucleic acid molecule of claim 1 wherein the nucleic acid molecule comprises the nucleotide sequence as set forth in SEQ ID NO:5.

7. The isolated nucleic acid molecule of claim 1 wherein the nucleic acid molecule comprises the nucleotide sequence as set forth in SEQ ID NO:6.

8. The isolated nucleic acid molecule of claim 1 wherein the nucleic acid molecule comprises the nucleotide sequence as set forth in SEQ ID NO:7.

9. The isolated nucleic acid molecule of claim 1 wherein the nucleic acid molecule comprises the nucleotide sequence as set forth in SEQ ID NO:8.

10. The isolated nucleic acid molecule of claim 1 wherein the nucleic acid molecule comprises the nucleotide sequence as set forth in SEQ ID NO:9.

11. An isolated molecule comprising a sequence of nucleotides or amino acids encoded by a nucleic acid molecule which is expressed in a larger amount in hypothalamus tissue of obese animals compared to lean animals or in fasted animals compared to fed animals wherein the isolated molecule is encoded by a nucleic acid molecule selected from:
(i) a nucleic acid molecule as set forth in SEQ ID NO:1 or a nucleotide sequence having at least about 40% identity to SEQ ID NO:1 or a nucleotide sequence capable of hybridizing to SEQ ID NO:1 or its complementary form under low stringency conditions;
(ii) a nucleic acid molecule as set forth in SEQ ID NO:2 or a nucleotide sequence having at least about 40% identity to SEQ ID NO:2 or a nucleotide sequence capable of hybridizing to SEQ ID NO:2 or its complementary form under low stringency conditions;
(iii) a nucleic acid molecule as set forth in SEQ ID NO:3 or a nucleotide sequence having at least about 40% identity to SEQ ID NO:3 or a nucleotide sequence capable of hybridizing to SEQ ID NO:3 or its complementary form under low stringency conditions;
(iv) a nucleic acid molecule as set forth in SEQ ID NO:4 or a nucleotide sequence having at least about 40% identity to SEQ ID NO:4 or a nucleotide sequence capable of hybridizing to SEQ ID NO:4 or its complementary form under low stringency conditions;
(v) a nucleic acid molecule as set forth in SEQ ID NO:5 or a nucleotide sequence having at least about 40% identity to SEQ ID NO:5 or a nucleotide sequence capable of hybridizing to SEQ ID NO:5 or its complementary form under low stringency conditions;
(vi) a nucleic acid molecule as set forth in SEQ ID NO:6 or a nucleotide sequence having at least about 40% identity to SEQ ID NO:6 or a nucleotide sequence capable of hybridizing to SEQ ID NO:6 or its complementary form under low stringency conditions;
(vii) a nucleic acid molecule as set forth in SEQ ID NO:7 or nucleotide sequence having at least about 40% identity to SEQ ID NO:7or a nucleotide sequence capable of hybridizing to SEQ ID NO:7 or its complementary form under low stringency conditions;
(viii) nucleic acid molecule as set forth in SEQ ID NO:8 or a nucleotide sequence having at least about 40% identity to SEQ ID NO:8 or a nucleotide sequence capable of hybridizing to SEQ ID NO:8 or its complementary form under low stringency conditions; and
(ix) a nucleic acid molecule set forth in SEQ ID NO:9 or a nucleotide sequence having at least about 40% identity to SEQ ID NO:9 or a nucleotide sequence capable of hybridizing to SEQ ID NO:9 or its complementary form under low stringency conditions.

12. The isolated molecule of claim 11 wherein the molecule is a protein.

13. The isolated protein of claim 12 encoded by a nucleotide sequence set forth in SEQ ID NO:1.

14. The isolated protein of claim 12 encoded by a nucleotide sequence set forth in SEQ ID NO:2.

15. The isolated protein of claim 12 encoded by a nucleotide sequence as set forth in SEQ ID NO:3.

16. The isolated protein of claim 12 encoded by a nucleotide sequence as set forth in SEQ ID NO:4.

17. The isolated protein of claim 12 encoded by a nucleotide sequence as set forth in SEQ ID NO:5.

18. The isolated protein of claim 12 encoded by a nucleotide sequence as set forth in SEQ ID NO:6.

19. The isolated protein of claim 12 encoded by a nucleotide sequence as set forth in SEQ ID NO:7

20. The isolated protein of claim 12 encoded by a nucleotide sequence as set forth in SEQ ID NO:8.

21. The isolated protein of claim 12 encoded by a nucleotide sequence as set forth in SEQ ID NO:9.

22. An isolated protein selected from the list consisting of:
(i) a protein encoded by a nucleotide sequence substantially as set forth in SEQ ID NO:1 or a derivative, homolog or analog thereof or a sequence encoding an amino acid sequence having at least about 40% similarity to this sequence or a derivative, homolog, analog, chemical equivalent or mimetic of said protein;
(ii) a protein encoded by a nucleotide sequence substantially as set forth in SEQ ID NO:2 or a derivative, homolog or analog thereof or a sequence encoding an amino acid sequence having at least about 40% similarity to this sequence or a derivative, homolog, analog, chemical equivalent or mimetic of said protein;
(iii) a protein encoded by a nucleotide sequence substantially as set fort in SEQ ID NO:3 or a derivative, homolog or analog thereof or a sequence encoding an amino acid sequence having at least about 40% similarity to this sequence or a derivative, homolog, analog, chemical equivalent or mimetic of said protein;
(iv) a protein encoded by a nucleotide sequence substantially as set forth in SEQ ID NO:4 or a derivative, homolog or analog thereof or a sequence encoding an amino acid sequence having at least about 40% similarity to this sequence or a derivative, homolog, analog, chemical equivalent or mimetic of said protein;
(v) a protein encoded by a nucleotide sequence substantially as set forth in SEQ ID NO:5 or a derivative, homolog or analog thereof or a sequence encoding an amino acid sequence having at least about 40% similarity to this sequence or a derivative, homolog, analog, chemical equivalent or mimetic of said protein;
(vi) a protein encoded by a nucleotide sequence substantially as set forth in SEQ ID NO:6 or a derivative, homolog or analog thereof or a sequence encoding an amino acid sequence having at least about 40% similarity to this sequence or a derivative, homolog, analog, chemical equivalent or mimetic of said protein;
(vii) a protein encoded by a nucleotide sequence substantially as set forth in SEQ ID NO:7 or a derivative, homolog or analog thereof or a sequence encoding an amino acid sequence having at least about 40% similarity to this sequence or a derivative, homolog, analog, chemical equivalent or mimetic of said protein;
(viii) a protein encoded by a nucleotide sequence substantially as set forth in SEQ ID NO:8 or a derivative, homolog or analog thereof or a sequence encoding an amino acid sequence having at least about 40% similarity to this sequence or a derivative, homolog, analog, chemical equivalent or mimetic of said protein;
(ix) a protein encoded by a nucleotide sequence substantially as set forth in SEQ ID NO:9 or a derivative, homolog or analog thereof or a sequence encoding an amino acid sequence having at least about 40% similarity to this sequence or a derivative, homolog, analog, chemical equivalent or mimetic of said protein;
(x) a protein encoded by a nucleotide acid molecule capable of hybridizing to the nucleotide sequence as set forth in SEQ ID NO:1 or a derivative, homolog or analog thereof under low stringency conditions;
(xi) a protein encoded by a nucleic acid molecule capable of hybridizing to the nucleotide sequence as set forth in SEQ ID NO:2 or a derivative, homolog or analog thereof under low stringency conditions;
(xii) a protein encoded by a nucleic acid molecule capable of hybridizing to the nucleotide sequence as set forth in SEQ ID NO:3 or a derivative, homolog or analog thereof under low stringency conditions;
(xiii) a protein encoded by a nucleic acid molecule capable of hybridizing to the nucleotide sequence as set forth in SEQ ID NO:4 or a derivative, homolog or analog thereof under low stringency conditions;
(xiv) a protein encoded by a nucleic acid molecule capable of hybridizing to the nucleotide sequence as set forth in SEQ ID NO:5 or a derivative, homolog or analog thereof under low stringency conditions;
(xv) a protein encoded by a nucleic acid molecule capable of hybridizing to the nucleotide sequence as set forth in SEQ ID NO:6 or a derivative, homolog or analog thereof under low stringency conditions;
(xvi) a protein encoded by a nucleic acid molecule capable of hybridizing to the nucleotide sequence as set forth in SEQ ID NO:7 or a derivative, homolog or analog thereof under low stringency conditions;
(xvii) a protein encoded by a nucleic acid molecule capable of hybridizing to the nucleotide sequence as set forth in SEQ ID NO:8 or a derivative, homolog or analog thereof under low stringency conditions; and
(xviii) a protein encoded by a nucleic acid molecule capable of hybridizing to the nucleotide sequence as set forth in SEQ ID NO:9 or a derivative, homolog or analog thereof under low stringency conditions.

23. A method for modulating expression of one of AGT-701, AGT-702, AGT-704, AGT-705, AGT-706, AGT-707; AGT-708, AGT-709 and/or AGT-710 in a mammal, said method comprising contacting of AGT-701, AGT-702, AGT-704, AGT-705, AGT-706, AGT-707, AGT-708, AGT-709 and/or AGT-710 with an effective amount of a modulator of AGT-701, AGT-702, AGT-704, AGT-705, AGT-706, AGT-707, AGT-708, AGT-709 and/or AGT-710 expression for a time and under conditions sufficient to up-regulate or down-regulate or otherwise modulate expression of AGT-701, AGT-702, AGT-704, AGT-705, AGT-706, AGT-707, AGT-708, AGT-709 and/or AGT-710.

24. A method of modulating activity of AGT-701, AGT-702, AGT-704, AGT-705, AGT-706, AGT-707, AGT-708, AGT-709 and/or AGT-710 in a mammal, said method comprising administering to said mammal a modulating effective amount of a molecule for a time and under conditions sufficient to increase or decrease of AGT-701, AGT-702, AGT-704, AGT-705, AGT-706, AGT-707, AGT-708, AGT-709 and/or AGT-710 activity.

25. A method of treating a mammal suffering from a condition characterized by one or more symptoms or imter alia a myopathy, obesity, anorexia, weight maintenance, diabetes, disorders associated with mitochrondrial dysfunction, genetic disorders, cancer impaired muscle development, heart diseas, inflammation, disorders associated with the immune system, infertility, disease associated with the brain, and metabolic energy levels, said method comprising administering to said mammal an effective amount of an agent for a time and under conditions sufficient to modulate the expression of AGT-701, AGT-702, AGT-704, AGT-705, AGT-706, AGT-707, AGT-708, AGT-709 and/or AGT-710.

26. A method of treating mammal suffering from a disease condition characterized by one or more symptoms of inter alia a myopathy, obesity, anorexia, weight maintenance, diabetes, inflammation, disorders associated with the immune system, infertility, disease associated with the brain, and/or metabolic energy levels, said method comprising administering to said mammal an effective amount of AGT-701, AGT-702, AGT-704, AGT-705, AGT-706, AGT-707, AGT-708, AGT-709 and/or AGT-710.

27-29. (canceled)

30. A composition comprising a modulator of AGT-701, AGT-702, AGT-704, AGT-705, AGT-706, AGT-707, AGT-708, AGT-709 and/or AGT-710 expression AGT-701, AGT-702, AGT-704, AGT-705, AGT-706, AGT-707, AGT-708, AGT-709 and/or AGT-710 activity and one or more pharmaceutically acceptable carriers and/or diluents.

31. A method for detecting AGT-701, AGT-702, AGT-704, AGT-705, AGT-706, AGT-707 AGT-708, AGT-709 and/or AGT-710 or a derivative or homolog thereof in a biological sample from a subject, said method comprising contacting said biological sample with an antibody specific for AGT-701, AGT-702, AGT-704, AGT-705, AGT-706, AGT-707, AGT-708, AGT-709, AGT-710 or their antigenic derivatives or homologs for a time and under conditions sufficient for a complex to form, and then detecting said complex.
